(19) United States
(12) Patent Application Publication
(10) Pub. No.: US 2026/0327661 A1
(43) Pub. Date: Oct. 22, 2026

(54) GENETIC SWITCH FOR TIME-CONTROLLED ACTIVATION OF FERMENTATION PROTEINS

(71) Applicant: Gevo, Inc., Englewood, CO (US)

(72) Inventors: Matthew Marzo, Denver, CO (US); Chris Smith, Bozeman, MT (US); Kent Evans, Littleton, CO (US)

(21) Appl. No.: 19/540,937

(22) Filed: Feb. 16, 2026

Related U.S. Application Data
(60) Provisional application No. 63/760,554, filed on Feb. 19, 2025.

(51) Int. Cl. C12P 7/16; C12N 9/04; C12N 9/10; C12N 9/88; C12N 15/52

(52) U.S. Cl. CPC C12P 7/16; C12N 9/0006; C12N 9/1022; C12N 9/88; C12N 15/52; C12Y 101/01086; C12Y 202/01006; C12Y 402/01009

(57) ABSTRACT

Disclosed are systems and methods for producing renewable alcohols by fermentative processes using selected promoters that differentially regulate the expression of genes encoding fermentation enzymes during the propagation and production phases of the fermentative processes. These systems and methods are able to increase production efficiency and yield in the conversion of sugars to renewable alcohols by fermentation in microbes. This is accomplished by the use of novel nucleic acid sequences that act as genetic switches to differentially control gene expression based on extra cellular stress; namely, stresses relating to fermentation processes. These sequences present a novel method for controlling endogenous gene expression without the need for the addition of heterlogous chemicals or signaling molecules.

### RELATED APPLICATIONS

This application claims the benefit of U.S. Provisional Application No. 63/760,554, entitled “Genetic Switch for Time-Controlled Activation of Fermentation Protein,” filed Feb. 19, 2025, the disclosure of which is hereby incorporated herein by reference.

### FIELD

This disclosure relates to systems and methods for producing renewable alcohols by fermentative processes involving recombinant host cells that comprise selected promoters for differential gene expression during the propagation and production phases of the fermentative processes.

### DESCRIPTION OF THE TEXT FILE SUBMITTED ELECTRONICALLY

The contents of the Sequence Listing XML submitted electronically herewith are incorporated herein by reference in their entirety: A computer readable format copy of the Sequence Listing (filename: GEVO-607-WO-01_SeqList_ST26.xml, date created: Jun. 10, 2026, file size: 73,728 bytes).

### BACKGROUND

Alcohols are important industrial chemicals, useful as reagents, solvents, fuel additives, feedstock chemicals for plastics, and chemical intermediates. Renewable alocohols may be used in the production of transportation fuels such as gasoline, jet fuel, and diesel fuels. Accordingly, there is a high demand for alcohols (e.g., butanol) as well as for efficient and environmentally friendly methods of producing alcohols.

Production of alocohols using fermentation by microorganisms is one such environmentally friendly production method. For example, alcohol production methods using a synthetic biochemistry pathway for the conversion of a fermentable carbon source (e.g., a sugar) to alcohol are commonly known and may be used for converting glucose into isobutanol. However, in order to develop an economically competitive fermentation process, a number of factors, including the development of a robust microorganism (“biocatalyst”) that may produce the alcohol, carbon sources capable of being metabolized by the microorganism, and recovery of the alcohol from a fermentation broth, may all be considered. In particular, the fermentative production of alcohol by microorganisms may challenge or inhibit the health, growth, and/or propagation of the microorganisms, resulting in reduced alcohol titers.

The activation and/or continuous production of alcohol by fermentation requires a large number of resources from a microorganism, which functions as a cell factory. Alcohol production using the carbon source-to-alcohol synthetic biochemistry pathway involves the expression of synthetic pathway genes that encode synthetic proteins (e.g., enzymes), which participate in the conversion of sugar to alcohol. In fact, to achieve maximum alcohol titer, synthetic pathway proteins may account for up to 30% to 35% of the cellular proteins expressed during alcohol production in a microorganism. The expression of synthetic pathway proteins may thus place a significant burden on the microorganisms used for alcohol production, potentially leading to poor cell health and impaired growth. In addition, the microorganism may suffer from limited ability to respond to extracellular stresses such as osmotic pressure, pH balance, and the consumption regulation of glucose or nitrogen.

Thus, alcohol production may be limited in microorganism-cell-based fermentation due to the need to maintain cell viability while expressing synthetic pathway proteins. One option to address this limitation is regulating the expression of the genes encoding the pathway proteins. For example, the use of carbon-source-responsive promoters in synthetic pathway protein expression sequences, where the synthetic pathway is activated in the presence of fermentable carbon sources, is known. Recombinant host cells that comprise synthetic pathway protein expression sequences having carbon-source-responsive promoters exhibit a significant increase in the expression of pathway genes in the presence of a fermentable carbon source (e.g., corn mash feedstock). While this carbon-source-responsive regulation does prevent extraneous gene expression during propagation in the presence of non-fermentative carbon sources, it may also place a significant burden on cells at the beginning of fermentation and alcohol production, when the cells are first combined with a fermentable carbon source. The presence of the fermentable carbon source may trigger a significant induction of pathway gene expression, which may contribute to decreased cell growth, reduced cell division, and limited ability of cells to adapt to the various extracellular stressors encountered during fermentation (e.g., pH changes, osmotic pressure).

Thus, there is a continuing need to address the challenges of reduced cell health, growth, and/or propagation in microorganisms used for microbial fermentation and to develop more efficient methods and systems for producing alcohols such as butanol.

### SUMMARY

This disclosure describes, among other things, methods of producing renewable alcohol by microbial fermentation and compositions for producing renewable alcohol, particularly isobutanol, as well as methods for converting the renewable alcohol to fuel. The disclosure also describes selected promoters for differential gene expression during the propagation and production phases of the microbial fermentation processes, recombinant yeast cells that comprise such promoters, and methods for producing the recombinant yeast cells.

In some embodiments, a method for producing renewable alcohol includes: a) contacting a recombinant host cell comprising a heterologous polynucleotide with a carbon substrate to produce a reaction mixture, the heterologous polynucleotide comprising: i) a promoter nucleic acid sequence, wherein the promoter nucleic acid sequence has at least 90% identity to SEQ ID NO: 2, SEQ ID NO: 7, SEQ ID NO: 12, SEQ ID NO: 18, a functional fragment thereof, a variant thereof, or a derivative thereof; and ii) a nucleic acid sequence encoding a biocatalyst peptide operably linked to the promoter nucleic acid sequence; b) fermenting the reaction mixture under a first set of conditions and a second set of conditions to produce alcohol; and c) optionally recovering the renewable alcohol; wherein the second set of conditions differs from the first set of conditions, the nucleic acid sequence encoding the alcohol biosynthetic pathway enzyme is differentially expressed under the first set of conditions than under the second set of conditions, and the recombinant host cell produces alcohol under at least one of the first set of conditions or the second set of conditions. The renewable alcohol may be butanol, such as isobutanol. In some embodiments, the biocatalyst peptide may be an alcohol biosynthetic pathway enzyme selected from the group consisting of acetolactate synthase (ALS), ketol-acid reductoisomerase (KARI), dihydroxyacid dehydratase (DHAD), 2-keto-acid decarboxylase (KIVD), alcohol dehydrogenase (ADH), and combinations thereof. In certain embodiments, the biocatalyst polypeptide may include a sequence having at least 90% identity to SEQ ID NO: 22-26.

In some embodiments, a method for producing butanol includes: a) contacting a recombinant host cell comprising a heterologous polynucleotide with a carbon substrate to produce a reaction mixture, the heterologous polynucleotide comprising: i) a promoter nucleic acid sequence, wherein the promoter nucleic acid sequence has at least 90% identity to SEQ ID NO: 2, SEQ ID NO: 7, SEQ ID NO: 12, SEQ ID NO: 18, a functional fragment thereof, a variant thereof, or a derivative thereof; and ii) a nucleic acid sequence encoding an enzyme operably linked to the promoter nucleic acid sequence, wherein the enzyme is selected from the group consisting of acetolactate synthase (ALS), ketol-acid reductoisomerase (KARI), dihydroxyacid dehydratase (DHAD), 2-keto-acid decarboxylase (KIVD), alcohol dehydrogenase (ADH), and mixtures thereof; b) fermenting the reaction mixture under a first set of conditions and a second set of conditions to produce butanol; and c) optionally recovering the butanol; wherein the second set of conditions differs from the first set of conditions, the nucleic acid sequence encoding the enzyme is differentially expressed under the first set of conditions than under the second set of conditions, and the recombinant host cell produces butanol under at least one of the first set of conditions or the second set of conditions.

In some embodiments, one or more of the following features may be included in any feasible combination in any of the described methods. For example, the optional step of recovering the renewable alcohol or the butanol may include: i) combining the reaction mixture and the renewable alcohol or the butanol with an organic solvent, wherein the renewable alcohol or the butanol partitions into a non-aqueous organic solvent phase and the reaction mixture partitions into an aqueous phase; and ii) distilling the non-aqueous organic solvent phase to separate the renewable alcohol or the butanol. The renewable alcohol or the butanol may be recovered before the concentration of the renewable alcohol or the butanol reaches a level toxic to the recombinant host cell.

In some embodiments, the recombinant host cell is a recombinant yeast cell. The yeast cell may be selected from the group consisting of Saccharomyces, Kluyveromyces, Schizosaccharomyces, and combinations thereof.

In some embodiments, the first set of conditions and the second set of conditions may differ in at least one of a source of the carbon substrate, a concentration of dissolved oxygen, a temperature, a pH, or a concentration of renewable alcohol or butanol. In certain embodiments, the rate of renewable alcohol or butanol production is less under the first set of conditions than under the second set of conditions.

In some embodiments, the expression of the nucleic acid sequence encoding the biocatalyst peptide or the nucleic acid sequence encoding the enzyme is higher in a production phase of fermentation than in a propagation phase of fermentation.

In some embodiments, an isolated polynucleotide includes: a) a promoter nucleic acid sequence, wherein the promoter nucleic acid sequence has at least 90% identity to SEQ ID NO: 2, SEQ ID NO: 7, SEQ ID NO: 12, SEQ ID NO: 18, a functional fragment thereof, a variant thereof, or a derivative thereof; and b) a nucleic acid sequence encoding an alcohol biosynthetic pathway enzyme operably linked to the promoter nucleic acid sequence. In certain embodiments, a recombinant host cell includes the isolated polynucleotide. In some embodiments, the recombinant host cell is a recombinant microbial cell, such as a recombinant yeast cell, wherein the yeast is selected from the group consisting of Saccharomyces, Kluyveromyces, Schizosaccharomyces, and combinations thereof.

In some embodiments, a composition for producing a renewable alcohol includes the recombinant host cell that includes the isolated polynucleotide. In certain embodiments, a method of producing a renewable alcohol includes: a) mixing the composition for producing the renewable alcohol with a carbon substrate to produce a reaction mixture; b) fermenting the reaction mixture to produce the renewable alcohol; and, optionally, c) recovering the renewable alcohol. In some embodiments, a multiphase fermentation composition includes: a) an aqueous phase comprising the reaction mixture; and b) a non-aqueous organic solvent phase comprising the renewable alcohol and an organic solvent. In certain embodiments, the organic solvent is selected from the group consisting of phenetole, octanol, heptanol, oleyl alcohol, and mixtures thereof.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter of this disclosure are contemplated as being part of the embodiments disclosed herein. It should also be appreciated that terminology explicitly employed herein that also may appear in any disclosure incorporated by reference should be accorded a meaning most consistent with the particular concepts disclosed herein.

### BRIEF DESCRIPTION OF THE DRAWINGS

FIG. 1 illustrates a standard schematic of a gene cassette comprising a promoter sequence, a sequence encoding a protein of interest, and a terminator sequence;

FIG. 2 illustrates a plasmid used to introduce a gene cassette containing a selected promoter into a yeast cell to assess the activity of the selected promoter.

FIG. 3. illustrates a schematic of a standard bicistronic gene cassette used to stably and sequentially integrate multiple copies of selected genes and their promoters into a yeast genome until sufficient copies have been added to provide the desired protein production response.

FIG. 4 is a graph showing the isobutanol yield of a fermentative, recombinant yeast cell comprising a functional isobutanol biosynthetic pathway and a nucleic acid sequence encoding an isobutanol biosynthetic pathway enzyme operably linked to a promoter nucleic acid sequence.

FIG. 5 is a graph showing the rate of isobutanol production for the fermentative, recombinant yeast cells of FIG. 4.

FIG. 6 is a graph showing growth rates for the fermentative, recombinant yeast cells of FIG. 4.

FIG. 7 is a graph showing growth rates for various fermentative, recombinant yeast cells expressing a nucleic acid sequence encoding an isobutanol biosynthetic pathway enzyme operably linked to promoter sequences from URA3 sequence or the HSP26 sequence.

FIG. 8 is a graph showing the isobutanol yield of a fermentative, recombinant yeast cell comprising a nucleic acid sequence encoding an isobutanol biosynthetic pathway enzyme operably linked to different URA3 promoter nucleic acid sequences.

FIG. 9 is a graph showing the rate of isobutanol production for the fermentative, recombinant yeast cells of FIG. 8.

FIG. 10 is a graph showing the isobutanol yield of a fermentative, recombinant yeast cell comprising a nucleic acid sequence encoding an isobutanol biosynthetic pathway enzyme operably linked to different HSP26 promoter nucleic acid sequences.

FIG. 11 is a graph showing the rate of isobutanol production for the fermentative, recombinant yeast cells of FIG. 10.

FIG. 12 is a graph showing growth rates for various fermentative, recombinant yeast cells expressing a nucleic acid sequence encoding an isobutanol biosynthetic pathway enzyme operably linked to promoter sequences from the FIT3 sequence and the HXT6 sequence.

FIG. 13 is a graph showing the isobutanol yield of a fermentative, recombinant yeast cell comprising a nucleic acid sequence encoding an isobutanol biosynthetic pathway enzyme operably linked to different FIT3 promoter nucleic acid sequences.

FIG. 14 is a graph showing the rate of isobutanol production for the fermentative, recombinant yeast cells of FIG. 13.

FIG. 15 is a graph showing the isobutanol yield of a fermentative, recombinant yeast cell comprising a nucleic acid sequence encoding an isobutanol biosynthetic pathway enzyme operably linked to different HXT6 promoter nucleic acid sequences.

FIG. 16 is a graph showing the rate of isobutanol production for the fermentative, recombinant yeast cells of FIG. 15.

FIGS. 17A and 17B are graphs showing growth rates for various fermentative, recombinant yeast cells during propagation and production, respectively.

FIG. 18 is a graph showing the isobutanol yield for the fermentative, recombinant yeast cells of FIGS. 17A and 17B.

### DETAILED DESCRIPTION

Unless defined otherwise, all technical and scientific terms used herein have the same meaning as commonly understood by one of ordinary skill in the art to which this invention belongs. In case of conflict, the present application including the definitions will control. Also, unless otherwise required by context, singular terms shall include pluralities, and plural terms shall include the singular. All publications, patents and other references mentioned herein are incorporated by reference in their entireties for all purposes.

In order to further define this invention, the following terms and definitions are herein provided.

Reference throughout this specification to “some embodiments,” “one embodiment” or “an embodiment” means a particular feature, structure or characteristic described in connection with the embodiment is included in at least one embodiment. Thus, the appearances of the phrases “in some embodiments,” “in one embodiment” or “in an embodiment” in various places throughout this specification are not necessarily all referring to the same embodiment. Furthermore, the particular features, structures, or characteristics may be combined in any suitable manner in one or more embodiments. Also, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

The word “about” when immediately preceding a numerical value means a range of plus or minus 10% of that value, e.g., “about 50” means 45 to 55, “about 25,000” means 22,500 to 27,500. Furthermore, the phrases “less than about” a value or “greater than about” a value should be understood in view of the definition of the term “about” provided herein.

As used herein, the term “recombinant microorganism” refers to a microorganism, such as bacterium or yeast, that is modified by use of recombinant DNA techniques by, for example, engineering a host cell to comprise a biosynthetic pathway, such as a biosynthetic pathway to produce an alcohol (e.g., butanol).

As used herein, the term “biocatalyst polypeptide” includes biosynthetic pathway polypeptides that are associated with substrate-to-product conversions of an indicated biosynthetic pathway (e.g., alcohol biosynthetic pathway), as well as polypeptides associated with the viability of a biocatalyst that is associated with the indicated biosynthetic pathway, including cell integrity polypeptides and propagation polypeptides. For example, a polypeptide that is part of an NADPH generating pathway or a polypeptide that is part of a non-alcohol NADH consuming product pathway may be a biocatalyst polypeptide.

As used herein, the term “isolated,” as in an “isolated polypeptide” or “an isolated nucleic acid,” refers to a nucleic acid or polypeptide that is free or substantially free of other cellular components with which it is associated in the natural state, including a purified nucleic acid or polypeptide. No particular level of purification is required. For example, an isolated polypeptide or nucleic acid may be separated, fractionated, and/or partially or substantially purified by any suitable technique.

As used herein, a “fragment” is a portion or segment of a reference sequence. For example, a fragment of a promoter nucleic acid sequence or a nucleic acid sequence encoding a biocatalyst polypeptide is identical in sequence to a portion of the reference sequence and shorter in length than the reference sequence. A fragment may comprise from 5 to 1000 contiguous nucleotides. A fragment used as a probe, primer, or for other purposes, may be at least 5, at least 10, or at least 15, or at least 16, or at least 20, or at least 25, or at least 30, or at least 40, or at least 50, or at least 60, or at least 75, or at least 100, or at least 150, or at least 250, or at least 500 contiguous nucleotides. A fragment may comprise up to the entire length of the reference sequence minus one nucleotide or amino acid. Fragments may be preferentially selected from certain regions of a molecule. For example, a polynucleotide fragment may comprise a certain length of contiguous nucleotides selected from the first 100 or 200 nucleotides of a polynucleotide.

As used herein, an active or functional fragment is understood as a sequence fragment having a substantially similar function to a reference sequence. “Similar function” is understood to mean activity, for example promoter activity or enzyme activity (e.g., catalyzing the indicated substrate to product conversion). Substantially similar function of a functional fragment may be at about 50%, or at least about 60%, or at least about 70%, or at least about 80%, or at least about 90%, or at least about 95% activity compared to an activity of the reference sequence or protein.

As used herein, the term “polypeptide” encompasses a singular “polypeptide” as well as plural “polypeptides” and refers to a molecule composed of amino acid monomers linked together by peptide bonds. The term “polypeptide” refers to any chain or chains of two or more amino acids and is not limited to a particular chain length. Thus, peptides, dipeptides, tripeptides, oligopeptides, “protein,” “amino acid chain,” or any other term used to refer to a chain or chains of two or more amino acids are included within the definition of “polypeptide,” and the term “polypeptide” may be used interchangeably with any of these terms.

A polypeptide may comprise at least about 10, at least about 20, at least about 25, at least about 50, at least about 75, at least about 100, at least about 200, at least about 500, at least about 1,000, or at least about 2,000 amino acids. The term “polypeptide” also encompasses modified polypeptides, such as polypeptides that have been post-translationally modified. Post-translational modifications include glycosylation, acetylation, phosphorylation, amidation, derivatization by known protecting/blocking groups, proteolytic cleavage, or modification by non-naturally occurring amino acids. Polypeptides may or may not have a three-dimensional structure, referred to as folding or structural folding. The polypeptides described herein may comprise full-length polypeptides, active or functional fragments thereof, variants thereof, analogs thereof, and/or derivatives thereof.

Variants of polypeptides of the present invention include polypeptides with altered amino acid sequences due to amino acid substitutions, deletions, and/or insertions. Variants may occur naturally or be non-naturally occurring. Non-naturally occurring variants may be produced using known mutagenesis techniques. Variant polypeptides may comprise conservative or non-conservative amino acid substitutions, deletions, and/or additions. Derivative polypeptides include polypeptides that have been altered to exhibit additional features not found in the native or wild-type polypeptide, such as fusion proteins. Variant polypeptides may also be referred to herein as “polypeptide analogs.” Derivative polypeptides include polypeptides that have one or more amino acid residues chemically derivatized by reaction of a functional side group and polypeptides that contain one or more naturally occurring amino acid derivatives of the twenty standard amino acids (e.g., 4-hydroxyproline, 5-hydroxylysine, 3-methylhistidine, homoserine).

The polypeptides described herein may include biosynthetic pathway polypeptides or enzymes, cell integrity polypeptides or enzymes, propagation polypeptides or enzymes, and other polypeptides or enzymes.

The term “promoter” refers to a nucleic acid sequence (such as DNA) capable of controlling the expression of a coding sequence or functional RNA. In general, a coding sequence is located 3′ to a promoter sequence and begins with a start codon having the nucleic acid sequence ATG. Promoters may be derived in their entirety from a native gene or be composed of different elements derived from different promoters found in nature, or even comprise synthetic nucleic acid segments. It is understood by those skilled in the art that different promoters may direct the expression of a gene in different tissues or cell types, or at different stages of development, or in response to different environmental or physiological conditions. Promoters that cause a gene to be expressed in most cell types at most times are commonly referred to as “constitutive promoters.” “Inducible promoters,” on the other hand, cause a gene to be expressed when the promoter is induced or turned on by a promoter-specific signal or molecule. It is further recognized that since in most cases the exact boundaries of regulatory sequences have not been completely defined, DNA fragments of different lengths may have identical promoter activity. For example, it will be understood that “FBA1 promoter” may be used to refer to a fragment derived from the promoter region of the FBA1 gene.

As used herein, the term “PSc” denotes a Saccharomyces cerevisiae promoter.

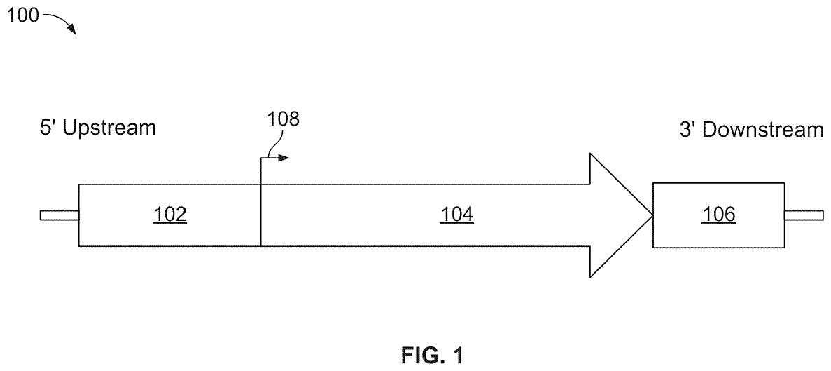

As used herein, the terms “expression construct” or “gene cassette” comprise a promoter nucleic acid sequence operably linked to a coding region for a polypeptide and, optionally, a terminator nucleic acid sequence. For example, FIG. 1 displays a basic schematic of a DNA sequence 100 that is a gene cassette comprising three basic elements: a promoter region 102 (e.g., PSCURA3), a protein coding sequence 104 (e.g., KARI), and a terminator sequence 106 (e.g., TScCYC1). The activation of the promoter 102 is accomplished by the binding of DNA binding proteins that recruit RNA-polymerase to transcribe the protein coding sequence 104 into mRNA molecules, starting at the start codon 108. Promoter activity is primarily driven by the unique sequence identity and DNA structure of the promoter, which in turn can lead to more, or less, RNA polymerase activity.

As used herein, the term “fermentable carbon source” refers to a carbon source capable of being metabolized by microorganisms (such as the microorganisms disclosed herein) for the production of alcohol. Suitable fermentable carbon sources include, but are not limited to, monosaccharides such as glucose or fructose; disaccharides such as lactose or sucrose; oligosaccharides; polysaccharides such as starch or cellulose; C₈ sugars such as xylose or arabinose; carbon substrates such as methane; and mixtures thereof. The terms “fermentable carbon source,” “carbon substrate,” and “fermentable carbon substrate” are used interchangeably.

As used herein, the term “feedstock” refers to a feed in a fermentation process, the feed containing a fermentable carbon source with or without undissolved solids, and where applicable, the feed containing the fermentable carbon source before or after the fermentable carbon source has been liberated from starch or obtained from the breakdown of complex sugars by further processing, such as by liquefaction, saccharification, or other processes. Suitable feedstocks include, but are not limited to, rye, wheat, barley, corn, corn mash, cane, cane mash, cellulosic material, lignocellulosic material, and mixtures thereof.

As used herein, the terms “fermentation broth,” “fermentation liquid,” “fermentation medium,” and “fermented mixture” are used interchangeably and refer to a mixture containing, among other constituents, fermentative microorganisms, fermentable carbon sources (e.g., glucose), and alcohol.

As used herein, the terms “microorganism biomass” or “yeast biomass” refer to the cell biomass of the fermentation product-producing microorganism or yeast, typically provided in units g/L dry cell weight (dcw).

As used herein, the terms “growth phase” and “propagation phase” refer to the process steps during which yeast biomass is produced and inoculum build-up occurs. The growth or propagation phase is generally performed on a non-fermentable carbon source including, but not limited to, ethanol and glycerol substrates, or corn thin stillage. Typically, this process does not produce isobutanol.

The term “production phase” refers to the fermentation process steps during which the production of a desired fermentation product, including, but not limited to, butanol, isobutanol, 1-butanol, 2-butanol, and/or 2-butanone, occurs. The production phase generally begins upon the addition of the yeast to a fermentable carbon source.

Disclosed herein is a process for producing renewable alcohol using microbial fermentation. Renewable alcohols include, but are not limited to, C₁ to C₈ alkyl alcohols, isomers of C₁ to C₈ alkyl alcohols, and mixtures thereof. In some embodiments, the alcohols are C₂ to C₈ alkyl alcohols. In some embodiments, the alcohols are C₂ to C₈ alkyl alcohols or C₃ to C₆ alkyl alcohols. It will be appreciated that C₁ to C₈ alkyl alcohols include, but are not limited to, methanol, ethanol, propanol, butanol, pentanol, and hexanol. C₂ to C₈ alkyl alcohols include, but are not limited to, ethanol, propanol, butanol, and pentanol.

### Fermentative Recombinant Microorganism

The process for producing renewable alcohol described herein may use any alcohol-producing microorganism, including recombinant alcohol-producing microorganisms. Though many native or naturally occurring alcohol-producing microorganisms (e.g., bacteria, yeast) exist, such native microorganisms may not produce alcohol at a yield sufficient for commercialization. For example, Clostridium acetobutylicum and other Clostridia are known to produce butanol via fermentation. In fact, acetone-butanol-ethanol (ABE) fermentation by Clostridium acetobutylicum is one of the oldest known industrial fermentations (as are also the pathways and genes responsible for the production of the acetone, butanol, and ethanol). Production of butanol by the ABE process, however, is limited by the toxic effect of the butanol on Clostridium acetobutylicum and by the generation of co-products, such as acetone and ethanol. Genetic modification using known molecular biological techniques may be used to develop recombinant microorganisms that fermentatively produce alcohol, including butanol, at higher yields. Suitable microorganisms that may be genetically modified include bacteria, cyanobacteria, filamentous fungi, and yeast.

Recombinant microorganisms may be engineered to express a selected metabolic pathway and/or to produce a desired metabolite to reduce or eliminate the production of undesired co-products, and/or to otherwise increase a yield of the desired metabolite. Metabolites include starting materials (e.g., glucose or pyruvate), intermediates (e.g., 2-ketoisovalerate, acetaldehyde), and end products (e.g., ethanol, butanol, other alcohols) of a selected metabolic pathway. Metabolites may be used to construct more complex molecules or be broken down into simpler molecules. Intermediate metabolites may be synthesized from other metabolites, used to make more complex molecules, or broken down into simpler molecules, often with the release of chemical energy. Examples of metabolites include glucose, pyruvate, and alcohol, such as butanol.

In order to develop an economically viable microbial fermentation process, a number of factors, such as the development or identification of a robust recombinant microorganism (“biocatalyst”) that may produce the alcohol, the identification of carbon sources capable of being metabolized by the microorganism, the efficient recovery of the alcohol from a fermentation broth, co-product formation, and the potential for contamination, may be considered. In particular, as discussed above, because the fermentative production of alcohol requires a significant amount of resources from a biocatalyst, such as yeast, biocatalyst viability may be adversely impacted. Specifically, the expression of synthetic alcohol production genes and proteins may place a significant burden on biocatalysts, potentially leading to poor cell health, greatly impaired growth, and limited ability of the cell to respond to extracellular stresses. Thus, alcohol production may be limited in microorganism-cell-based fermentation due to the need to maintain cell viability while expressing synthetic pathway proteins.

As discussed above, this limitation may be addressed by regulating the expression of genes encoding alcohol production proteins. In particular, by identifying select promoter nucleic acid sequences that may provide differential expression of genes of interest under different conditions and by providing biocatalysts that are genetically modified to include such promoter nucleic acid sequences, differential expression during biocatalyst propagation and alcohol production phases may be achieved. The nucleic acid sequences described herein may be employed as promoters for the expression of various polypeptides relevant to biocatalyst propagation and/or alcohol production and may be referred to as “genetic switches.”

By using biocatalysts that are genetically modified to include the promoter nucleic acid sequences described herein, the expression of alcohol production genes may be shifted from propagation phase or early production phase to mid- to late-production phase to allow cells to grow and divide during early stages of production. By regulating the expression of pathway genes in this way, microorganism biomass may increase early in the fermentation process, which may lead to increased alcohol titers and greater sugar consumption efficiency. Biocatalysts that include the promoter nucleic acid sequences described herein may also avoid the inherent decrease in pathway enzyme activity associated with later stages of the fermentation process (as a result of lowered sugar concentrations) and maintain a suitable concentration of pathway enzymes, which may increase product alcohol titers and decrease the production of undesired co-products.

The expression of selected metabolic pathway genes and/or genes encoding polypeptides that produce a desired metabolite, such as alcohol, may be controlled by introducing a promoter sequence into the gene of interest. A polynucleotide comprising a nucleic acid, such as DNA, that encodes a polypeptide may include a promoter and/or other transcription control elements, such as terminator sequences, operably linked or associated with one or more coding regions. The terms “operably linked,” “operably associated,” or “coupled,” as used herein, refers to a functional linkage between a promoter and a second sequence, wherein the promoter sequence initiates and mediates transcription of the DNA sequence corresponding to the second sequence. Operably linked nucleic acid sequences may be contiguous and/or may join two protein coding regions in the same reading frame. For example, a coding region for a gene product, e.g., a polypeptide, may be operably associated with one or more regulatory sequences such that expression of the gene product is under the influence or control of the regulatory sequence(s). Other transcription control elements besides a promoter, for example enhancers, operators, repressors, and transcription termination signals, may be operably associated with the polynucleotide. Suitable promoters and/or other transcription control regions are disclosed herein.

Initiation control regions or promoters that are useful to drive expression of the relevant pathway coding regions in yeast cells are numerous and known to those skilled in the art. Promoters useful for expression in yeast cells include, but are not limited to, the following: CYC1, FBA1, HIS3, GAL1, GAL10, ILV5, ADH1, PDC1, PGK, PHO5, GAPDH, GPM1, ADC1, TDH3 (GPD), TRP1, LEU2, ENO, OLE1, CUP1, and TPI. FBA1, TDH3 (GPD), ADH1, ILV5, and GPM1 are constitutive promoters, while GAL1, GAL10, OLE1, and CUP1 inducible promoters. Other yeast promoters include hybrid promoters, such as UAS(PGK1)-FBA1p, UAS(PGK1)-ENO1p, UAS(FBA1)-PDC1p), UAS(PGK1)-PDC1p, and UAS(PGK)-OLE1p, described in U.S. patent application Ser. No. 13/428,585, filed Mar. 23, 2012, incorporated herein by reference in its entirety.

Some promoter nucleic acid sequences disclosed herein, including those in Table 1, were taken to be 1000 bp 5′ of the start codon of each gene. However, the sequences may be retrieved from publicly available databases such as the Yeastract database (at www.yeastract.com) or the Saccharomyces Genome Database (SGD) (at www.yeastgenome.org). The gene name is indicated. It should be understood by those skilled in the art that fragments of different lengths of the sequences provided may have identical promoter activity. For example, the “HSP26 promoter” should be understood to encompass a sequence provided herein or any fragment of the promoter region of the HSP26 gene that has a substantially similar or identical promoter activity, a substantially similar effect on expression of a target polypeptide, or a substantially similar effect on production of an indicated product. Thus, the disclosed nucleic acid sequences should not be construed as limited solely to the provided sequence.

The promoter may be active or more active at certain phases or stages of alcohol, particularly isobutanol, fermentation. In some embodiments, the promoter activity may provide for differential expression in propagation and production stages of fermentation, particularly differential expression in propagation and in early production versus in late production. In certain embodiments, the promoter is active after about 8 to about 32 hours, or about 10 to about 30 hours, or about 16 to about 24 hours of alcohol, particularly isobutanol, fermentation. In some embodiments, the promoter is inactive or has low activity at certain phases or stages of alcohol, particularly isobutanol, fermentation, such as during propagation and early production. In certain embodiments, the promoter is inactive or has low activity from the beginning of fermentation until after about 6 hours, or about 8 hours, or about 10 hours, or about 12 hours, or about 14 hours of alcohol, particularly isobutanol, fermentation.

In some aspects, the promoter activity is sensitive to one or more physiochemical differences between propagation and production stages of fermentation. In some examples, the promoter activity is sensitive to the dissolved oxygen concentration, the glucose concentration of the medium, the glucose concentration within the cell, the source of the fermentable carbon substrate, the concentration of essential nutrients in the medium-such as nucleic acids or amino acids-, the concentration of butanol in fermentation medium, the pH in the fermentation medium, and/or the temperature in the fermentation medium.

Methods for evaluating promoter nucleic acid sequences and identifying promoter sequences that may be active or more active at certain phases or stages of fermentation (e.g., preferential increase or decrease gene expression under certain conditions) may include RNA transcript comparison between microbial cells grown under selected propagation conditions and cells grown under selected production conditions. Suitable promoters may be associated with RNA transcripts that are upregulated or downregulated during the propagation phase as compared to during the production phase. Promoters associated with RNA transcripts that are upregulated or downregulated during the production phase as compared to the propagation phase may also be suitable. Promoters identified from RNA transcript comparison may be further characterized by their nucleic acid content. For instance, AT-rich regions are likely promoter regions.

In some examples, the promoter nucleic acid sequence comprises a PSCURA3 sequence having at least about 70%, or at least about 80%, or at least about 85%, or at least about 90%, or at least about 95%, or at least about 96%, or at least about 97%, or at least about 98%, or at least about 99%, or about 100% identity to any one of SEQ ID NOs: 1-20, a functional fragment thereof, a variant thereof, or a derivative thereof.

In some examples, the promoter nucleic acid sequence comprises a PSCURA3 sequence having at least about 70%, or at least about 80%, or at least about 85%, or at least about 90%, or at least about 95%, or at least about 96%, or at least about 97%, or at least about 98%, or at least about 99%, or about 100% identity to any one of SEQ ID NOs: 1-4, a functional fragment thereof, a variant thereof, or a derivative thereof.

In certain examples, the promoter nucleic acid sequence comprises a PScHSP26 sequence having at least about 70%, or at least about 80%, or at least about 85%, or at least about 90%, or at least about 95%, or at least about 96%, or at least about 97%, or at least about 98%, or at least about 99%, or about 100% identity to any one of SEQ ID NOs: 5-9, a functional fragment thereof, a variant thereof, or a derivative thereof.

In further examples, the promoter nucleic acid sequence comprises a PScFIT3 sequence having at least about 70%, or at least about 80%, or at least about 85%, or at least about 90%, or at least about 95%, or at least about 96%, or at least about 97%, or at least about 98%, or at least about 99%, or about 100% identity to any one of SEQ ID NOs: 10-15, a functional fragment thereof, a variant thereof, or a derivative thereof.

In still further examples, the promoter nucleic acid sequence comprises a PSCHXT6 sequence having at least about 70%, or at least about 80%, or at least about 85%, or at least about 90%, or at least about 95%, or at least about 96%, or at least about 97%, or at least about 98%, or at least about 99%, or about 100% identity to any one of SEQ ID NOs: 17-20, a functional fragment thereof, a variant thereof, or a derivative thereof.

In some examples, the promoter nucleic acid sequence comprises a PSCURA3 sequence having at least about 70%, or at least about 80%, or at least about 85%, or at least about 90%, or at least about 95%, or at least about 96%, or at least about 97%, or at least about 98%, or at least about 99%, or about 100% identity to SEQ ID NO: 2, a functional fragment thereof, a variant thereof, or a derivative thereof.

In further examples, the promoter nucleic acid sequence comprises a PSCURA3 sequence having at least about 70%, or at least about 80%, or at least about 85%, or at least about 90%, or at least about 95%, or at least about 96%, or at least about 97%, or at least about 98%, or at least about 99%, or about 100% identity to SEQ ID NO: 4, a functional fragment thereof, a variant thereof, or a derivative thereof.

In some examples, the promoter nucleic acid sequence comprises a PScHSP26 sequence having at least about 70%, or at least about 80%, or at least about 85%, or at least about 90%, or at least about 95%, or at least about 96%, or at least about 97%, or at least about 98%, or at least about 99%, or about 100% identity to SEQ ID NO: 7, a functional fragment thereof, a variant thereof, or a derivative thereof.

In further examples, the promoter nucleic acid sequence comprises a PScHSP26 sequence having at least about 70%, or at least about 80%, or at least about 85%, or at least about 90%, or at least about 95%, or at least about 96%, or at least about 97%, or at least about 98%, or at least about 99%, or about 100% identity to SEQ ID NO: 9, a functional fragment thereof, a variant thereof, or a derivative thereof.

In some examples, the promoter nucleic acid sequence comprises a PScFIT3 sequence having at least about 70%, or at least about 80%, or at least about 85%, or at least about 90%, or at least about 95%, or at least about 96%, or at least about 97%, or at least about 98%, or at least about 99%, or about 100% identity to SEQ ID NO: 12, a functional fragment thereof, a variant thereof, or a derivative thereof.

In further examples, the promoter nucleic acid sequence comprises a PScFIT3 sequence having at least about 70%, or at least about 80%, or at least about 85%, or at least about 90%, or at least about 95%, or at least about 96%, or at least about 97%, or at least about 98%, or at least about 99%, or about 100% identity to SEQ ID NO: 15, a functional fragment thereof, a variant thereof, or a derivative thereof.

In some examples, the promoter nucleic acid sequence comprises a PSCHXT6 sequence having at least about 70%, or at least about 80%, or at least about 85%, or at least about 90%, or at least about 95%, or at least about 96%, or at least about 97%, or at least about 98%, or at least about 99%, or about 100% identity to SEQ ID NO: 18, a functional fragment thereof, a variant thereof, or a derivative thereof.

In further examples, the promoter nucleic acid sequence comprises a PSCHXT6 sequence having at least about 70%, or at least about 80%, or at least about 85%, or at least about 90%, or at least about 95%, or at least about 96%, or at least about 97%, or at least about 98%, or at least about 99%, or about 100% identity to SEQ ID NO: 20, a functional fragment thereof, a variant thereof, or a derivative thereof.

The term “percent identity,” as known in the art, is a relationship between two or more polypeptide sequences or two or more polynucleotide sequences, as determined by comparing the sequences. “Identity” may also refer to the degree of sequence relatedness between polypeptide or polynucleotide sequences, as determined by the match between strings of such sequences. “Identity” and “similarity” may be readily calculated by known methods, including but not limited to those disclosed in Computational Molecular Biology (Lesk, A. M., Ed.) Oxford University: NY (1988); Biocomputing: Informatics and Genome Projects (Smith, D. W., Ed.) Academic: NY (1993); Computer Analysis of Sequence Data, Part I (Griffin, A. M., and Griffin, H. G., Eds.) Humania: NJ (1994); Sequence Analysis in Molecular Biology (von Heinje, G., Ed.) Academic (1987); and Sequence Analysis Primer (Gribskov, M. and Devereux, J., Eds.) Stockton: NY (1991).

Tables 1 and 2 display the nucleotide sequences constituting SEQ ID NO's 1-31.

TABLE 1

| SEQ ID | | | |
|---|---|---|---|
| NO: | Nucleotide Sequence | | Description |
| SEQ ID | 1 | TCATAAAATT GATAAGGAGA ATCCATACAA GAAATCGATC GAATTATCAT TGAAATCCAT | PSCURA3- |
| NO: 1 | 61 | TCAACTAACA TCACACTTGC TGGAAGATAA TAACGATCTG CGTAAAAACG AGATATTCGC | 1000 |
| | 121 | TATTATTCAA GCTTTGGCAC ATCAATGCAT CAATCCGTGT AAGCAGATAA GTGAATTTGC | SGD ID |
| | 181 | AGTGGTAACG CTAGAGCAGA CGCTCATCAA TAAAATCGAA ATTCCAACTA ATGAGATGGA | YEL021W |
| | 241 | ATCGGTAGAA GAATTAATTG AGGGCGGATT ACTACCGTTG CTAAATTCGA GTGAAACACA | |
| | 301 | GGAAGACCAG AAAATCCTCA TTTCATCCAT ATTAACAATA ATTTCAAATG TTTATTTGCA | |
| | 361 | TTATTTGAAA CTAGGGAAGA CAAGCAACGA AACGTTTTTG AAAATTTTGA GTATTTTCAA | |
| | 421 | TAAATTTGTA GAGGACTCAG ATATTGAAAA AAAGCTACAG CAATTAATAC TTGATAAGAA | |
| | 481 | GAGTATTGAG AAGGGCAACG GTTCATCATC TCATGGATCT GCACATGAAC AAACACCAGA | |
| | 541 | GTCAAACGAC GTTGAAATTG AGGCTACTGC GCCAATTGAT GACAATACAG ACGATGATAA | |
| | 601 | CAAACCGAAG TTATCTGATG TAGAAAAGGA TTAAAGATGC TAAGAGATAG TGATGATATT | |
| | 661 | TCATAAATAA TGTAATTCTA TATATGTTAA TTACCTTTTT TGCGAGGCAT ATTTATGGTG | |
| | 721 | AAGGATAAGT TTTGACCATC AAAGAAGGTT AATGTGGCTG TGGTTTCAGG GTCCATAAAG | |
| | 781 | CTTTTCAATT CATCTTTTTT TTTTTTGTTC TTTTTTTTGA TTCCGGTTTC TTTGAAATTT | |
| | 841 | TTTTGATTCG GTAATCTCCG AGCAGAAGGA AGAACGAAGG AAGGAGCACA GACTTAGATT | |
| | 901 | GGTATATATA CGCATATGTG GTGTTGAAGA AACATGAAAT TGCCCAGTAT TCTTAACCCA | |
| | 961 | ACTGCACAGA ACAAAAACCT GCAGGAAACG AAGATAAATC | |
| SEQ ID | 1 | GCAATTAATA CTTGATAAGA AGAGTATTGA GAAGGGCAAC GGTTCATCAT CTCATGGATC | PSCURA3-541 |
| NO: 2 | 61 | TGCACATGAA CAAACACCAG AGTCAAACGA CGTTGAAATT GAGGCTACTG CGCCAATTGA | |
| | 121 | TGACAATACA GACGATGATA ACAAACCGAA GTTATCTGAT GTAGAAAAGG ATTAAAGATG | |
| | 181 | CTAAGAGATA GTGATGATAT TTCATAAATA ATGTAATTCT ATATATGTTA ATTACCTTTT | |
| | 241 | TTGCGAGGCA TATTTATGGT GAAGGATAAG TTTTGACCAT CAAAGAAGGT TAATGTGGCT | |
| | 301 | GTGGTTTCAG GGTCCATAAA GCTTTTCAAT TCATCTTTTT TTTTTTTGTT CTTTTTTTTG | |
| | 361 | ATTCCGGTTT CTTTGAAATT TTTTTGATTC GGTAATCTCC GAGCAGAAGG AAGAACGAAG | |
| | 421 | GAAGGAGCAC AGACTTAGAT TGGTATATAT ACGCATATGT GGTGTTGAAG AAACATGAAA | |
| | 481 | TTGCCCAGTA TTCTTAACCC AACTGCACAG AACAAAAACC TGCAGGAAAC GAAGATAAAT | |
| | 541 | C | |
| SEQ ID | 1 | GTGATGATAT TTCATAAATA ATGTAATTCT ATATATGTTA ATTACCTTTT TTGCGAGGCA | PSCURA3-351 |
| NO: 3 | 61 | TATTTATGGT GAAGGATAAG TTTTGACCAT CAAAGAAGGT TAATGTGGCT GTGGTTTCAG | |
| | 121 | GGTCCATAAA GCTTTTCAAT TCATCTTTTT TTTTTTTGTT CTTTTTTTTG ATTCCGGTTT | |
| | 181 | CTTTGAAATT TTTTTGATTC GGTAATCTCC GAGCAGAAGG AAGAACGAAG GAAGGAGCAC | |
| | 241 | AGACTTAGAT TGGTATATAT ACGCATATGT GGTGTTGAAG AAACATGAAA TTGCCCAGTA | |
| | 301 | TTCTTAACCC AACTGCACAG AACAAAAACC TGCAGGAAAC GAAGATAAAT C | |
| SEQ ID | 1 | CATAAAGCTT TTCAATTCAT CTTTTTTTTT TTTGTTCTTT TTTTTGATTC CGGTTTCTTT | PSCURA3-227 |
| NO: 4 | 61 | GAAATTTTTT TGATTCGGTA ATCTCCGAGC AGAAGGAAGA ACGAAGGAAG GAGCACAGAC | |
| | 121 | TTAGATTGGT ATATATACGC ATATGTGGTG TTGAAGAAAC ATGAAATTGC CCAGTATTCT | |
| | 181 | TAACCCAACT GCACAGAACA AAAACCTGCA GGAAACGAAG ATAAATC | |
| SEQ ID | 1 | CGTTGGACTT TTTTTAATAT AACCTACCAT AGGACACACT TTGTTGTTGA TGTTGGACAA | PScHSP26- |
| NO: 5 | 61 | TTCGTTAATT AAGAGTCCCT AAACGGCTCT ACTAGTTCCA ACCTCACTTT GTTTTTTCAT | 1000 |
| | 121 | TTTTTTATGT TTTTTCTAGA ACCTTCTTTA CGTGATTCTC GCTCGGAATC CGTCAATAGA | SGD ID |
| | 181 | ATGTTTTCAG TCTCCGTTTC AATATTCTGC GCACATCAAT CATTTTCTTA CTACATACAC | YBR072W |
| | 241 | TAACATTACT CCTAGTTTAA TTTAATTGAA TTTTTAACTT TCTTTTCTTT TCATTTGGCA | |
| | 301 | ATTTGGCTCC TTGAAAACAA GACTATGGGT CTCTCTCATA AGCCTCAGGG GGGGACCCCA | |
| | 361 | AAAAAATAAC GCGGCCATCT TGCATGCACC GTTGAACCTG TAGCTTACAG TAAGCCACAA | |
| | 421 | TTCTCTTACC TTCTTGGCAA TGTGGCACAA AATAATCTGG TTATGTGTCT TCATTTGGTA | |
| | 481 | ATCACTGGGA TGTTACTGGG GCAGCAGCAA CTCCGTGTGT ACCCCTAACT CCGTGTGTAC | |
| | 541 | CCCTAAAGAA CCTTGCCTGT CAAGGTGCAT TGTTGGATCG GAATAGTAAC CGTCTTTACA | |
| | 601 | TGAACATCCA CAACCAACGA AAGTGCTTTT TCAAGCATTG CTTGATTTCT AGAAAGATCG | |
| | 661 | ATGGTTATTC CCTCCCCCTT ATGCGTCCAA AAATATAGGG TGCTCGTAAC AGTAAGGTAT | |
| | 721 | TCGCACTTAG CGTGCTCGCA ACACAAAATT AAGTAATATG CGAGTTTTAG ATGTCCTTGC | |
| | 781 | GGATCTATGC ACGTTCTTGA GTGGTATTTC ATAACAACGG TTCTTTTTCA CCCTTATTCC | |
| | 841 | TAAACATATA AATAGGACCT CCATTAGTTA GAGATCTGTT TTTAATCCAT TCACCTTTCA | |
| | 901 | TTCTACTCTC TTATACTAAT AAAACCACCG ATAAAGATAT ATCAGATCTC TATTAAAACA | |
| | 961 | GGTATCCAAA AAAGCAAACA AACAAACTAA ACAAATTAAC | |
| SEQ ID | 1 | TTTGGCTCCT TGAAAACAAG ACTATGGGTC TCTCTCATAA GCCTCAGGGG GGGACCCCAA | PScHSP26- |
| NO: 6 | 61 | AAAAATAACG CGGCCATCTT GCATGCACCG TTGAACCTGT AGCTTACAGT AAGCCACAAT | 699 |
| | 121 | TCTCTTACCT TCTTGGCAAT GTGGCACAAA ATAATCTGGT TATGTGTCTT CATTTGGTAA | |
| | 181 | TCACTGGGAT GTTACTGGGG CAGCAGCAAC TCCGTGTGTA CCCCTAACTC CGTGTGTACC | |
| | 241 | CCTAAAGAAC CTTGCCTGTC AAGGTGCATT GTTGGATCGG AATAGTAACC GTCTTTACAT | |
| | 301 | GAACATCCAC AACCAACGAA AGTGCTTTTT CAAGCATTGC TTGATTTCTA GAAAGATCGA | |
| | 361 | TGGTTATTCC CTCCCCCTTA TGCGTCCAAA AATATAGGGT GCTCGTAACA GTAAGGTATT | |
| | 421 | CGCACTTAGC GTGCTCGCAA CACAAAATTA AGTAATATGC GAGTTTTAGA TGTCCTTGCG | |
| | 481 | GATCTATGCA CGTTCTTGAG TGGTATTTCA TAACAACGGT TCTTTTTCAC CCTTATTCCT | |
| | 541 | AAACATATAA ATAGGACCTC CATTAGTTAG AGATCTGTTT TTAATCCATT CACCTTTCAT | |
| | 601 | TCTACTCTCT TATACTAATA AAACCACCGA TAAAGATATA TCAGATCTCT ATTAAAACAG | |
| | 661 | GTATCCAAAA AAGCAAACAA ACAAACTAAA CAAATTAAC | |
| SEQ ID | 1 | GGATGTTACT GGGGCAGCAG CAACTCCGTG TGTACCCCTA AAGAACCTTG CCTGTCAAGG | PScHSP26- |
| NO: 7 | 61 | TGCATTGTTG GATCGGAATA GTAACCGTCT TTACATGAAC ATCCACAACC AACGAAAGTG | 495 |
| | 121 | CTTTTTCAAG CATTGCTTGA TTTCTAGAAA GATCGATGGT TATTCCCTCC CCCTTATGCG | |
| | 181 | TCCAAAAATA TAGGGTGCTC GTAACAGTAA GGTATTCGCA CTTAGCGTGC TCGCAACACA | |
| | 241 | AAATTAAGTA ATATGCGAGT TTTAGATGTC CTTGCGGATC TATGCACGTT CTTGAGTGGT | |
| | 301 | ATTTCATAAC AACGGTTCTT TTTCACCCTT ATTCCCAAAC ATATAAATAG GACCTCCATT | |
| | 361 | AGTTAGAGAT CTGTTTTTAA TCCATTCACC TTTCATTCTA CTCTCTTATA CTAATAAAAC | |
| | 421 | CACCGATAAA GATATATCAG ATCTCTATTA AAACAGGTAT CCAAAAAAGC AAACAAACAA | |
| | 481 | ACTAAACAAA TTAAC | |
| SEQ ID | 1 | ATTTCTAGAA AGATCGATGG TTATTCCCTC CCCCTTATGC GTCCAAAAAT ATAGGGTGCT | PScHSP26- |
| NO: 8 | 61 | CGTAACAGTA AGGTATTCGC ACTTAGCGTG CTCGCAACAC AAAATTAAGT AATATGCGAG | 356 |
| | 121 | TTTTAGATGT CCTTGCGGAT CTATGCACGT TCTTGAGTGG TATTTCATAA CAACGGTTCT | |
| | 181 | TTTTCACCCT TATTCCCAAA CATATAAATA GGACCTCCAT TAGTTAGAGA TCTGTTTTTA | |
| | 241 | ATCCATTCAC CTTTCATTCT ACTCTCTTAT ACTAATAAAA CCACCGATAA AGATATATCA | |
| | 301 | GATCTCTATT AAAACAGGTA TCCAAAAAAG CAAACAAACA AACTAAACAA ATTAAC | |
| SEQ ID | 1 | CCTTGCGGAT CTATGCACGT TCTTGAGTGG TATTTCATAA CAACGGTTCT TTTTCACCCT | PScHSP26- |
| NO: 9 | 61 | TATTCCCAAA CATATAAATA GGACCTCCAT TAGTTAGAGA TCTGTTTTTA ATCCATTCAC | 226 |
| | 121 | CTTTCATTCT ACTCTCTTAT ACTAATAAAA CCACCGATAA AGATATATCA GATCTCTATT | |
| | 181 | AAAACAGGTA TCCAAAAAAG CAAACAAACA AACTAAACAA ATTAAC | |
| SEQ ID | 1 | TCCATAAACA TTTCCTTTGT CTATATTATC CAGGGGCCCA AAATAAGCCC ATACCCACCT | PScFIT3- |
| NO: 10 | 61 | ATCTAGAGAG GCTTTATTGG GAGTGACGGG ATATCGTATG TTTTCCTTTA CCTCATCAAG | 1000 |
| | 121 | AGGCCGGATG AAAAGGGATG CATTGGCAAA AATTCGATAA TACTCCTCAT TGCTGACGAC | SGD ID |
| | 181 | CTCATCTTTG TGATAATCGT GGCAATACTG ATTAATTTTG CTAAACGTTT TTTCGAATAT | YOR383C |
| | 241 | TTTTTTACTT TTCTTCCTAC TGTCGAGAAC ATCCTTCGCG CAATGTAACC AAGACCCTAG | |
| | 301 | TGCTGGTTCA TAAGTGCAAA GAGTAGCGTG CCGACCCTTC ACATCCGTGT CAAATTTGTG | |
| | 361 | TGATTCCAAT TCTTTAGCAC AAGCATCAAT TGCTATCTGG TCCCATTGCG TTCTTTTCTT | |
| | 421 | AGTTGATGCT GGTTTTGCTA AAGAACCTGG TGCCAAATAC ACCAACAGCA GCACTAATCT | |
| | 481 | AGCGAAAAGC ATTCGAAATC TGGGTGAATT AAATTAACTG CGACAAAATT GGTGATGTGA | |
| | 541 | CATACCGAAA ACGCTACATT ACACATTTGC CAAGTAAAAA ATTGACGCTC CTTTTTTAAT | |
| | 601 | GATCGCTAAT GTAATGAAAA GTAAAAATCC AATTAAACGA GGAAGCGGTA AAATATGATT | |
| | 661 | GCTGTGTAGT AATGCCACAG TCTTCTTAGT CCTACCTATG TCCTAGATTT TTTTTTGCAC | |
| | 721 | CCTGTTTGTT CCGCACCCCG CTATCCCATA TTTTTTGATG TATGTGAAGG AAAGCAATCT | |
| | 781 | AAGGGCTCAA GAGCCGAGTT CCGCCATTCT AGGAAAGGGT GCAAAAAAAT GGGGTTCAAT | |
| | 841 | TATCGTTCGA GATTTGTTAT TTTTTTTTTT GTAAAAGTTG ATTTATGGGG TATTTAAGCC | |
| | 901 | AAGAACTATT TGAAGTATTA GGAGGTAATA TGAATGTTCG AACAGATACA AACAAGTACT | |
| | 961 | TCCACTCGCT TAAAAATAAC TAATAACAAT AATCCCTAAA | |
| SEQ ID | 1 | CTTTGTGATA ATCGTGGCAA TACTGATTAA TTTTGCTAAA CGTTTTTTCG AATATTTTTT | PScFIT3-813 |
| NO: 11 | 61 | TACTTTTCTT CCTACTGTCG AGAACATCCT TCGCGCAATG TAACCAAGAC CCTAGTGCTG | |
| | 121 | GTTCATAAGT GCAAAGAGTA GCGTGCCGAC CCTTCACATC CGTGTCAAAT TTGTGTGATT | |
| | 181 | CCAATTCTTT AGCACAAGCA TCAACTGCTA TCTGGTCCCA TTGCGTTCTT TTCTTAGTTG | |
| | 241 | ATGCCGGTTT TGCTAAAGAA CCTGGTGCCA AATATACCAA CAGCAGCACT AATCTAGCGA | |
| | 301 | AAAGCATTCG AAATCTGGGT GAATTAAATT AACTGCGACA AAATTGGTGA TGTGACATAC | |
| | 361 | TGAAAACGCT ACATTGCACA TTTGCCAAGT AAAAAATTGA CGCTCCTTTT TTAATGATCG | |
| | 421 | CTAATGTAAT GAAAAGTAAA AATCCAATTA AACGAGGAAG CGGTAAAATA TGATTGCTGT | |
| | 481 | GTAGTAATGC CACAGTCTTC TTAGTCCTGC CTATGTCCTA GATTTTTTTT AGCACCCTGT | |
| | 541 | TTGTTCCGCA CCCCGCTATC CCATATTTTT TGATGTATGT GAAGGAAAGC AATCTAAGGG | |
| | 601 | CTCAAGAGCC GAGTTCCGCC ATTCTAGGAA AGGGTGCAAA AAAATGGGGT TCAATTATCG | |
| | 661 | TTCGAGATTT GTTATTTTTT TTTGTAAAAG TTGATTTATG GGGTATTTAA GCCAAGAACT | |
| | 721 | ATTTGAAGTA TTAGGAGGTA ATATGAATGT TCGAACAGAT ACAAACAAGT ACTTCCACTC | |
| | 781 | GCTTAAAAAT AACTAATAAC AATAATCCCT AAA | |
| SEQ ID | 1 | CAAATTTGTG TGATTCCAAT TCTTTAGCAC AAGCATCAAT TGCTATCTGG TCCCATTGCG | PScFIT3-650 |
| NO: 12 | 61 | TTCTTTTCTT AGTTGATGCT GGTTTTGCTA AAGAACCTGG TGCCAAATAC ACCAACAGCA | |
| | 121 | GCACTAATCT AGCGAAAAGC ATTCGAAATC TGGGTGAATT AAATTAACTG CGACAAAATT | |
| | 181 | GGTGATGTGA CATACCGAAA ACGCTACATT ACACATTTGC CAAGTAAAAA ATTGACGCTC | |
| | 241 | CTTTTTTAAT GATCGCTAAT GTAATGAAAA GTAAAAATCC AATTAAACGA GGAAGCGGTA | |
| | 301 | AAATATGATT GCTGTGTAGT AATGCCACAG TCTTCTTAGT CCTACCTATG TCCTAGATTT | |
| | 361 | TTTTTTGCAC CCTGTTTGTT CCGCACCCCG CTATCCCATA TTTTTTGATG TATGTGAAGG | |
| | 421 | AAAGCAATCT AAGGGCTCAA GAGCCGAGTT CCGCCATTCT AGGAAAGGGT GCAAAAAAAT | |
| | 481 | GGGGTTCAAT TATCGTTCGA GATTTGTTAT TTTTTTTTTT GTAAAAGTTG ATTTATGGGG | |
| | 541 | TATTTAAGCC AAGAACTATT TGAAGTATTA GGAGGTAATA TGAATGTTCG AACAGATACA | |
| | 601 | AACAAGTACT TCCACTCGCT TAAAAATAAC TAATAACAAT AATCCCTAAA | |
| SEQ ID | 1 | AAAACGCTAC ATTACACATT TGCCAAGTAA AAAATTGACG CTCCTTTTTT AATGATCGCT | PScFIT3-453 |
| NO: 13 | 61 | AATGTAATGA AAAGTAAAAA TCCAATTAAA CGAGGAAGCG GTAAAATATG ATTGCTGTGT | |
| | 121 | AGTAATGCCA CAGTCTTCTT AGTCCTACCT ATGTCCTAGA TTTTTTTTTG CACCCTGTTT | |
| | 181 | GTTCCGCACC CCGCTATCCC ATATTTTTTG ATGTATGTGA AGGAAAGCAA TCTAAGGGCT | |
| | 241 | CAAGAGCCGA GTTCCGCCAT TCTAGGAAAG GGTGCAAAAA AATGGGGTTC AATTATCGTT | |
| | 301 | CGAGATTTGT TATTTTTTTT TTTGTAAAAG TTGATTTATG GGGTATTTAA GCCAAGAACT | |
| | 361 | ATTTGAAGTA TTAGGAGGTA ATATGAATGT TCGAACAGAT ACAAACAAGT ACTTCCACTC | |
| | 421 | GCTTAAAAAT AACTAATAAC AATAATCCCT AAA | |
| SEQ ID | 1 | TGTGTAGTAA TGCCACAGTC TTCTTAGTCC TACCTATGTC CTAGATTTTT TTTTGCACCC | PScFIT3-338 |
| NO: 14 | 61 | TGTTTGTTCC GCACCCCGCT ATCCCATATT TTTTGATGTA TGTGAAGGAA AGCAATCTAA | |
| | 121 | GGGCTCAAGA GCCGAGTTCC GCCATTCTAG GAAAGGGTGC AAAAAAATGG GGTTCAATTA | |
| | 181 | TCGTTCGAGA TTTGTTATTT TTTTTTTTGT AAAAGTTGAT TTATGGGGTA TTTAAGCCAA | |
| | 241 | GAACTATTTG AAGTATTAGG AGGTAATATG AATGTTCGAA CAGATACAAA CAAGTACTTC | |
| | 301 | CACTCGCTTA AAAATAACTA ATAACAATAA TCCCTAAA | |
| SEQ ID | 1 | CATATTTTTT GATGTATGTG AAGGAAAGCA ATCTAAGGGC TCAAGAGCCG AGTTCCGCCA | PScFIT3-254 |
| NO: 15 | 61 | TTCTAGGAAA GGGTGCAAAA AAATGGGGTT CAATTATCGT TCGAGATTTG TTATTTTTTT | |
| | 121 | TTTTGTAAAA GTTGATTTAT GGGGTATTTA AGCCAAGAAC TATTTGAAGT ATTAGGAGGT | |
| | 181 | AATATGAATG TTCGAACAGA TACAAACAAG TACTTCCACT CGCTTAAAAA TAACTAATAA | |
| | 241 | CAATAATCCC TAAA | |
| SEQ ID | 1 | TGGCATCAAA TTTGGGAAAA TTTAGCTTAC TCAGACACCA ACACATTTTT TCATGAGTAT | PScHXT6- |
| NO: 16 | 61 | TGTTGGAAAA TCTTTCCATG CTTATCAGCC GGAATACGGA ACAACGGCTG CTGTGATGCC | 1000 |
| | 121 | GCAAAGGGGC AAGGAAAAAA TAAGCAACAC ACACCGAAAC AGGAGGAAGA AATTCCGAAC | SGD ID |
| | 181 | ACAAGCCTAA GAAGACACCG CAGTTGTTGC AACATGTTCA CATGTTCATC CCCCTGCTAC | YDR34C |
| | 241 | TACTTGGAAA TTAATGTAGG GGTAATTGGA GTGCGCCGGC CCGTGCTTTC AGAGGAACAA | |
| | 301 | AGGAAGAATT GTTAAAAAAA GGCAGTGACA ACGAGCACGG GTGCACCATA TGGTGTGATA | |
| | 361 | AGCAACCTAG GACAAAAAGA AGTGCGCAGT TCCTGTGTGC GGCGCCAAGA CAAATGTTTC | |
| | 421 | ATATGCTTCT CCAAGGGGCT ACGCTACCTA GCCTCACCAC CCGATCTCTT TTTTTCCTGA | |
| | 481 | ACGCAGAGGG GACCGTACGA AAAAAAATGT TTTTTAGGCA ACGGAGATTC GTTTTATCCA | |
| | 541 | CGTTTACCCC ACAAAAAGTG CAGGTACATT GTGGGGCCCC GGCATCGAAA ACCAGTTTTT | |
| | 601 | TTCCTTTAAA CGCTGGAAAA AAAGGAGAAA TTATTGGAAC TTTGCAGAGA ATAGTCCGTA | |
| | 661 | GGCAAATTGA AAATGTTCCT TAAAAAATTT CGTTTCTTAC TCATTGAGAT TATTCAGATG | |
| | 721 | CCCTCCGTGC CTTCATTGAA AAAAATCCAA GAGATGTCTC GGATCTGTAT GCAGATTTTG | |
| | 781 | GCTTGCAGAC AATGGAGAGC AAATGGGTAT ACAATATAGA AAGCACAGAA ACATATAAAA | |
| | 841 | AGAGCTCGAG AAAAGACATA TGGTTTGTAA CTATCTTCTT CTTTTTTCCA ATTTTTCTGT | |
| | 901 | TTTAATAATA AAAAAACAAG AACAAACAAG CTCAACTTGT CTTTTCTAAG AACAAAGAAT | |
| | 961 | AAACACAAAA ACAAAAAGTT TTTTTAATTT TAATCAAAAA | |
| SEQ ID | 1 | CGCAAAGGGG CAAGGAAAAA ATAAGCAACA CACACCGAAA CAGGAGGAAG AAATTCCGAA | PScHXT6-881 |
| NO: 17 | 61 | CACAAGCCTA AGAAGACACC GCAGTTGTTG CAACATGTTC ACATGTTCAT CCCCCTGCTA | |
| | 121 | CTACTTGGAA ATTAATGTAG GGGTAATTGG AGTGCGCCGG CCCGTGCTTT CAGAGGAACA | |
| | 181 | AAGGAAGAAT TGTTAAAAAA AGGCAGTGAC AACGAGCACG GGTGCACCAT ATGGTGTGAT | |
| | 241 | AAGCAACCTA GGACAAAAAG AAGTGCGCAG TTCCTGTGTG CGGCGCCAAG ACAAATGTTT | |
| | 301 | CATATGCTTC TCCAAGGGGC TACGCTACCT AGCCTCACCA CCCGATCTCT TTTTTTCCTG | |
| | 361 | AACGCAGAGG GGACCGTACG AAAAAAAATG TTTTTTAGGC AACGGAGATT CGTTTTATCC | |
| | 421 | ACGTTTACCC CACAAAAAGT GCAGGTACAT TGTGGGGCCC CGGCATCGAA AACCAGTTTT | |
| | 481 | TTTCCTTTAA ACGCTGGAAA AAAAGGAGAA ATTATTGGAA CTTTGCAGAG AATAGTCCGT | |
| | 541 | AGGCAAATTG AAAATGTTCC TTAAAAAATT TCGTTTCTTA CTCATTGAGA TTATTCAGAT | |
| | 601 | GCCCTCCGTG CCTTCATTGA AAAAAATCCA AGAGATGTCT CGGATCTGTA TGCAGATTTT | |
| | 661 | GGCTTGCAGA CAATGGAGAG CAAATGGGTA TACAATATAG AAAGCACAGA AACATATAAA | |
| | 721 | AAGAGCTCGA GAAAAGACAT ATGGTTTGTA ACTATCTTCT TCTTTTTTCC AATTTTTCTG | |
| | 781 | TTTTAATAAT AAAAAAACAA GAACAAACAA GCTCAACTTG TCTTTTCTAA GAACAAAGAA | |
| | 841 | TAAACACAAA AACAAAAAGT TTTTTTAATT TTAATCAAAA A | |
| SEQ ID | 1 | TTTTTTTCCT GAACGCAGAG GGGACCGTAC GAAAAAAAAT GTTTTTTAGG CAACGGAGAT | PScHXT6-532 |
| NO: 18 | 61 | TCGTTTTATC CACGTTTACC CCACAAAAAG TGCAGGTACA TTGTGGGGCC CCGGCATCGA | |
| | 121 | AAACCAGTTT TTTTCCTTTA AACGCTGGAA AAAAAGGAGA AATTATTGGA ACTTTGCAGA | |
| | 181 | GAATAGTCCG TAGGCAAATT GAAAATGTTC CTTAAAAAAT TTCGTTTCTT ACTCATTGAG | |
| | 241 | ATTATTCAGA TGCCCTCCGT GCCTTCATTG AAAAAAATCC AAGAGATGTC TCGGATCTGT | |
| | 301 | ATGCAGATTT TGGCTTGCAG ACAATGGAGA GCAAATGGGT ATACAATATA GAAAGCACAG | |
| | 361 | AAACATATAA AAAGAGCTCG AGAAAAGACA TATGGTTTGT AACTATCTTC TTCTTTTTTC | |
| | 421 | CAATTTTTCT GTTTTAATAA TAAAAAAACA AGAACAAACA AGCTCAACTT GTCTTTTCTA | |
| | 481 | AGAACAAAGA ATAAACACAA AAACAAAAAG TTTTTTTAAT TTTAATCAAA AA | |
| SEQ ID | 1 | TCCGTAGGCA AATTGAAAAT GTTCCTTAAA AAATTTCGTT TCTTACTCAT TGAGATTATT | PScHXT6-346 |
| NO: 19 | 61 | CAGATGCCCT CCGTGCCTTC ATTGAAAAAA ATCCAAGAGA TGTCTCGGAT CTGTATGCAG | |
| | 121 | ATTTTGGCTT GCAGACAATG GAGAGCAAAT GGGTATACAA TATAGAAAGC ACAGAAACAT | |
| | 181 | ATAAAAAGAG CTCGAGAAAA GACATATGGT TTGTAACTAT CTTCTTCTTT TTTCCAATTT | |
| | 241 | TTCTGTTTTA ATAATAAAAA AACAAGAACA AACAAGCTCA ACTTGTCTTT TCTAAGAACA | |
| | 301 | AAGAATAAAC ACAAAAACAA AAAGTTTTTT TAATTTTAAT CAAAAA | |
| SEQ ID | 1 | AATGGAGAGC AAATGGGTAT ACAATATAGA AAGCACAGAA ACATATAAAA AGAGCTCGAG | PScHXT6-210 |
| NO: 20 | 61 | AAAAGACATA TGGTTTGTAA CTATCTTCTT CTTTTTTCCA ATTTTTCTGT TTTAATAATA | |
| | 121 | AAAAAACAAG AACAAACAAG CTCAACTTGT CTTTTCTAAG AACAAAGAAT AAACACAAAA | |
| | 181 | ACAAAAAGTT TTTTTAATTT TAATCAAAAA | |
| SEQ ID | 1 | ACAACACAAT GGTTAGTAGC AACCTGAATT CGGTCATTGA TGCATGCATG TGCCGTGAAG | PScENO1-726 |
| NO: 21 | 61 | CGGGACAACC AGAAAAGTCG TCTATAAATG CCGGCACGTG CGATCATCGT GGCGGGGTTT | SGD ID |
| | 121 | TAAGAGTGCA TATCACAAAT TGTCGCATTA CCGCGGAACC GCCAGATATT CATTACTTGA | YHR174W |
| | 181 | CGCAAAAGCG TTTGAAATAA TGACGAAAAA GAAGGAAGAA AAAAAAAGAA AAATACCGCT | |
| | 241 | TCTAGGCGGG TTATCTGCTA ATCCGAGCTT CCACTAGGAT AGCACCCAAA CACCTGCATA | |
| | 301 | TTTGGACGAC CTTTACTTAC ACCACCAAAA ACCACTTTCG CCTCTCCCGC CCCTGATAAC | |
| | 361 | GTCCACTAAT TGAGCGATTA CCTGAGCGGT CCTCTTTTGT TTGCAGCATG AGACTTGCAT | |
| | 421 | ACTGCAAATC GTAAGTAGCA ACGTCTCAAG GTCAAAACTG TATGGAAATC TTGTCACCTC | |
| | 481 | ACCTAATTCT AGCTAGCCTA CCCTGCAAGT CAAGAGGTCT CCGTGATTCC TAGCCACCTC | |
| | 541 | AAGGTATGCC TCTCCCCGGA AACTGTGGCC TTTTCTGGCA CACATGATCT CCACGATTTC | |
| | 601 | AACATATAAA TAGCTTTTGA TAATGGCAAT ATTAATCAAA TTTATTTTAC TTCTTTCTTG | |
| | 661 | TAACATCTCT CTTGTAATTC CTTATTCCTT CTAGCTATTT TTCATAAAAA ACCAAGCAAC | |
| | 721 | TGCTTA | |
| SEQ ID | 1 | ATGTTGACTA AAGCTACAAA AGAGCAGAAA TCATTGGTGA AAAATAGGGG TGCAGAACTT | Bacillus |
| NO: 22 | 61 | GTTGTGGACT GTTTGGTAGA ACAGGGCGTA ACACATGTTT TTGGTATCCC AGGTGCAAAA | subtilis |
| | 121 | ATCGACGCCG TGTTTGATGC ATTACAAGAC AAGGGTCCAG AAATTATTGT TGCTAGACAT | ilvB, |
| | 181 | GAGCAAAATG CCGCATTTAT GGCGCAAGCT GTAGGTAGGC TTACAGGTAA ACCTGGTGTT | Bs-ALS |
| | 241 | GTCCTAGTTA CGTCTGGCCC AGGAGCCTCC AATTTAGCAA CTGGTCTATT GACAGCTAAT | Acetolactate |
| | 301 | ACTGAGGGAG ATCCTGTAGT TGCGTTAGCC GGTAATGTAA TTAGAGCTGA TAGGCTTAAG | synthase |
| | 361 | AGAACTCACC AGTCTCTAGA CAACGCTGCT TTATTCCAAC CGATCACCAA GTACTCAGTA | |
| | 421 | GAGGTACAAG ACGTAAAGAA TATACCTGAA GCTGTGACAA ACGCATTTCG TATAGCTTCT | |
| | 481 | GCTGGTCAGG CTGGTGCCGC GTTTGTTTCT TTTCCTCAAG ACGTTGTCAA TGAAGTGACC | |
| | 541 | AATACTAAAA ACGTTAGAGC GGTTGCAGCC CCTAAACTAG GTCCAGCCGC AGACGACGCA | |
| | 601 | ATTAGCGCTG CAATTGCTAA AATTCAGACG GCGAAACTAC CAGTAGTCCT TGTCGGTATG | |
| | 661 | AAGGGCGGAA GACCAGAAGC AATAAAAGCT GTTCGTAAGT TATTGAAGAA AGTCCAATTA | |
| | 721 | CCTTTCGTTG AGACTTACCA AGCAGCAGGT ACTTTATCTA GAGATTTAGA GGATCAGTAT | |
| | 781 | TTTGGAAGGA TAGGTCTATT TAGAAACCAA CCAGGAGATT TACTATTAGA ACAAGCTGAT | |
| | 841 | GTTGTACTTA CTATCGGTTA TGATCCTATA GAGTATGACC CAAAGTTTTG GAACATAAAT | |
| | 901 | GGGGATAGAA CAATTATACA TCTAGACGAG ATAATCGCCG ACATCGATCA CGCTTATCAA | |
| | 961 | CCAGATTTAG AACTAATCGG AGATATCCCG TCAACAATCA ATCATATTGA ACATGATGCT | |
| | 1021 | GTAAAGGTTG AGTTCGCTGA ACGTGAGCAG AAAATCTTAT CTGATCTAAA GCAATATATG | |
| | 1081 | CATGAGGGTG AACAAGTTCC AGCAGACTGG AAATCTGACC GTGCACATCC TTTGGAAATC | |
| | 1141 | GTTAAGGAAC TAAGAAATGC GGTCGATGAT CATGTGACTG TTACATGTGA TATCGGTTCA | |
| | 1201 | CATGCAATTT GGATGTCACG TTATTTTAGG AGCTACGAAC CATTAACTTT AATGATATCT | |
| | 1261 | AACGGGATGC AAACTCTGGG GGTTGCACTT CCTTGGGCTA TTGGCGCTAG TTTAGTTAAG | |
| | 1321 | CCCGGTGAGA AGGTGGTATC GGTATCAGGT GATGGTGGCT TTCTGTTTTC GGCTATGGAA | |
| | 1381 | TTAGAAACTG CAGTCCGTTT AAAAGCTCCC ATTGTGCATA TTGTCTGGAA TGATTCTACT | |
| | 1441 | TACGACATGG TTGCTTTTCA ACAGTTGAAG AAATACAATA GAACTTCGGC TGTAGACTTT | |
| | 1501 | GGTAACATCG ATATTGTGAA ATATGCTGAG TCTTTTGGCG CAACAGGCCT GAGGGTGGAA | |
| | 1561 | AGTCCAGATC AGTTAGCTGA TGTGTTGAGA CAAGGGATGA ATGCCGAGGG ACCGGTAATC | |
| | 1621 | ATAGATGTGC CAGTTGACTA CTCAGACAAT ATTAATTTGG CTTCTGATAA ACTTCCTAAA | |
| | 1681 | GAGTTTGGCG AGCTAATGAA GACCAAAGCC TTATAA | |
| SEQ ID | 1 | ATGTCCGTCA AGACAAAAGA AAAGGAAATG GCAGTTACAA TTTTATACGA ACAAGACGTT | Slackia |
| NO: 23 | 61 | GATCCAAAGG TTATCCAAGG TTTGAAGGTT GGTATCATAG GTTACGGTTC TCAAGGTCAC | Exigua |
| | 121 | GCTCATGCAT TGAACTTAAT GGATTCAGGT GTTGATGTCA GAGTTGGTTT GAGAGAAGGT | ilvC, |
| | 181 | GACTCTGATT GGAAGACAGC CGAAGAAGCT GGTTTAAAGG TTACAGATAT TGACACTGCC | Sel-KARI |
| | 241 | GCAGAAGAAG CAGATGTTAT TATGGTCTTA ATTCCTGATG AAGTTCAACC AAAGGTTTAC | Ketol-acid |
| | 301 | CAAGAACATA TTGCAGCTCA TTTGAAGGCT GGTAATACCT TGGCTTTTGC ACACGGTTTC | reducto- |
| | 361 | AATATCCATT ACGGTTATAT CGTTCCTCCA GAAGACGTTA ATGTCATCAT GTGTGCTCCT | isomerase |
| | 421 | AAAGGTCCAG GTCACATCGT TAGAAGACAA TTCACCGAAG GTTCAGGTGT CCCTGATTTG | |
| | 481 | GCATGTGTTC AACAAGATGC AACTGGTAAT GCATGGGATA TTGTTTTATC CTATTGTTGG | |
| | 541 | GGTGTTGGTG GTGCAAGATC AGGTATCATA AAAGCTACAT TCGCAGAAGA AACAGAAGAA | |
| | 601 | GACTTGTTTG GTGAACAAGC AGTTTTGTGT GGTGGTTTAG TTGAATTGGT CAAAGCTGGT | |
| | 661 | TTTGAAACCT TGACTGAAGC TGGTTATCCA CCTGAATTGG CCTACTTTGA ATGTTATCAC | |
| | 721 | GAAATGAAGA TGATCGTTGA CTTGATGTAT GAATCTGGTA TTCATTTTAT GAACTACTCA | |
| | 781 | ATCTCTAACA CTGCAGAATA CGGTGAATAC TATGCCGGTC CTAAAGTCAT CAATGAACAA | |
| | 841 | AGTAGAGAAG CAATGAAAGA AATCTTGGCT AGAATCCAAG ACGGTTCATT TGCTCAAGAA | |
| | 901 | TTTGTCGATG ACTGCAATAA TGGTCATAAG AGATTGTTAG AACAAAGAGA AGCTATTAAC | |
| | 961 | ACTCATCCAA TCGAAACTAC AGGTGCTAGA ATCAGATCAA TGTTTTCTTG GATAAAGAAG | |
| | 1021 | GAAGATTTGG AATGA | |
| SEQ ID | 1 | ATGGAATTTA AATATAATGG TAAAACAGAA GCCATCGAAC TTAACAAATA CTCAAAAACA | Lactococcus |
| NO: 24 | 61 | CTAACCCAAG ATCCCACTCA ACCAGCAACT CAAGCGATGT ACTACGGTAT CGGTTTTAAA | Lactis |
| | 121 | GATGAAGACT TTAAAAAAGC CCAAGTCGGC ATTGTCAGTA TGGACTGGGA CGGAAATCCC | ilvD, |
| | 181 | TGTAATATGC ATCTTGGAAC ACTTGGTGGG AAAATCAAAG CCTCTGTCAA TCAAACAGAA | Ll- |
| | 241 | GGATTAATTG GTTTACAATT TCATACGATC GGCGTATCGG ATGGGATTGC AAACGGTAAA | DHAD |
| | 301 | CTTGGAATGA GATATAGCTT GGTAAGCCGT GAGGTCATTG CTGACAGTAT TGAGACCAAC | Dihydroxy- |
| | 361 | GCTGGCGCAG AATACTACGA TGCGATTGTT TCTGTACCAG GCTGTGACAA AAATATGCCA | acid |
| | 421 | GGCTCGATTA TTGGAATGGC GAGACTCAAT CGACCTTCAA TCATGGTTTA CGGTGGAACA | dehydratase |
| | 481 | ATAGAACACG GTGAATACAA AGGTGAAAAA CTAAACATTG TTTCAGCTTT TGAAGCATTA | |
| | 541 | GGGCAAAAAA TTACAGGTAA TATCTCCGAA GAAGATTATC ATGGTTGTAT CAAAAATGCA | |
| | 601 | ATTCCAGGAG CTGGGGCTTG TGGTGGAATG TATACAGCAA ATACATTAGC GTCAGCAATT | |
| | 661 | GAAACGCTTG GAATGAGTTT GCCATATTCT GCATCAAATC CTGCGGTCAG TCCAGAAAAA | |
| | 721 | CAAGAAGAAT GTGATGAAAT CGGCGCAGCA ATCAAAAATT TGCTTGAAAA GGATATTAAG | |
| | 781 | CCAAGTGATA TTATGACCAA AGAAGCGTTT GAAAACGCAA TTACGATTGT CATGGTGCTT | |
| | 841 | GGTGGCTCGA CTAATGCGGT ATTACATATC ATTGCAATGG CAAATGCCAT CGGTGTTGAA | |
| | 901 | ATTACGCAAG ACGACTTCCA AAGAATTTCA GATGTAACAC CTGTTCTTGG TGATTTTAAG | |
| | 961 | CCAAGCGGAA AGTACATGAT GGAAGACTTA CACAAAATTG GTGGTGTGCC TGCCGTTCTC | |
| | 1021 | AAATATTTGC TCAAAGAAGG TAAACTTCAC GGAGACTGTT TAACGGTTAC AGGTAAAACT | |
| | 1081 | TTAGCTGAAA ATGTTGCCAA TGCGCTTGAT TTGGACTTTG AAAATCAAGA CATCATGCAT | |
| | 1141 | TCGCTTTCTG ATCCAATCAA AGCAACAGGA CATTTACAAA TTTTATATGG CAACCTCGCT | |
| | 1201 | GAAGGTGGTT CTGTCGCAAA AATTTCTGGT AAAGAAGGCG AATTTTTCAA AGGAACGGCG | |
| | 1261 | CGAGTCTTTG ATGGCGAGCA ACATTTCATT GATGGGATTG AGTCTGGAAG GTTGCACGCA | |
| | 1321 | GGCGATGTTG CTGTCATACG TAATATTGGT CCTGTCGGTG GTCCAGGAAT GCCTGAAATG | |
| | 1381 | CTTAAACCAA CCTCTGCTTT GATTGGTGCT GGACTTGGCA AATCTTGTGC GCTGATTACA | |
| | 1441 | GATGGAAGAT TTTCAGGTGG GACACACGGA TTTGTTGTAG GTCATATTGT TCCTGAAGCT | |
| | 1501 | GTTGAAGGTG GATTGATCGG TTTAGTTGAA GATGAGGACA TCATCGAAAT TGATGCTACT | |
| | 1561 | AACAATACGA TAACATTGAA AGTTGATGAT GAGGAAATCG CGCGTCGTCG TGCAGCATAC | |
| | 1621 | GTGCGTCCGT TACCCAAAGC AACACGTGGT GTACTCGCAA AATTTGCAAA ACTGACACGA | |
| | 1681 | CCTGCTAGTG AGGGCTGTGT GACAGATTTA TGA | |
| SEQ ID | 1 | ATGTATACAG TGGGTGATTA CTTGCTAGAC CGTTTACATG AATTAGGCAT AGAAGAGATT | Lactococcus |
| NO: 25 | 61 | TTTGGAGTAC CAGGTGATTA CAATTTGCAA TTCTTGGATC AGATTATCTC AAGAAAGGAT | lactis |
| | 121 | ATGAAATGGG TCGGGAATGC GAACGAGTTA AACGCTTCCT ATATGGCAGA TGGTTATGCG | ilvE, Ll |
| | 181 | AGAACCAAAA AGGCCGCTGC TTTTCTTACA ACTTTCGGTG TAGGTGAACT TTCAGCCGTT | KIVD |
| | 241 | AATGGATTAG CCGGATCTTA CGCTGAAAAC TTACCAGTCG TTGAAATTGT TGGTTCTCCT | Keto- |
| | 301 | ACTTCTAAGG TACAAAACGA GGGAAAGTTT GTTCACCATA CTTTAGCGGA TGGTGATTTC | isovalerate |
| | 361 | AAACATTTCA TGAAGATGCA TGAACCTGTC ACAGCAGCGA GAACACTTTT GACCGCAGAG | dehydro- |
| | 421 | AACGCTACTG TTGAAATCGA TAGAGTATTA AGTGCTTTGT TAAAAGAGAG AAAGCCAGTG | genase |
| | 481 | TATATCAACT TGCCTGTTGA TGTCGCTGCC GCAAAAGCAG AAAAACCATC TTTACCATTG | |
| | 541 | AAAAAGGAAA ACTCAACCAG TAACACATCT GATCAAGAGA TTCTAAACAA AATCCAAGAG | |
| | 601 | TCATTGAAAA ACGCCAAAAA GCCAATCGTT ATAACAGGCC ATGAAATCAT CTCCTTTGGT | |
| | 661 | TTAGAAAAGA CCGTTTCACA ATTCATCTCT AAGACAAAAT TGCCAATCAC TACTTTAAAC | |
| | 721 | TTTGGCAAAT CTTCTGTAGA TGAAGCTTTA CCATCTTTTC TAGGTATCTA CAATGGTAAA | |
| | 781 | CTTTCTGAAC CAAACCTAAA AGAGTTTGTC GAATCCGCTG ATTTCATACT TATGTTGGGT | |
| | 841 | GTTAAGCTAA CTGATAGTTC AACTGGTGCA TTCACTCACC ACTTGAACGA AAACAAGATG | |
| | 901 | ATATCATTGA ATATCGATGA GGGCAAGATA TTCAACGAAT CAATTCAAAA CTTCGATTTT | |
| | 961 | GAATCACTAA TTTCTTCTCT ACTTGATTTG TCAGAAATAG AATACAAAGG AAAATACATC | |
| | 1021 | GACAAAAAGC AAGAGGATTT CGTGCCATCA AACGCATTGT TGTCACAAGA TAGACTTTGG | |
| | 1081 | CAAGCTGTGG AAAACCTAAC TCAATCTAAC GAAACAATTG TGGCTGAGCA AGGCACATCA | |
| | 1141 | TTCTTCGGCG CATCTAGTAT TTTCTTGAAG CCAAAGTCCC ACTTCATCGG TCAACCTCTT | |
| | 1201 | TGGGGATCTA TTGGGTACAC CTTCCCTGCC GCATTGGGAT CACAGATCGC AGACAAGGAG | |
| | 1261 | TCCAGACATC TACTATTCAT TGGGGACGGA TCATTGCAAC TTACCGTTCA GGAATTAGGG | |
| | 1321 | TTGGCTATAA GAGAAAAGAT CAATCCTATT TGTTTTATCA TCAACAATGA TGGCTATACA | |
| | 1381 | GTAGAACGTG AGATTCACGG TCCAAATCAA AGTTACAATG ACATCCCAAT GTGGAATTAC | |
| | 1441 | TCTAAGTTGC CTGAATCTTT CGGTGCAACA GAAGAAAGAG TTGTTTCCAA AATAGTCAGA | |
| | 1501 | ACAGAAAATG AGTTTGTTTC TGTCATGAAG GAAGCTCAAG CCGATCCTAA TAGAATGTAC | |
| | 1561 | TGGATTGAAC TAATCTTGGC CAAGGAAGAT GCCCCAAAAG TATTGAAAAA GATGGGAAAA | |
| | 1621 | CTTTTCGCTG AACAGAATAA GTCCTGA | |
| SEQ ID | 1 | ATGAAAGCAG CAGTAGTAAG ACACAATCCA GATGGTTATG CGGACCTTGT TGAAAAGGAA | Lactococcus |
| NO: 26 | 61 | CTTCGAGCAA TCAAACCTAA TGAAGCTTTG CTTGACATGG AGTATTGTGG AGTCTGTCAT | lactis |
| | 121 | ACCGATTTGC ACGTAGCAGC AGGTGATTTT GGCAACAAAG CAGGGACTGT TCTTGGTCAT | ADH1, Ll |
| | 181 | GAAGGAATTG GAATTGTCAA AGAAATTGGA GCTGATGTAA GCTCGCTTCA AGTTGGTGAT | ADH |
| | 241 | CGGGTTTCAG TGGCTTGGTT CTTTGAAGGA TGTGGTCACT GTGAATACTG TGTATCTGGT | Alcohol |
| | 301 | AATGAAACTT TTTGTCGAGA AGTTAAAAGT GCAGGATATT CAGTTGATGG CGGAATGGCT | dehydro- |
| | 361 | GAAGAAGCAA TTGTTGTTGC CGATTATGCT GTCAAAGTTC CTGACGGACT TGACCCAATT | genase |
| | 421 | GAAGCTAGCT CAATTACTTG TGCTGGAGTA ACAACTTACA AAGCAATCAA AGTATCAGGA | |
| | 481 | GTAAAACCTG GTGATTGGCA AGTAATTTTT GGTGCTGGAG GACTTGGAAA TTTAGCAATT | |
| | 541 | CAATATGCTA AAAATGTTTT TGGAGCAAAA GTAATTGCTG TTGATATTAA TCAAGATAAA | |
| | 601 | TTAAATTTAG CTAAAAAAAT TGGAGCTGAT GTGACTATCA ATTCTGGTGA TGTAAATCCA | |
| | 661 | GTTGATGAAA TTAAAAAAAT AACTGGCGGC TTAGGGGTGC AAAGTGCAAT AGTTTGTGCT | |
| | 721 | GTTGCAAGGA TTGCTTTTGA ACAAGCGGTT GCTTCTTTGA AACCTATGGG CAAAATGGTT | |
| | 781 | GCTGTGGCAG TTCCCAATAC TGAGATGACT TTATCAGTTC CAACAGTTGT TTTTGACGGA | |
| | 841 | GTGGAGGTTG CAGGTTCACT TGTAGGAACA AGACTTGACT TGGCAGAAGC TTTTCAATTT | |
| | 901 | GGAGCAGAAG GTAAGGTAAA ACCAATTGTT GCGACACGCA AACTGGAAGA AATCAATGAT | |
| | 961 | ATTATTGATG AAATGAAGGC AGGAAAAATT GAAGGCCGAA TGGTCATTGA TTTTACTAAA | |
| | 1021 | TAA | |

TABLE 2

| SEQ ID | | | |
|---|---|---|---|
| NO: | Protein Sequence | | Description |
| SEQ ID | 1 | MLTKATKEQK SLVKNRGAEL VVDCLVEQGV THVFGIPGAK IDAVFDALQD KGPEIIVARH | Bacillus |
| NO: 27 | 61 | EQNAAFMAQA VGRLTGKPGV VLVTSGPGAS NLATGLLTAN TEGDPVVALA GNVIRADRLK | subtilis |
| | 121 | RTHQSLDNAA LFQPITKYSV EVQDVKNIPE AVTNAFRIAS AGQAGAAFVS FPQDVVNEVT | ilvB, Bs-ALS |
| | 181 | NTKNVRAVAA PKLGPAADDA ISAAIAKIQT AKLPVVLVGM KGGRPEAIKA VRKLLKKVQL | Acetolactate |
| | 241 | PFVETYQAAG TLSRDLEDQY FGRIGLFRNQ PGDLLLEQAD VVLTIGYDPI EYDPKFWNIN | synthase |
| | 301 | GDRTIIHLDE IIADIDHAYQ PDLELIGDIP STINHIEHDA VKVEFAEREQ KILSDLKQYM | |
| | 361 | HEGEQVPADW KSDRAHPLEI VKELRNAVDD HVTVTCDIGS HAIWMSRYFR SYEPLTLMIS | |
| | 421 | NGMQTLGVAL PWAIGASLVK PGEKVVSVSG DGGFLFSAME LETAVRLKAP IVHIVWNDST | |
| | 481 | YDMVAFQQLK KYNRTSAVDF GNIDIVKYAE SFGATGLRVE SPDQLADVLR QGMNAEGPVI | |
| | 541 | IDVPVDYSDN INLASDKLPK EFGELMKTKA L- | |
| SEQ ID | 1 | MSVKTKEKEM AVTILYEQDV DPKVIQGLKV GIIGYGSQGH AHALNLMDSG VDVRVGLREG | Slackia |
| NO: 28 | 61 | DSDWKTAEEA GLKVTDIDTA AEEADVIMVL IPDEVQPKVY QEHIAAHLKA GNTLAFAHGF | Exigua |
| | 121 | NIHYGYIVPP EDVNVIMCAP KGPGHIVRRQ FTEGSGVPDL ACVQQDATGN AWDIVLSYCW | ilvC, |
| | 181 | GVGGARSGII KATFAEETEE DLFGEQAVLC GGLVELVKAG FETLTEAGYP PELAYFECYH | Sel-KARI |
| | 241 | EMKMIVDLMY ESGIHFMNYS ISNTAEYGEY YAGPKVINEQ SREAMKEILA RIQDGSFAQE | Ketol-acid |
| | 301 | FVDDCNNGHK RLLEQREAIN THPIETTGAR IRSMFSWIKK EDLE- | reducto- |
| | | | isomerase |
| SEQ ID | 1 | MEFKYNGKTE AIELNKYSKT LTQDPTQPAT QAMYYGIGFK DEDFKKAQVG IVSMDWDGNP | Lactococcus |
| NO: 29 | 61 | CNMHLGTLGG KIKASVNQTE GLIGLQFHTI GVSDGIANGK LGMRYSLVSR EVIADSIETN | Lactis ilvD, |
| | 121 | AGAEYYDAIV SVPGCDKNMP GSIIGMARLN RPSIMVYGGT IEHGEYKGEK LNIVSAFEAL | Ll-DHAD |
| | 181 | GQKITGNISE EDYHGCIKNA IPGAGACGGM YTANTLASAI ETLGMSLPYS ASNPAVSPEK | Dihydroxy- |
| | 241 | QEECDEIGAA IKNLLEKDIK PSDIMTKEAF ENAITIVMVL GGSTNAVLHI IAMANAIGVE | acid |
| | 301 | ITQDDFQRIS DVTPVLGDFK PSGKYMMEDL HKIGGVPAVL KYLLKEGKLH GDCLTVTGKT | dehydratase |
| | 361 | LAENVANALD LDFENQDIMH SLSDPIKATG HLQILYGNLA EGGSVAKISG KEGEFFKGTA | |
| | 421 | RVFDGEQHFI DGIESGRLHA GDVAVIRNIG PVGGPGMPEM LKPTSALIGA GLGKSCALIT | |
| | 481 | DGRFSGGTHG FVVGHIVPEA VEGGLIGLVE DEDIIEIDAT NNTITLKVDD EEIARRRAAY | |
| | 541 | VRPLPKATRG VLAKFAKLTR PASEGCVTDL- | |
| SEQ ID | 1 | MYTVGDYLLD RLHELGIEEI FGVPGDYNLQ FLDQIISRKD MKWVGNANEL NASYMADGYA | Lactococcus |
| NO: 30 | 61 | RTKKAAAFLT TFGVGELSAV NGLAGSYAEN LPVVEIVGSP TSKVQNEGKF VHHTLADGDF | lactis ilvE, |
| | 121 | KHFMKMHEPV TAARTLLTAE NATVEIDRVL SALLKERKPV YINLPVDVAA AKAEKPSLPL | Ll |
| | 181 | KKENSTSNTS DQEILNKIQE SLKNAKKPIV ITGHEIISFG LEKTVSQFIS KTKLPITTLN | KIVD |
| | 241 | FGKSSVDEAL PSFLGIYNGK LSEPNLKEFV ESADFILMLG VKLTDSSTGA FTHHLNENKM | Keto- |
| | 301 | ISLNIDEGKI FNESIQNFDF ESLISSLLDL SEIEYKGKYI DKKQEDFVPS NALLSQDRLW | isovalerate |
| | 361 | QAVENLTQSN ETIVAEQGTS FFGASSIFLK PKSHFIGQPL WGSIGYTFPA ALGSQIADKE | dehydro- |
| | 421 | SRHLLFIGDG SLQLTVQELG LAIREKINPI CFIINNDGYT VEREIHGPNQ SYNDIPMWNY | genase |
| | 481 | SKLPESFGAT EERVVSKIVR TENEFVSVMK EAQADPNRMY WIELILAKED APKVLKKMGK | |
| | 541 | LFAEQNKS- | |
| SEQ ID | 1 | MKAAVVRHNP DGYADLVEKE LRAIKPNEAL LDMEYCGVCH TDLHVAAGDF GNKAGTVLGH | Lactococcus |
| NO: 31 | 61 | EGIGIVKEIG ADVSSLQVGD RVSVAWFFEG CGHCEYCVSG NETFCREVKS AGYSVDGGMA | lactis ADH1, |
| | 121 | EEAIVVADYA VKVPDGLDPI EASSITCAGV TTYKAIKVSG VKPGDWQVIF GAGGLGNLAI | Ll ADH |
| | 181 | QYAKNVFGAK VIAVDINQDK LNLAKKIGAD VTINSGDVNP VDEIKKITGG LGVQSAIVCA | Alcohol |
| | 241 | VARIAFEQAV ASLKPMGKMV AVAVPNTEMT LSVPTVVFDG VEVAGSLVGT RLDLAEAFQF | dehydro- |
| | 301 | GAEGKVKPIV ATRKLEEIND IIDEMKAGKI EGRMVIDFTK- | genase |

Recombinant microorganisms that produce alcohol are known in the art (e.g., Ohta et al., Appl. Environ. Microbiol. 57:893-900 (1991); Underwood et al., Appl. Environ. Microbiol. 68:1071-81 (2002); Shen and Liao, Metab. Eng. 10:312-20 (2008); Hahnai et al., Appl. Envrion. Microbiol. 73:7814-8 (2007); U.S. Pat. Nos. 5,514,583; 5,712,133; International Pub. No. WO 1995/028476; Feldmann et al., Appl. Microbiol. Biotechnol. 38:354-61 (1992); Zhang et al., Science 267:240-3 (1995); U.S. Pat. Pub. No. 2007/0031918A1; U.S. Pat. Nos. 7,223,575; 7,741,119; U.S. Pat. Pub. No. 2009/0203099A1; U.S. Pat. Pub. No. 2009/0246846A1; and International Pub. No. WO 2010/075241), which are herein incorporated by reference in their entirety.

For example, the metabolic pathways of microorganisms may be genetically modified to produce the alcohol butanol. The metabolite butanol may be produced by a recombinant microorganism metabolically engineered to express or over-express a metabolic pathway that converts pyruvate to butanol. The metabolic pathway may also be modified to reduce or eliminate undesired metabolites or co-products, thereby improving the yield of the alcohol. The production of butanol by a recombinant microorganism is disclosed in, for example, U.S. Pat. Nos. 7,851,188; 7,993,889; 8,178,328; 8,206,970; and 9,790,521; and U.S. Pat. App. Pub. Nos. 2007/0292927; 2008/0182308; 2008/0274525; 2009/0305363; 2009/0305370; 2011/0250610; 2011/0313206; 2011/0111472; and 2012/0258873, which are herein incorporated by reference in their entirety. Recombinant microorganisms that produce butanol at higher yields are disclosed in U.S. Pat. No. 8,455,239 and International Pub. No. WO 2010/05125, which are herein incorporated by reference in their entirety.

In some embodiments, the recombinant microorganism comprises a butanol biosynthetic pathway (e.g., an enzyme pathway) for producing butanol and/or isomers thereof, such as 1-butanol, 2-butanol, or isobutanol. Components of such pathways may include substrates, cofactors, byproducts, intermediates, end products, and/or enzymes in the pathways. In certain embodiments, the recombinant microorganism comprises a 2-butanone biosynthetic pathway (e.g., an enzyme pathway) to produce 2-butanone.

In some embodiments, the biosynthetic pathway converts pyruvate to a fermentative product. In some embodiments, the biosynthetic pathway converts pyruvate as well as amino acids to a fermentative product. In some embodiments, at least one, or at least two, or at least three, or at least four polypeptides catalyzing substrate-to-product conversions of a pathway are encoded by heterologous polynucleotides in the microorganism. In some embodiments, all polypeptides catalyzing substrate-to-product conversions of a pathway are encoded by heterologous polynucleotides in the microorganism.

Suitable microorganisms capable of producing alcohol (e.g., butanol) via a biosynthetic pathway include members of the genera Schizosaccharomyces, Kluveromyces, and Saccharomyces. In some embodiments, the recombinant microorganisms may be selected from the group consisting of Kluyveromyces lactis, Kluyveromyces marxianus, Kluveromyces thermotolerans, and Saccharomyces cerevisiae.

In some embodiments, the genetically modified microorganism is a recombinant yeast. In some embodiments, the genetically modified microorganism is a crabtree-positive recombinant yeast. Suitable types of yeast include Saccharomyces, Zygosaccharomyces, Schizosaccharomyces, Dekkera, Torulopsis, Brettanomyces, and some species of Candida. Species of crabtree-positive yeast include, but are not limited to, Saccharomyces cerevisiae, Saccharomyces kluyveri, Schizosaccharomyces pombe, Saccharomyces bayanus, Saccharomyces mikitae, Saccharomyces paradoxus, Saccharomyces uvarum, Saccharomyces castelli, Zygosaccharomyces rouxii, Zygosaccharomyces bailli, and Candida glabrata.

In some embodiments, the host cell is Saccharomyces cerevisiae. Saccharomyces cerevisiae is known in the art and available from a variety of sources including, but not limited to, American Type Culture Collection (Rockville, Md.), Centraalbureau voor Schimmelcultures (CBS) Fungal Biodiversity Centre, LeSaffre, Gert Strand AB, Ferm Solutions, North American Bioproducts, Martrex, and Lallemand. S. cerevisiae include, but are not limited to, BY4741, CEN.PK 113-7D, Ethanol Red® yeast, Ferm Pro™ yeast, Bio-Ferm® XR yeast, Gert Strand Prestige Batch Turbo alcohol yeast, Gert Strand Pot Distillers yeast, Gert Strand Distillers Turbo yeast, FerMax™ Green yeast, FerMax™ Gold yeast, Thermosacc® yeast, BG-1, PE-2, CAT-1, CBS7959, CBS7960, and CBS7961. A recombinant host cell comprising an “engineered alcohol production pathway” (such as an engineered butanol or isobutanol production pathway) refers to a host cell containing a modified pathway that produces alcohol in a manner different than that normally present in the host cell. Such differences include production of an alcohol not typically produced by the host cell or increased/more efficient production.

Biosynthetic pathways for the production of isobutanol that may be used include those described in U.S. Pat. Nos. 7,851,188; 7,993,388; and International Pub. No. WO 2007/050671, which are incorporated herein by reference in their entirety. In some embodiments, the isobutanol biosynthetic pathway may comprise the following substrate-to-product conversions:
a) pyruvate to acetolactate, which may be catalyzed by, for example, acetolactate synthase;
b) the acetolactate from step a) to 2,3-dihydroxyisovalerate, which may be catalyzed by, for example, ketol-acid reductoisomerase;
c) the 2,3-dihydroxyisovalerate from step b) to α-ketoisovalerate, which may be catalyzed by, for example, dihydroxy acid dehydratase;
d) the α-ketoisovalerate from step c) to isobutyraldehyde, which may be catalyzed by, for example, keto-isovalerate dehydrogenase; and,
e) the isobutyraldehyde from step d) to isobutanol, which may be catalyzed by, for example, a branched-chain alcohol dehydrogenase.

In some embodiments, the isobutanol biosynthetic pathway may comprise the following substrate-to-product conversions:
a) pyruvate to acetolactate, which may be catalyzed by, for example, acetolactate synthase;
b) the acetolactate from step a) to 2,3-dihydroxyisovalerate, which may be catalyzed by, for example, acetohydroxy acid reductoisomerase;
c) the 2,3-dihydroxyisovalerate from step b) to α-ketoisovalerate, which may be catalyzed by, for example, acetohydroxy acid dehydratase;
d) the α-ketoisovalerate from step c) to isobutyryl-CoA, which may be catalyzed by, for example, branched-chain keto acid dehydrogenase;
e) the isobutyryl-CoA from step d) to isobutyraldehyde, which may be catalyzed by, for example, acylating aldehyde dehydrogenase; and,
f) the isobutyraldehyde from step e) to isobutanol, which may be catalyzed by, for example, a branched-chain alcohol dehydrogenase.

The terms “acetohydroxyacid synthase,” “acetolactate synthase,” and “acetolactate synthetase” (abbreviated “ALS”) are used interchangeably herein to refer to an enzyme that catalyzes the conversion of pyruvate to acetolactate and CO₂. Example acetolactate synthases are known by the EC number 2.2.1.6 (Enzyme Nomenclature 1992, Academic Press, San Diego). These enzymes are available from a number of sources, including, but not limited to, Bacillus subtilis (GenBank Nos: CAB07802.1, Z99122, NCBI (National Center for Biotechnology Information) amino acid sequence, NCBI nucleotide sequence, respectively), CAB15618), Klebsiella pneumoniae (GenBank Nos: AAA25079, M73842), and Lactococcus lactis (GenBank Nos: AAA25161, L16975).

The term “ketol-acid reductoisomerase” (“KARI”), “acetohydroxy acid isomeroreductase,” and “acetohydroxy acid reductoisomerase” will be used interchangeably and refer to enzymes capable of catalyzing the reaction of (S)-acetolactate to 2,3-dihydroxyisovalerate. Example KARI enzymes may be classified as EC number EC 1.1.1.86 (Enzyme Nomenclature 1992, Academic Press, San Diego) and are available from a vast array of microorganisms, including, but not limited to, Escherichia coli (GenBank Nos: NP_418222, NC_000913), Saccharomyces cerevisiae (GenBank Nos: NP_013459, NC_001144), Methanococcus maripaludis (GenBank Nos: CAF30210, BX957220), Bacillus subtilis (GenBank Nos: CAB14789, Z99118), and Anaerostipes caccae. Ketol-acid reductoisomerase (KARI) enzymes are described in U.S. Pat. Nos. 7,910,342; 8,129,162; and 9,512,408; U.S. Pat. App. Pub. Nos. 2008/0261230; 2009/0163376; 2010/0197519; and International Pub. Nos. WO 2011/041415 and WO 2012/129555, which are incorporated herein by reference in their entirety. Examples of suitable KARIs include those from Lactococcus lactis, Vibrio cholera, Pseudomonas aeruginosa PAO1, and Pseudomonas fluorescens PF5 mutants. In some embodiments, the KARI uses NADH or NADPH.

The term “acetohydroxy acid dehydratase” and “dihydroxyacid dehydratase” (“DHAD”) refers to an enzyme that catalyzes the conversion of 2,3-dihydroxyisovalerate to α-ketoisovalerate. Example acetohydroxy acid dehydratases are known by the EC number 4.2.1.9. Such enzymes are available from a vast array of microorganisms, including, but not limited to, E. coli (GenBank Nos: YP_026248, NC000913), Saccharomyces cerevisiae (GenBank Nos: NP_012550, NC_001142), M. maripaludis (GenBank Nos: CAF29874, BX957219), B. subtilis (GenBank Nos: CAB14105, Z99115), L. lactis, and N. crassa. U.S. Pat. App. Pub. No. 2010/0081154; U.S. Pat. Nos. 7,851,188; and 8,241,878, which are incorporated herein by reference in their entireties, describe dihydroxyacid dehydratases (DHADs), including a DHAD from Streptococcus mutans and variants thereof.

The term “branched-chain α-keto acid decarboxylase,” “α-ketoacid decarboxylase,” “α-ketoisovalerate decarboxylase,” or “2-ketoisovalerate decarboxylase” (“KIVD”) refers to an enzyme that catalyzes the conversion of α-ketoisovalerate to isobutyraldehyde and CO₂. Example branched-chain α-keto acid decarboxylases are known by the EC number 4.1.1.72 and are available from a number of sources, including, but not limited to, Lactococcus lactis (GenBank Nos: AAS49166, AY548760; CAG34226, AJ746364), Salmonella typhimurium (GenBank Nos: NP_461346, NC_003197), Clostridium acetobutylicum (GenBank Nos: NP_149189, NC_001988), M. caseolyticus, and L. grayi.

The term “branched-chain alcohol dehydrogenase” (“ADH”) refers to an enzyme that catalyzes the conversion of isobutyraldehyde to isobutanol. Example branched-chain alcohol dehydrogenases are known by the EC number 1.1.1.265 but may also be classified under other alcohol dehydrogenases (specifically, EC 1.1.1.1 or 1.1.1.2). Alcohol dehydrogenases may be NADPH-dependent or NADH-dependent. Such enzymes are available from a number of sources, including, but not limited to, S. cerevisiae (GenBank Nos: NP_010656, NC_001136, NP_014051, NC_001145), E. coli (GenBank Nos: NP_417484, NC_000913), and C. acetobutylicum (GenBank Nos: NP_349892, NC_003030; NP_349891, NC_003030). U.S. Pat. App. Pub. No. 2009/0269823, which is incorporated herein by reference in its entirety, describes SadB, an alcohol dehydrogenase (ADH) from Achromobacter xylosoxidans. Alcohol dehydrogenases may also include horse liver ADH and Beijerinkia indica ADH, as described by U.S. Pat. App. Pub. No. 2011/0269199, which is incorporated herein by reference in its entirety.

The term “butanol dehydrogenase” refers to a polypeptide (or polypeptides) having enzyme activity that catalyzes the conversion of isobutyraldehyde to isobutanol or the conversion of 2-butanone and 2-butanol. Butanol dehydrogenases are a subset of a broad family of alcohol dehydrogenases. Butanol dehydrogenase may be NAD-dependent or NADP-dependent. The NAD-dependent enzymes are known as EC 1.1.1.1 and are available from, for example, Rhodococcus ruber (GenBank Nos: CAD36475, AJ491307). The NADP-dependent enzymes are known as EC 1.1.1.2 and are available from, for example, Pyrococcus furiosus (GenBank Nos: AAC25556, AF013169). Additionally, a butanol dehydrogenase is available from E. coli (GenBank Nos: NP_417484, NC_000913), and a cyclohexanol dehydrogenase is available from the Acinetobacter species (GenBank Nos: AAG10026, AF282240). The term “butanol dehydrogenase” also refers to an enzyme that catalyzes the conversion of butyraldehyde to 1-butanol, using either NADH or NADPH as a cofactor. Butanol dehydrogenases are available from, for example, C. acetobutylicum (GenBank NOs: NP_149325, NC_001988; NP_349891, NC_003030, NP_349892, and NC_003030) and E. coli (GenBank NOs: NP_417-484 and NC_000913).

The term “branched-chain keto acid dehydrogenase” refers to an enzyme that catalyzes the conversion of α-ketoisovalerate to isobutyryl-CoA (isobutyryl-coenzyme A), typically using NAD+ as an electron acceptor. Example branched-chain keto acid dehydrogenases are known by the EC number 1.2.4.4. Such branched-chain keto acid dehydrogenases are comprised of four subunits and are available from a vast array of microorganisms, including, but not limited to, B. subtilis (GenBank Nos: CAB14336, Z99116; CAB14335, Z99116; CAB14334, Z99116; and CAB14337, Z99116) and Pseudomonas putida (GenBank Nos: AAA65614, M57613; AAA65615, M57613; AAA65617, M57613; and AAA65618, M57613).

The term “pyruvate decarboxylase” refers to an enzyme that catalyzes the decarboxylation of pyruvic acid to acetaldehyde and CO₂. Pyruvate dehydrogenases are known by the EC number 4.1.1.1. These enzymes are found in a number of yeasts, including Saccharomyces cerevisiae (GenBank Nos: CAA97575, CAA97705, CAA97091).

Host cells comprising an isobutanol biosynthetic pathway as provided herein may further comprise one or more additional modifications. U.S. Pat. App. Pub. No. 2009/0305363, which is incorporated herein by reference in its entirety, discloses increased conversion of pyruvate to acetolactate by engineering yeast for expression of a cytosol-localized acetolactate synthase and substantial elimination of pyruvate decarboxylase activity. In some embodiments, the host cells comprise modifications to reduce glycerol-3-phosphate dehydrogenase activity, to disrupt at least one gene encoding a polypeptide having pyruvate decarboxylase activity, and/or to disrupt at least one gene encoding a regulatory element controlling pyruvate decarboxylase gene expression, as described in U.S. Pat. App. Pub. No. 2009/0305363, which is incorporated herein by reference in its entirety. Modifications to a host cell that provide for increased carbon flux through an Entner-Doudoroff Pathway or reducing equivalents balance are described in U.S. Pat. App. Pub. No. 2010/0120105, which is incorporated herein by reference in its entirety. Other modifications include integration of at least one polynucleotide encoding a polypeptide that catalyzes a step in a pyruvate-using biosynthetic pathway.

Other modifications include at least one deletion, mutation, and/or substitution in an endogenous polynucleotide encoding a polypeptide having acetolactate reductase activity. As used herein, “acetolactate reductase activity” refers to the activity of any polypeptide having the ability to catalyze the conversion of acetolactate to 2,3-dihydroxy-2-methyl butyrate (DHMB). DHMB includes “fast DHMB,” which has the 2S, 3S configuration, and “slow DHMB,” which has the 2S, 3R configuration (See Kaneko et al., Phytochemistry 39:115-120 (1995), which refers to “fast DHMB” as anglyceric acid and “slow DHMB” as tiglyceric acid). In some embodiments, the polypeptide having acetolactate reductase activity is YMR226C of Saccharomyces cerevisiae or a homolog thereof.

Additional modifications include a deletion, mutation, and/or substitution in an endogenous polynucleotide encoding a polypeptide having aldehyde dehydrogenase and/or aldehyde oxidase activity, including a polypeptide that catalyzes the oxidation (dehydrogenation) of aldehydes, a polypeptide that catalyzes the conversion of isobutyraldehyde to isobutyric acid, and a polypeptide that corresponds to Enzyme Commission Numbers EC 1.2.1.3, EC 1.2.1.4 or EC 1.2.1.5. Aldehyde oxidases include a polypeptide that catalyzes production of carboxylic acids from aldehydes. Such polypeptides include a polypeptide that catalyzes the conversion of isobutyraldehyde to isobutyric acid and a polypeptide that corresponds to Enzyme Commission Number EC 1.2.3.1. In some embodiments, the polypeptide having aldehyde dehydrogenase activity is ALD6 from Saccharomyces cerevisiae or a homolog thereof.

A genetic modification that has the effect of reducing glucose repression in a PDC-yeast host cell is disclosed in U.S. Pat. App. Pub. No. 2011/0124060, incorporated herein by reference in its entirety. The term “PDC-” refers to a cell that has a genetic modification to inactivate or reduce expression of at least one gene encoding pyruvate decarboxylase (PDC), such that the cell substantially or completely lacks pyruvate decarboxylase enzyme activity. If the yeast cell has more than one expressed (active) PDC gene, then each of the active PDC genes may be inactivated or have minimal expression, thereby producing a PDC-cell. In some embodiments, the pyruvate decarboxylase that is deleted or downregulated is selected from the group consisting of: PDC1, PDC5, PDC6, and combinations thereof. In some embodiments, the pyruvate decarboxylase is selected from PDC1 pyruvate decarboxylase from Saccharomyces cerevisiae, PDC5 pyruvate decarboxylase from Saccharomyces cerevisiae, PDC6 pyruvate decarboxylase from Saccharomyces cerevisiae, pyruvate decarboxylase from Candida glabrata, PDC1 pyruvate decarboxylase from Pichia stipites, PDC2 pyruvate decarboxylase from Pichia stipites, pyruvate decarboxylase from Kluveromyces lactis, pyruvate decarboxylase from Yarrowia lipolytica, pyruvate decarboxylase from Schizosaccharomyces pombe, and pyruvate decarboxylase from Zygosaccharomyces rouxii. In some embodiments, host cells contain a deletion or down-regulation of a polynucleotide encoding a polypeptide that catalyzes the conversion of glyceraldehyde-3-phosphate to glycerate 1,3, bisphosphate. In some embodiments, the enzyme that catalyzes this reaction is glyceraldehyde-3-phosphate dehydrogenase.

Without being bound to a particular theory, in an isobutanologen (PDC-) strain, PDC is deleted, the PDH pathway remains intact, and isobutanol production pathway enzymes are introduced. Often, the first enzyme to act in the isobutanol production pathway is acetolactate synthase (ALS). In isobutanologens, the carbon flux distribution for yeast biomass growth and for the isobutanol pathway under aerobic conditions depends on the relative activity of ALS instead of the PDH enzyme. The physiological behavior of a recombinant isobutanologen is different from an unmodified S. cerevisiae due to the effect of the deletion of PDC genes and introduction of heterologous isobutanol pathway enzymes. To maximize yeast biomass production in a recombinant isobutanologen in aerobic growth phase, the carbon flux should channel through the PDH pathway efficiently to improve yeast biomass yield and minimize carbon flux-to-isobutanol pathway leakages. Pathway leakage products may include isobutanol and isobutyric acid, which may adversely affect the yeast biomass growth rate, and the final yeast biomass achieved. In the production phase, the isobutanol yield and productivity may be adversely affected by accumulation of pathway intermediates (e.g., glycerol and isobutyric acid). Thus, the optimal operating regime (growth and production) for an ethanologen may not be the optimal operating regime for an isobutanologen.

International Pat. Pub. No. WO 2011/103300 discloses recombinant host cells comprising (a) at least one heterologous polynucleotide encoding a polypeptide having dihydroxy-acid dehydratase activity; (b) (i) at least one deletion, mutation, and/or substitution in an endogenous gene encoding a polypeptide affecting Fe—S cluster biosynthesis; and/or (b) (ii) at least one heterologous polynucleotide encoding a polypeptide affecting Fe—S cluster biosynthesis. In some embodiments, the polypeptide affecting Fe—S cluster biosynthesis is encoded by AFT1, AFT2, FRA2, GRX3, or CCC1. In some embodiments, the polypeptide affecting Fe—S cluster biosynthesis is constitutive mutant AFT1 L99A, AFT1 L102A, AFT1 C291F, or AFT1 C293F.

Additionally, host cells may comprise heterologous polynucleotides encoding a polypeptide with phosphoketolase activity and/or a heterologous polynucleotide encoding a polypeptide with phosphotransacetylase activity.

Also, given that accumulation of an alcohol during fermentation may be toxic to some microorganisms, recombinant microorganisms may be engineered to have a greater tolerance for alcohol. Examples of genetic modifications that may improve tolerance to alcohols include, but are not limited to, expression and/or modifications of relA, spoT, and dksA genes (described in U.S. Pat. App. Pub. No. 2009/0203139, incorporated herein by reference in its entirety), elongase genes (Yazawa, et al., Appl. Microbiol. Biotechnol. 91:1593-1600, 2011), heat shock proteins (HSPs), and genes associated with lipid and fatty acid metabolism and cell membrane composition (see, e.g., Ma, et al., Appl. Microbiol. Biotechnol. 87:829-845, 2010). In addition, recombinant microorganisms may be engineered to have a certain level of thermotolerance by, for example, genetically modifying the microorganism to express stress-related genes, such as the genes encoding proteins involved in the ubiquitination process.

In some embodiments, any particular nucleic acid molecule or polypeptide may be at least 80%, or at least 85%, or at least 90%, or at least 95%, or at least 96%, or at least 97%, or at least 98%, or at least 99% identical to a nucleotide sequence or polypeptide sequence described herein.

Standard recombinant DNA and molecular cloning techniques are well known in the art and are described by Sambrook, et al. (Sambrook, J., Fritsch, E. F. and Maniatis, T. (Molecular Cloning: A Laboratory Manual; Cold Spring Harbor Laboratory Press, Cold Spring Harbor, 1989, here in referred to as “Maniatis”) and by Ausubel, et al. (Ausubel, et al., Current Protocols in Molecular Biology, published by Greene Publishing Assoc, and Wiley-Interscience, 1987). Examples of methods to construct microorganisms that comprise a butanol biosynthetic pathway are disclosed, for example, in U.S. Pat. No. 7,851,188 and U.S. Pat. App. Pub. Nos. 2007/0092957; 2007/0259410; 2007/0292927; 2008/0182308; 2008/0274525; 2009/0155870; 2009/0305363; and 2009/0305370, each of which is herein incorporated by reference in its entirety.

Growth and/or Fermentation Media and Carbon Sources

Recombinant microorganism cells disclosed herein are contacted with suitable carbon sources, typically in fermentation media. Suitable carbon sources may include monosaccharides, such as fructose or glucose; disaccharides; oligosaccharides, such as lactose, maltose, galactose, or sucrose; polysaccharides, such as starch or cellulose; ethanol; lactate;

succinate; glycerol; carbon dioxide; methanol; dextrose; xylose; arabinose; methane; amino acids; and mixtures thereof. Suitable carbon sources may also include unpurified mixtures from renewable feedstocks, such as cheese whey permeate, cornsteep liquor, sugar beet molasses, and barley malt. The source of carbon used in the present disclosure may encompass a wide variety of carbon-containing substrates and may be selected based on a number of factors, such as the type of microorganism used. The source of carbon may affect the activity of fermentation enzymes and may be selected to enhance the activity of fermentation enzymes. Without being bound by theory, it is believed that some fermentation enzymes may become active or more active when cells are grown in the presence of dextrose or secondary metabolites thereof.

In some embodiments, the carbon source is selected from glucose, fructose, sucrose, and mixtures thereof, including mixtures thereof with C₈ sugars, such as xylose and/or arabinose, for yeast cells modified to use C₈ sugars. Sucrose may be derived from renewable sugar sources, such as sugar cane, sugar beets, cassava, sweet sorghum, or mixtures thereof. Glucose and dextrose may be derived from renewable grain sources via saccharification of starch-based feedstocks, including grains such as corn, wheat, rye, barley, oats, or mixtures thereof. In addition, fermentable sugars may be derived from renewable cellulosic or lignocellulosic biomass via processes of pretreatment and saccharification, as described, for example, in U.S. Pat. App. Pub. No. 2007/0031918, which is herein incorporated by reference in its entirety.

Biomass, when used in reference to carbon source, refers to any cellulosic or lignocellulosic material and includes materials comprising cellulose and, optionally, further comprising hemicellulose, lignin, starch, oligosaccharides, and/or monosaccharides. Biomass may also comprise additional components, such as protein and/or lipid. Biomass may be derived from a single source, or biomass may comprise a mixture derived from more than one source; for example, biomass may comprise a mixture of corn cobs and corn stover, or a mixture of grass and leaves. Biomass includes, but is not limited to, bioenergy crops, agricultural residues, municipal solid waste, industrial solid waste, sludge from paper manufacture, yard waste, wood, and forestry waste. Examples of biomass include, but are not limited to, corn grain, corn cobs, crop residues such as corn husks, corn stover grasses, wheat, wheat straw, barley, barley straw, hay, rice straw, switchgrass, waste paper, sugar cane bagasse, sorghum, soy, components obtained from milling of grains, trees, branches, roots, leaves, wood chips, sawdust, shrubs and bushes, vegetables, fruits, flowers, animal manure, or mixtures thereof.

In addition to an appropriate carbon source, the growth and/or fermentation media may contain additional components, such as suitable minerals, salts, cofactors, buffers, and other components known to those skilled in the art, suitable for the growth of the cultures and promotion of a selected enzymatic pathway. Like the carbon source, these additional components may affect fermentation and alcohol production and may be selected to increase alcohol production. For example, the growth and/or fermentation media may include a select buffer(s) at a selected concentration(s) to maximize alcohol production.

Optionally, the fermentation media may contain ethanol. In some embodiments, when a recombinant microorganism having a butanol biosynthetic pathway is used as a microorganism for butanol production, supplementation of the fermentation medium with a 2-carbon substrate (e.g., ethanol) may facilitate the survival and growth of the recombinant microorganism. Thus, in some embodiments, ethanol may be supplied to the fermentation medium.

Suitable growth and/or fermentation media include common commercially prepared media such as Luria Bertani (LB) broth, Sabouraud Dextrose (SD) broth, Yeast Medium (YM) broth, broth that includes yeast nitrogen base, ammonium sulfate, and dextrose (as the carbon/energy source), or YPD Medium, a blend of peptone, yeast extract, and dextrose in optimal proportions for growing most Saccharomyces cerevisiae strains. Other defined or synthetic growth and/or fermentation media, such as YPEG medium (a blend of peptone, yeast extract, ethanol, and glycerol), or DMIU medium (a blend of mineral, salts, and dextrose in optimal proportions), may also be used. Media deriving from agricultural sources such as corn thin stillage and corn mash may also be used in growth and/or fermentation. The use of agents known to modulate catabolite repression directly or indirectly, e.g., cyclic adenosine 2′,3′-monophosphate (cAMP), may also be incorporated into the growth and/or fermentation medium.

Growth and/or Fermentation Conditions

Typically, growth and/or fermentation of cells occurs at a temperature in the range of about 20° C. to about 40° C., or about 25° C. to about 35° C., in an appropriate medium. In some embodiments, growth and/or fermentation of cells occurs at a temperature of about 20° C., or about 22° C., or about 25° C., or about 28° C., or about 30° C., or about 32° C., or about 35° C., or about 37° C., or about 40° C. In some aspects, growth and/or fermentation of cells occurs at a temperature of about 20° C. to about 35° C., or about 20° C. to about 30° C., or about 25° C. to about 30° C. Certain cells are more thermo-tolerant and, in such cells, growth and/or fermentation may occur at higher temperatures, such as about 42° C., or about 45° C., or about 47° C., or above 50° C. (e.g., for short periods of time).

Suitable pH for the growth and/or fermentation of cells may range from about pH 3.0 to about pH 9.0, or about pH 3.0 to about pH 7.5. In some embodiments, the pH for the growth and/or fermentation of cells ranges from about pH 4.0 to about pH 9.0, or about pH 4.0 to about pH 8.0. In some aspects, a pH range of about pH 4.0 to about pH 7.0, or about pH 4.0 to about pH 6.0, or about pH 4.5 to about pH 5.5, is used for the fermentation of cells.

Fermentations may be performed under aerobic or anaerobic conditions. In some embodiments, anaerobic or microaerobic conditions are used for fermentation.

### Industrial Batch and Continuous Fermentations

Butanol and other alcohols may be produced using a batch method of fermentation. A classical batch fermentation is a closed system where the composition of the medium is set at the beginning of the fermentation and not subject to artificial alterations during the fermentation. A variation of the standard batch system is the fed-batch system. Fed-batch fermentation processes are also suitable and comprise a typical batch system with the exception that the carbon source or carbon substrate is added in increments as the fermentation progresses. Fed-batch systems are useful when catabolite repression is likely to inhibit the metabolism of the cells and/or when it is desirable to have limited amounts of the carbon source or carbon substrate in the media. Batch and fed-batch fermentations are common and well known in the art, and examples may be found in Thomas D. Brock, Biotechnology: A Textbook of Industrial Microbiology, Second Edition (1989) Sinauer Associates, Inc., Sunderland, Mass., or Deshpande, Mukund V., Appl. Biochem. Biotechnol., 36:227, (1992), herein incorporated by reference in their entirety.

Butanol or other alcohol products may also be produced using continuous fermentation methods. Continuous fermentation is an open system where a defined fermentation medium is added continuously to a bioreactor, and an equal amount of conditioned media is removed simultaneously for processing. Continuous fermentation generally maintains the cultures at a constant high density, where cells are primarily in log phase growth. Continuous fermentation allows for the modulation of one factor or any number of factors that affect cell growth or end product concentration. Methods of modulating nutrients and growth factors for continuous fermentation processes as well as techniques for maximizing the rate of product formation are well known in the art of industrial microbiology.

It is contemplated that the production of butanol or other alcohol products may be practiced using batch, fed-batch, and/or continuous processes and that any known mode of fermentation may be suitable. Additionally, it is contemplated that cells may be immobilized on a substrate as cell catalysts and subjected to fermentation conditions for butanol production.

Methods for Recovering Alcohol from the Fermentation Medium

The bioproduced and/or renewable alcohol, e.g., butanol, may be isolated from the fermentation medium using methods known in the art. For example, solids may be removed from the fermentation medium by centrifugation, filtration, decantation, or the like. Isobutanol may be isolated from the fermentation medium, which may optionally be pre-treated to remove solids as described above, using methods such as distillation (e.g., vacuum distillation), azeotropic distillation, liquid-liquid extraction, adsorption, gas stripping, membrane evaporation, other membrane-based separations, or pervaporation.

Butanol, e.g., isobutanol, may form a low boiling point, azeotropic mixture with water, and distillation may be used to separate the mixture up to its azeotropic composition. Distillation may be used in combination with another separation method to obtain separation around the azeotrope. Methods that may be used in combination with distillation to isolate and purify butanol include, but are not limited to, decantation, liquid-liquid extraction, adsorption, and membrane-based techniques. Additionally, butanol may be isolated using azeotropic distillation using an entrainer.

The butanol-water mixture may form a heterogeneous azeotrope so that distillation may be used in combination with decantation to isolate and purify the butanol. In this method, the butanol-containing fermentation broth is distilled to near the azeotropic composition. Then, the azeotropic mixture is condensed, and the butanol is separated from the fermentation medium by decantation. The decanted aqueous phase may be returned to the first distillation column as reflux. The butanol-rich decanted organic phase may be further purified by distillation in a second distillation column.

The butanol (e.g., isobutanol) may also be isolated from the fermentation medium using liquid-liquid extraction in combination with distillation. In this method, the butanol is extracted from the fermentation broth using liquid-liquid extraction with a suitable solvent. The butanol-containing organic phase is then distilled to separate the butanol from the solvent.

Distillation in combination with adsorption may also be used to isolate butanol (e.g., isobutanol) from the fermentation medium. In this method, the fermentation broth containing the butanol is distilled to near the azeotropic composition and then the remaining water is removed by use of an adsorbent, such as a molecular sieve.

Additionally, distillation in combination with pervaporation may be used to isolate and purify the butanol (e.g., isobutanol) from the fermentation medium. In this method, the fermentation broth containing the butanol is distilled to near the azeotropic composition, and then the remaining water is removed by pervaporation through a hydrophilic membrane.

In situ product removal (ISPR) (also referred to as extractive fermentation) may be used to remove butanol (or other fermentative alcohol) from the fermentation vessel as it is produced, thereby allowing the microorganism to produce butanol at high yields. ISPR may be carried out in a batch mode—where a volume of organic extractant is added to the fermentation vessel and the extractant is not removed during the process—or a continuous mode—where product is continually removed from the reactor. In some instances, ISPR utilizes liquid-liquid extraction. For example, the fermentation medium, which includes the microorganism, may be contacted with an organic extractant (typically before the butanol concentration reaches a toxic level), and the organic extractant and the fermentation medium may form a biphasic mixture. The butanol may partition into the organic extractant phase, decreasing the concentration in the aqueous phase containing the microorganism and thereby limiting the exposure of the microorganism to the inhibitory butanol.

### Further Processing of Renewable Alcohol

Renewable alcohols, such as ethanol and isobutanol, may be sold directly as commodity chemicals. Alternatively, renewable alcohols may be further processed. For example, the renewable alcohols may be dehydrated to their respective olefins (e.g., ethylene and isobutene and one or more renewable linear butenes—typically a mixture of isobutene, 1-butene and cis/trans-2-butene). The renewable ethylene and renewable butenes may then also either be sold directly or still further processed (e.g., separated or reacted) in a variety of different ways to produce a wide variety of renewable hydrocarbon product streams. In some embodiments, further processing may comprise mixing the renewable ethylene and/or butene with ethylene and/or butylene produced by conventional methods (e.g., petroleum cracking) to produce an array of hydrocarbon compounds comprising renewable carbon. Accordingly, such compounds, while not composed solely of renewable carbon, still comprise at least some renewable carbon, with concomitant environmental advantages.

In some embodiments, renewable butene may be produced via the dehydration of renewable isobutanol. The renewable butene formed is typically a tunable mixture of butene isomers, which is easily separated from the isobutanol feed to the dehydration reaction and may be sold directly as a mixture or reacted as a mixture to form other hydrocarbons (e.g., polybutenes). Alternatively, the mixture of renewable butene isomers may be separated (e.g., by distillation, by selective conversion, etc.) into individual butene isomers, which may then be sold individually as feedstocks, polymerized (e.g. to renewable polyisobutylene or butene copolymers), oligomerized (e.g., dimerized, trimerized, etc.) to form higher molecular weight olefins (e.g. isooctene or pentamethylheptenes), isomerized (e.g. isobutene isomerized to linear butenes, 1-butene isomerized to 2-butene, or 2-butene isomerized to 1-butene, etc.), dehydrogenated (e.g. to butadiene), or a combination thereof. In particular, isobutene dimers and trimers may be hydrogenated to provide, e.g., renewable isooctane, which may be useful as a renewable transportation fuel or a renewable additive for transportation fuels.

### EXAMPLES

The following non-limiting examples will further illustrate the systems, methods, and compositions disclosed herein. It should be understood that, while the following examples involve glucose as the carbon source, other carbon sources, feedstocks, or biomass sources, such as corn, may be used for feedstock without departing from the present invention. Moreover, while the following examples involve butanol, other alcohols may be produced without departing from the present invention. It should also be understood that the experimental test methods described below, including the concentrations specified in the experimental test methods, are adapted for bench- or lab-scale production. The experimental test methods described below may be scaled up for commercial/industrial and/or pilot production. The volume of seed fermenter culture and medium may be increased for a pilot-scale or an industrial-scale fermenter. Also, alternative methods of cell preparation may be used for increased quantities of cell mass.

### Experimental Test Methods

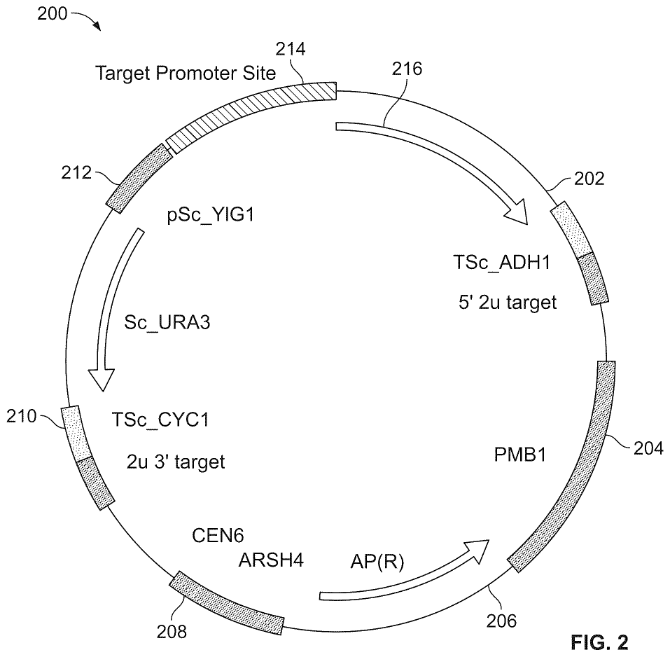

Strain Construction: Each of the promoters to be assessed was introduced into a modified 2-micron plasmid by molecular cloning to control the expression of the pathway enzyme, NADH-dependent ketol-acid reductoisomerase (KARI). FIG. 2 displays an example schematic of a plasmid 200 (e.g., pGV5097 containing 6950 base pairs) used in promoter activity assessment, where areas 202, 204, 206, 208, 210, and 212 represent base pair numbers 1000, 2000, 3000, 4000, 5000, and 6000, respectively. The target promoter 214 was introduced through standard molecular cloning methods directly upstream of the Sel KARI enzyme 216 at the target promoter site. Other plasmid features present in FIG. 2 are well-known to those skilled in the art. Each distinct promoter sequence was added individually and sequenced to verify successful construction and sequence identity. The plasmids were then introduced into test strain GEVO20424 using a standard yeast transformation protocol.

The following promoters were assessed: 1) PSCURA3 (Promoters of the URA3 gene): PSCURA3-541 (SEQ ID NO: 2), PSCURA3-351 (SEQ ID NO: 3), and PSCURA3-227 (SEQ ID NO: 4); 2) PScHSP26 (Promoters of the HSP26 gene): PScHSP26-699 (SEQ ID NO: 6), PScHSP26-495 (SEQ ID NO: 7), PScHSP26-356 (SEQ ID NO: 8), and PScHSP26-226 (SEQ ID NO: 9); 3) PScFIT3 (Promoters of the FIT3 gene): PScFIT3-813 (SEQ ID NO: 11), PScFIT3-650 (SEQ ID NO: 12), PSCFIT3-453 (SEQ ID NO: 13), PScFIT3-338 (SEQ ID NO: 14), and PScFIT3-254 (SEQ ID NO: 15); 4) PSCHXT6 (Promoters of the HXT6 gene): PSCHXT6-881 (SEQ ID NO: 17), PSCHXT6-532 (SEQ ID NO: 18), PSCHXT6-346 (SEQ ID NO: 19), and PSCHXT6-210 (SEQ ID NO: 20); and 5) PSCENO1 (Promoter of the ENO1 gene): PScENO1-726 (SEQ ID NO: 21). Cells of a Saccharomyces cerevisiae strain, GEVO20424, which has been well-characterized in laboratory fermentation experiments, were transformed with the plasmids described above (one plasmid per cell). In all, three different URA3 promoters (SEQ ID Nos: 1-3), four different HSP26 promoters (SEQ ID NOs: 6-9), five different FIT3 promoters (SEQ ID NOs: 11-15), four different HXT6 promoters (SEQ ID NOs: 17-20), and an ENO1 promoter (SEQ ID NO: 21) were introduced individually to generate unique strains.

The resulting transformed strains were then analyzed in shake flask fermentation experiments. In these experiments, each strain was inoculated directly into a 3 mL culture tube containing YPEG from an agar plate and grown at 30° C. with 250 RPM of agitation for 24 hours. Approximately 1 mL of each culture was then added to 25 mL of YPEG in a 150 mL baffled flask to reach a standardized optical density (OD₆₀₀) of 0.2 A.U. These cultures were grown for 24 hours at 30° C. with 250 RPM of agitation, which constitutes the growth or propagation stage. Following this, 4 mL of each culture was added to 71.25 mL of DMIU media containing 120 g/L dextrose or to 71.25 mL of YPD media containing 120 g/L dextrose, and the resulting cultures were grown at 28° C. with 160 RPM of agitation for 42-48 hours, constituting the production or fermentation stage. Samples were taken at relevant times during this process, with cell density (determined by an absorbance reading of OD₆₀₀) being assessed during both the propagation and fermentation stages, and isobutanol content analyzed at several timepoints during the fermentation stage. Isobutanol content was determined using gas chromatography analysis.

Seed Fermentation: A Saccharomyces cerevisiae strain that has deletions in pyruvate decarboxylase genes to restrict the conversion of pyruvate to EtOH and was engineered to produce isobutanol from a carbohydrate source was used as the starting strain. This starting strain was engineered with up to five integrated gene cassettes, each cassette comprising two promoters of interest for controlling the expression of two isobutanol pathway enzymes (SEQ ID NOs: 22-31). These enzymes were chosen for optimal expression of target genes during fermentation and may be modified to tune gene expression.

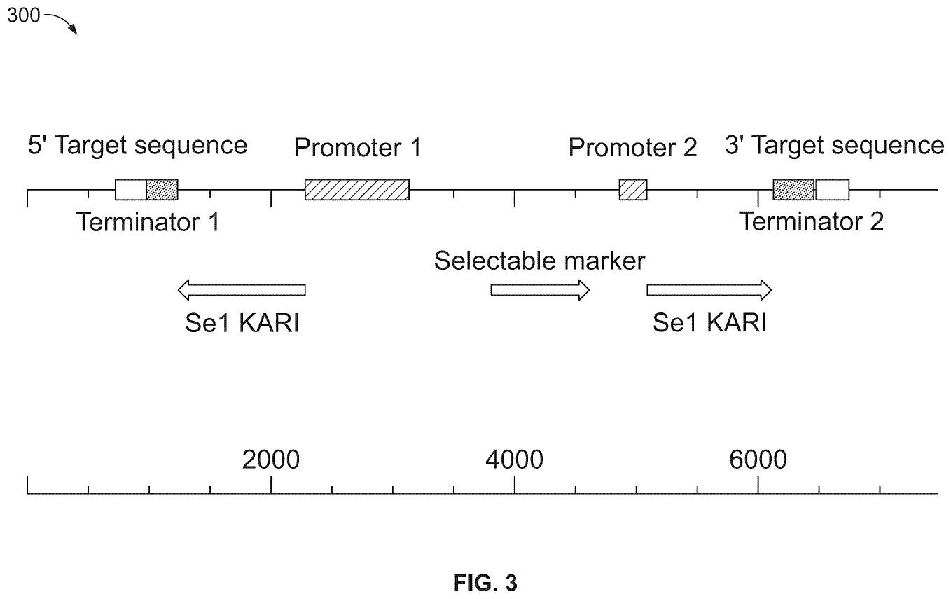

FIG. 3 displays an example schematic of a DNA construct 300 integrated into a yeast genome. Integration of gene cassettes provides a more robust and reliable gene expression profile and is well understood by those skilled in the art. The DNA construct 300 is made from two plasmids that are recombined inside the yeast cell by yeast DNA repair machinery. Two copies of the gene are expressed by unique promoter and terminator sequences. Target sequences at the 5′ end and the 3′ end of the molecule allow for control over where the sequence is integrated into the genome.

For most Seed Fermenter Experiments, the promoters used were PSCURA3 (SEQ ID Nos: 1-4) and PScHSP26 (SEQ ID NOs: 5-9) for controlling the expression of the KARI enzyme (SEQ ID NO: 23), with at least two copies integrated into each strain and four enzymes expressed total. The resulting strains were grown to 6-7 g/L dcw (2.1×10⁸ cells/mL measured by microscopic cell count) in seed fermenters from a frozen culture, which was stored at −80° C. The culture was grown at 28° C., and the fermenter was agitated at 830 rpm. The seed fermenter medium contained 10 g/L yeast extract, 20 g/L peptone, 25 g/L EtOH, and 30 g/L glycerol.

50 mL of the seed fermenter culture was transferred to a second fermenter (1 L). The second fermenter contained 1000 mL of 34% v/v corn mash in distilled water as a medium. In the second fermenter, the yeast culture was grown for about 24 hours to about 32 hours in microaerobic conditions followed by growth in anaerobic conditions for about 6 hours to about 14 hours, at pH 5 (maintained using sodium hydroxide), at a temperature of 28° C., and while being agitated at 300 rpm, producing a batch that included cells and up to about 30 g/L isobutanol. The isobutanol was extracted, and the yeast culture was then harvested and concentrated by centrifugation.

### Example 1. Isobutanol Yield and Production Rates for Selected Promoters

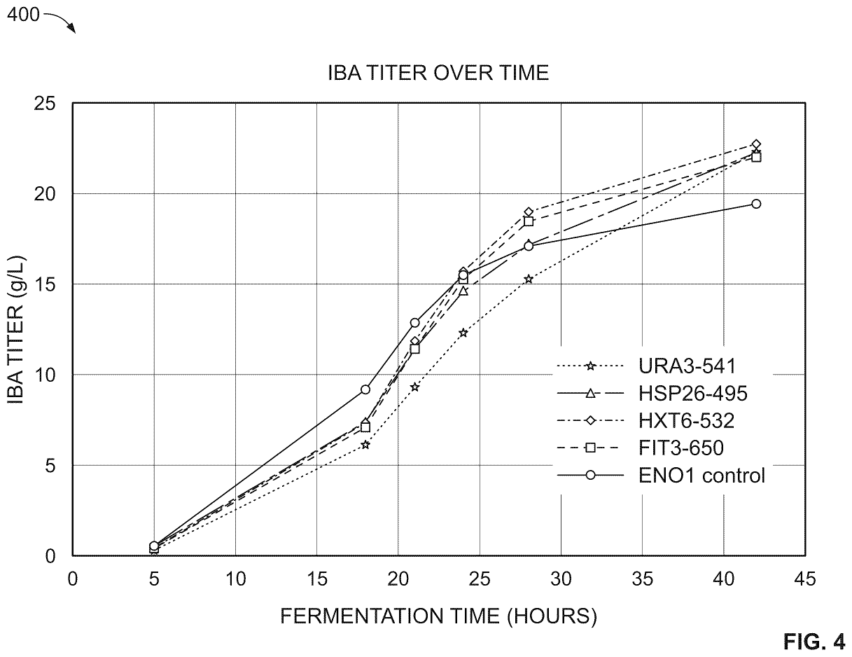

Following shake flask fermentation, the isobutanol titer (g/L) was measured over time for a sample containing cells comprising the following promoters, all made according to the methods described above: ENO1, HSP26, URA3, FIT3, HXT6. FIG. 4 is a graph 400 showing the fermentation isobutanol yield of a recombinant yeast cell comprising a nucleic acid sequence encoding NADH-dependent ketol-acid reductoisomerase (KARI) (SEQ ID NO: 23) operably linked to an HSP26 promoter nucleic acid sequence, PScHSP26-495 (SEQ ID NO: 7), a URA3 promoter nucleic acid sequence, PSCURA3-541 (SEQ ID NO: 2), a FIT3 promoter nucleic acid sequence, PScFIT3-650 (SEQ ID NO: 12), an HXT6 promoter nucleic acid sequence, PSCHXT6-532 (SEQ ID NO:18), or a control ENO1 promoter nucleic acid sequence, PScENO1-726 (SEQ ID NO: 21). The effect of each promoter was determined using shake flask fermentations to generate the yield data, which display that the promoter ENO1 was a faster IBA producer during the first 18-21 hours while other promoters rapidly increased IBA production around 21-28 hours.

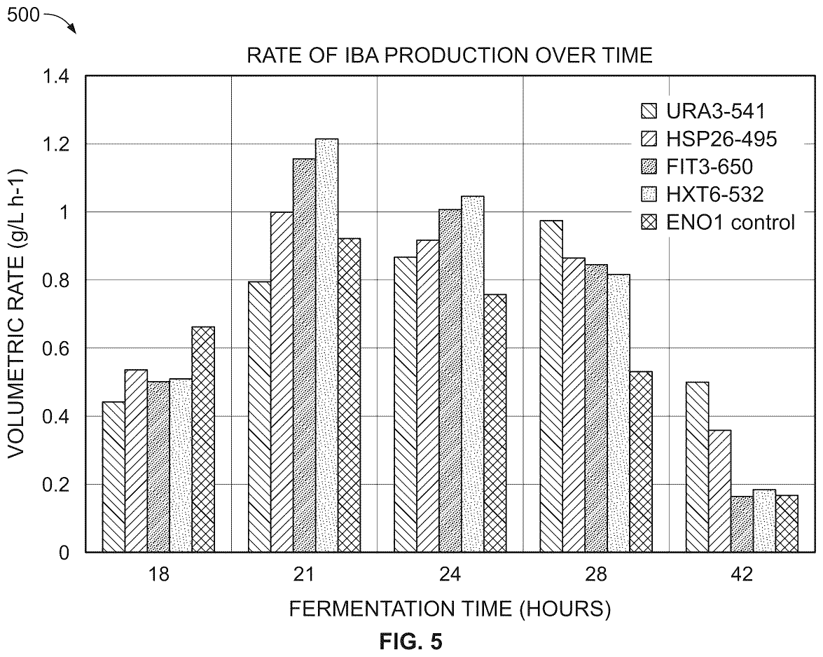

The data shown in FIG. 4 were used to determine rates of isobutanol production at time points between 18 and 42 hours of fermentation for the same five promoter sequences, as displayed in the graph 500 of FIG. 5. All promoters were initially slower to produce isobutanol but increased their rate of production after 18 hours, with some peaking between 21-24 hours and others peaking between 24-28 hours.

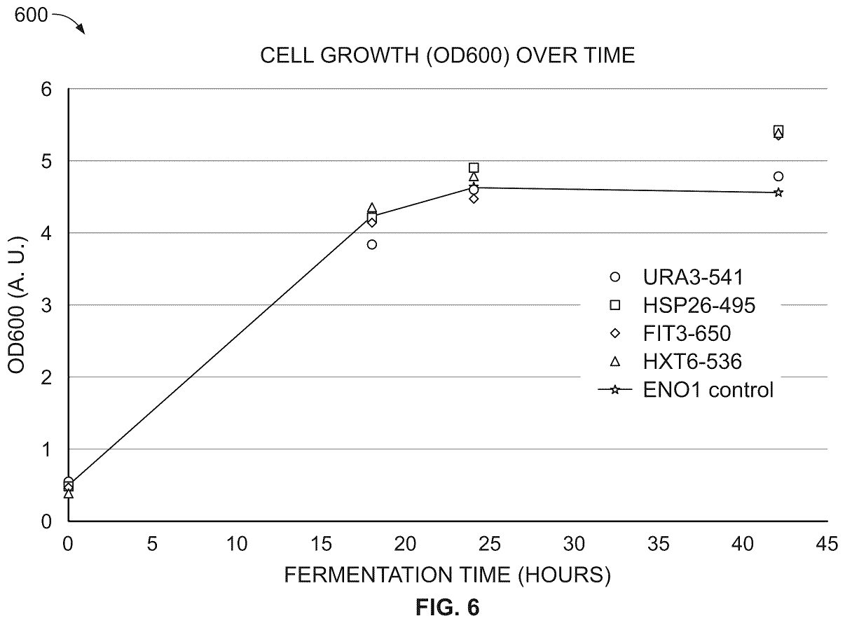

The data shown in FIG. 4 were also used to determine the effect of the same promoters on growth rates of recombinant yeast cells during isobutanol production, as displayed in the graph 600 of FIG. 6. The growth rate of yeast cells producing IBA was determined during fermentation using UV/Vis spectrophotometry. The resulting OD600 measurements demonstrated that all engineered promoters provided a higher cell growth compared to the control ENO1 promoters.

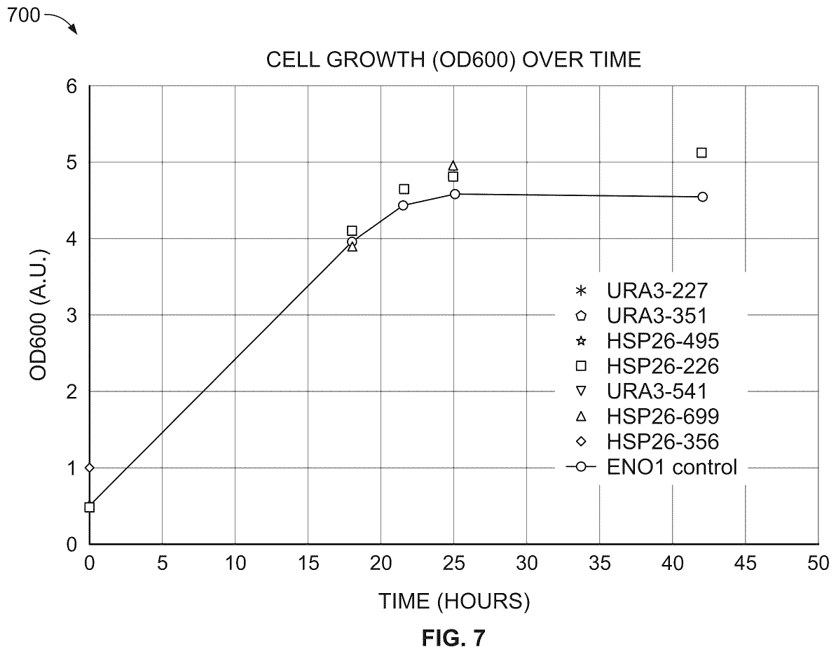

Additional graphs demonstrating the effect of other disclosed promoter sequences on titer, rate of production, and growth rates may be found in FIGS. 7-16.

FIG. 7 displays in a graph 700 the effect of URA3 and HSP26 promoters on growth rates of recombinant yeast during isobutanol production. All newly engineered promoters enabled better cell growth during fermentation than the control ENO1 promoters as assessed by UV/Vis spectrophotometry of fermentation cultures. Cell growth behaviors were very similar among similar promoters, which overlap in the graph 700.

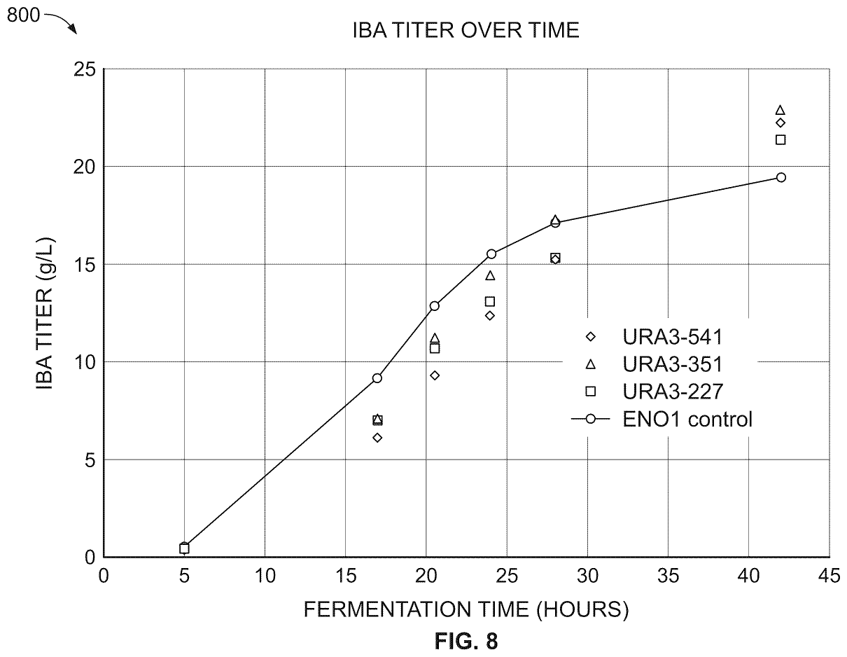

FIG. 8 displays in a graph 800 the effect of PSCURA3 promoters on IBA fermentation over a standard 42-hour shake flask fermentation. While all URA3 promoters initially lagged behind the ENO1 promoter in isobutanol production, the URA3 promoters produced higher endpoint titers than the ENO1 control promoter, consistent with the expected gene expression profile.

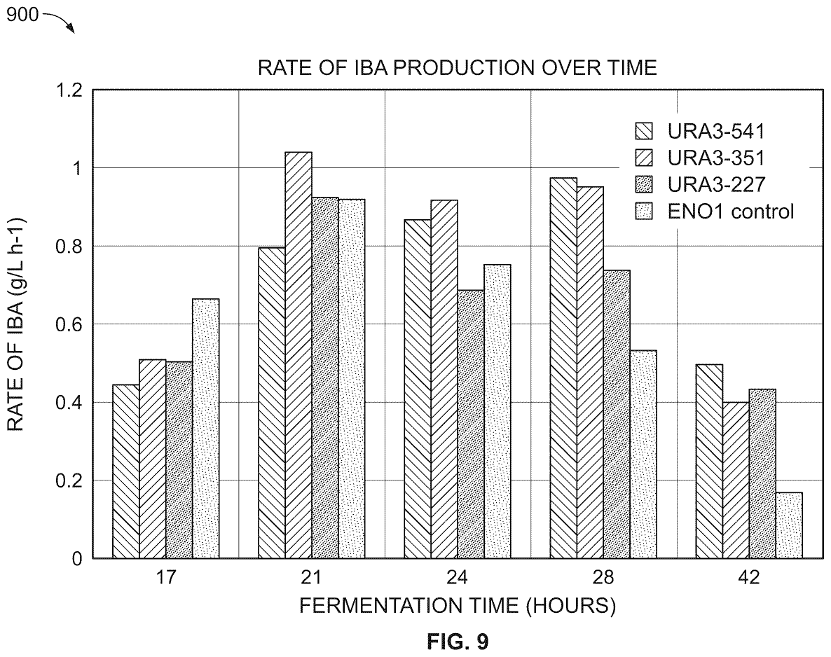

FIG. 9 displays in a graph 900 the rate of isobutanol production during fermentation for FIT3 promoters. All PScFIT3 promoters had a similar production rate as the control at around 17 hours but rapidly increased production between 17 and 28 hours and maintained an overall higher production rate for the remainder of the fermentation.

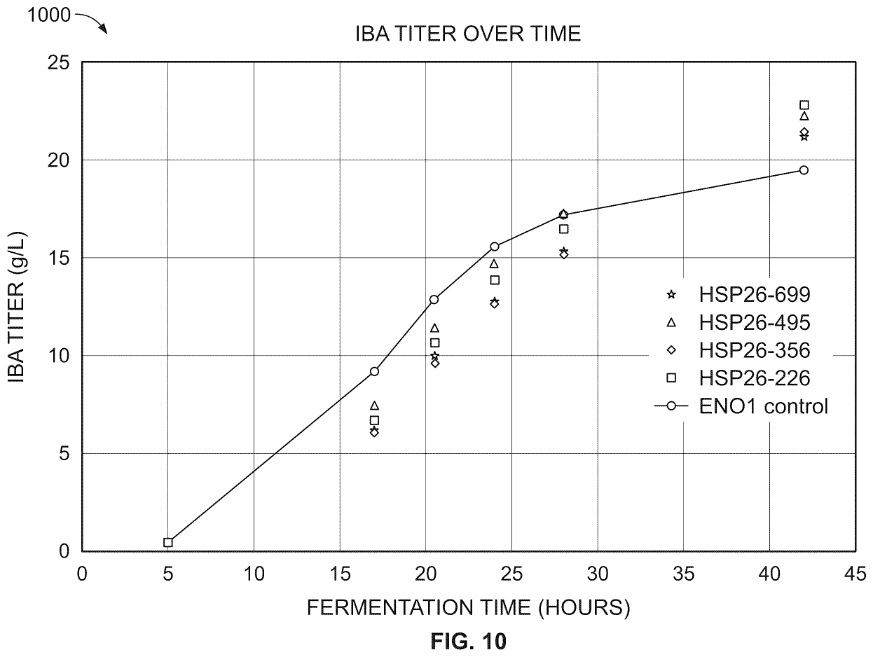

FIG. 10 displays in a graph 1000 the effect of PscHSP26 promoters on isobutanol fermentation over a standard 42-hour shake flask fermentation. While all HSP26 promoters initially lagged behind the ENO1 promoter in isobutanol production, they produced higher endpoint titers than the ENO1 control promoter, consistent with the expected gene expression profile.

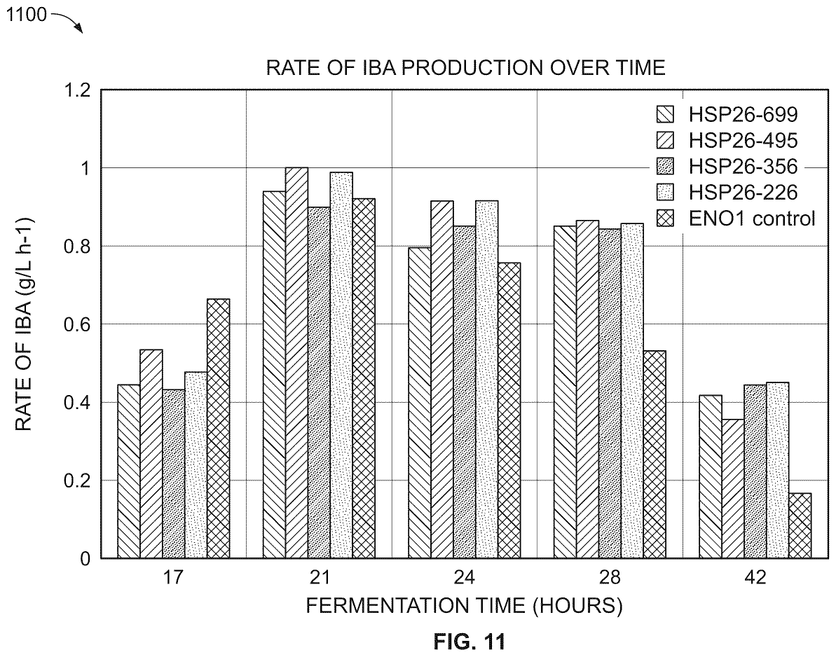

FIG. 11 displays in a graph 1100 the instantaneous rates of IBA production at time points between 18 and 42 hours of fermentation for HSP26 promoters. The HSP26 promoters were initially slower to produce isobutanol but all increased their rate of production by 24 hours to be greater than the control ENO1 promoter.

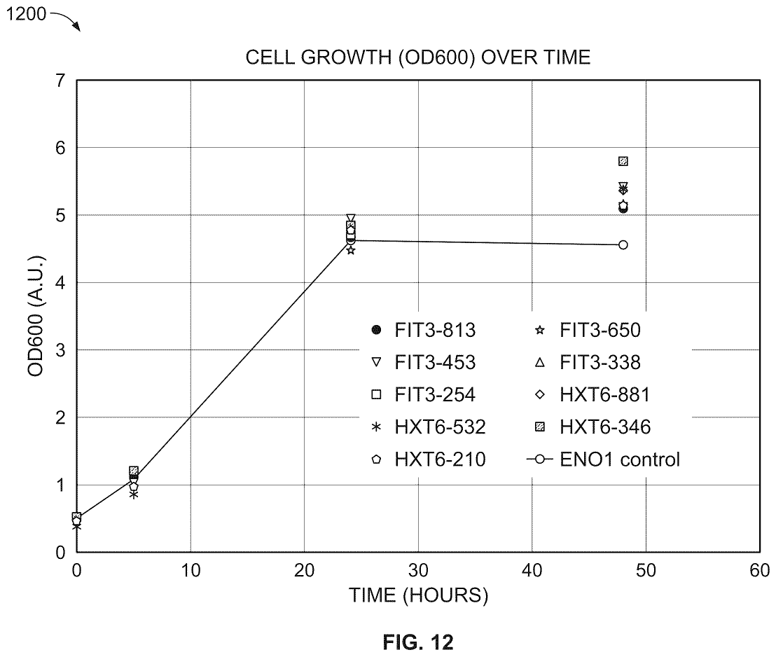

FIG. 12 displays in a graph 1200 the effect of FIT3 and HXT6 promoters on growth rates of recombinant yeast during isobutanol production. The FIT3 and HXT6 promoters enabled somewhat better cell growth during fermentation, especially at later time points, than the control ENO1 promoters as assessed by UV/Vis spectrophotometry of fermentation cultures. Cell growth behaviors were very similar among similar promoters, which overlap in the graph 1200.

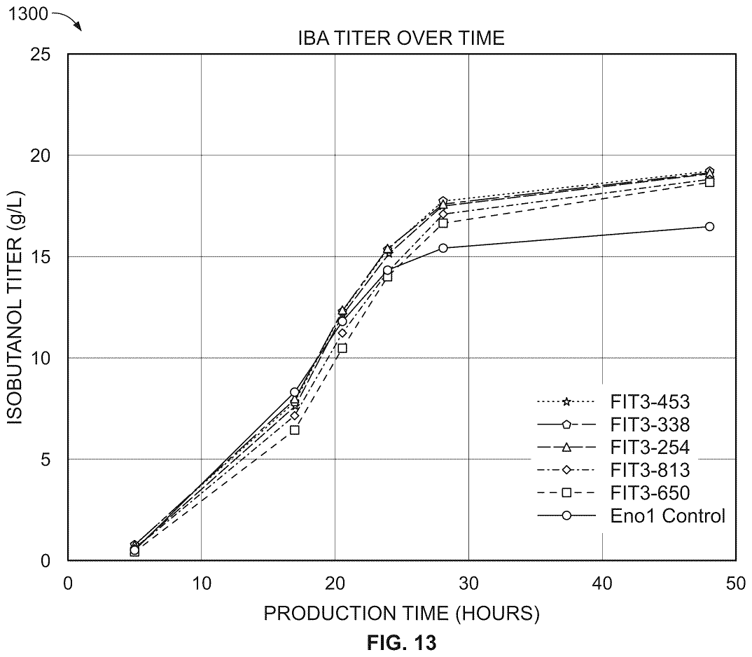

FIG. 13 displays in a graph 1300 the IBA titers for PScFIT3 promoters during isobutanol fermentation. The FIT3 promoters were initially slower to produce isobutanol but only slightly so, having production rates comparable to PSCENO1. All FIT3 promoters increased their rate of production at about 24 hours and maintained robust IBA production throughout the rest of the fermentation.

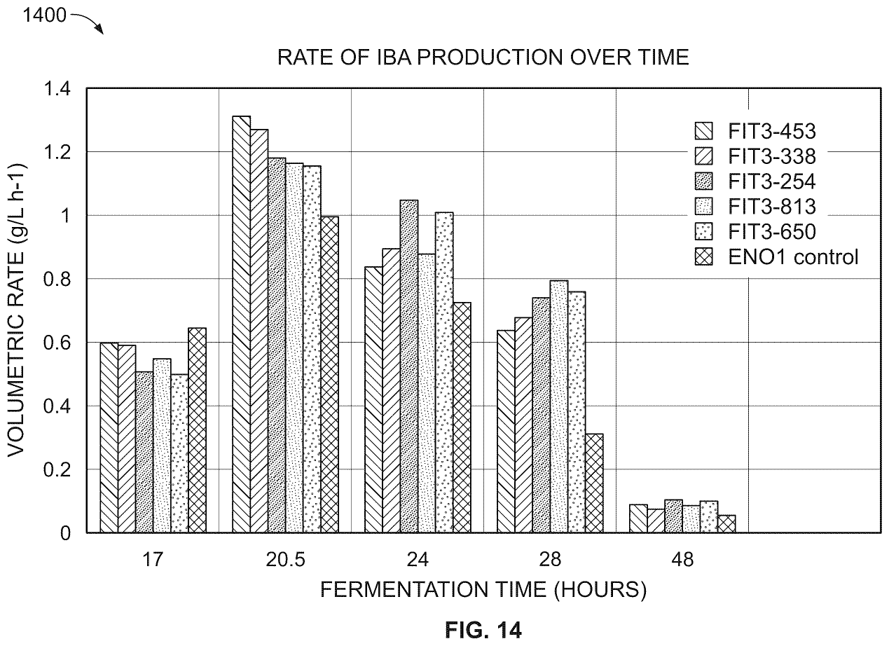

FIG. 14 displays in a graph 1400 the rates of isobutanol production during fermentation for FIT3 promoters. All PScFIT3 promoters had a similar production rate as the control at around 17 hours but rapidly increased production between 17 and 28 hours and maintained an overall higher production rate for the remainder of the fermentation.

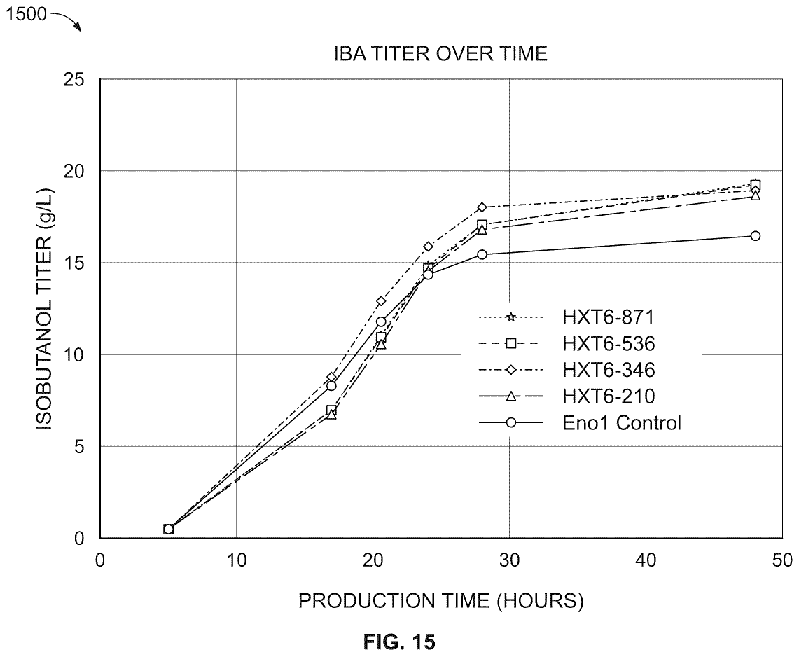

FIG. 15 displays in a graph 1500 the IBA titers for PscHXT6 promoters during isobutanol fermentation. Three of the PscHXT6 promoters were initially slower to produce isobutanol, with PscHXT6-346 having production rates comparable to PScENO1. All HXT6 promoters increased their rate of production at about 20 hours and maintained elevated IBA production throughout the rest of the fermentation.

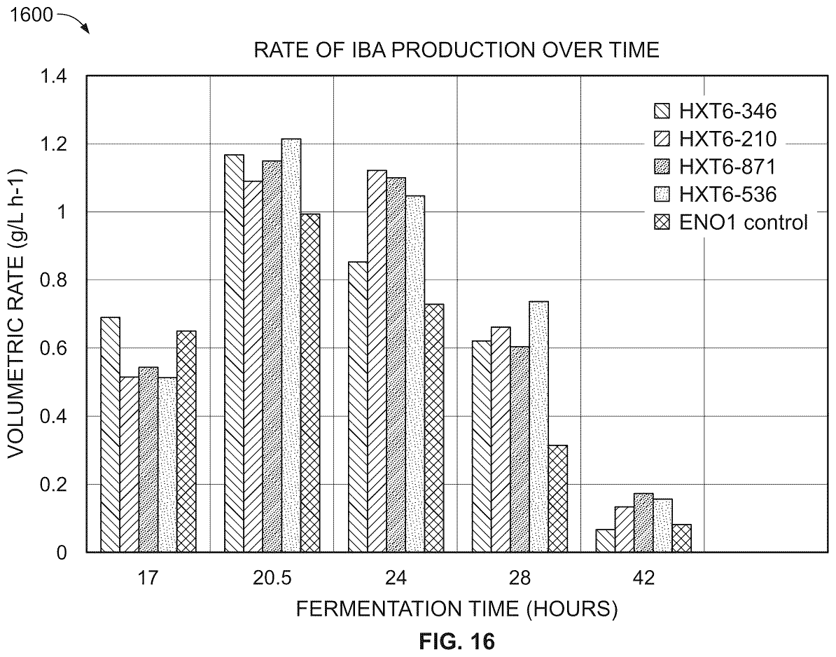

FIG. 16 displays in a graph 1600 the rate of isobutanol production during fermentation for HXT6 promoters. All HXT6 promoters had a similar production rate as the control at around 17 hours, except for HXT6-346, which had IBA production rates even higher than ENO1. All PSCHXT6 promoters rapidly increased production between 17 and 28 hours and maintained an overall higher production rate for the remainder of the fermentation.

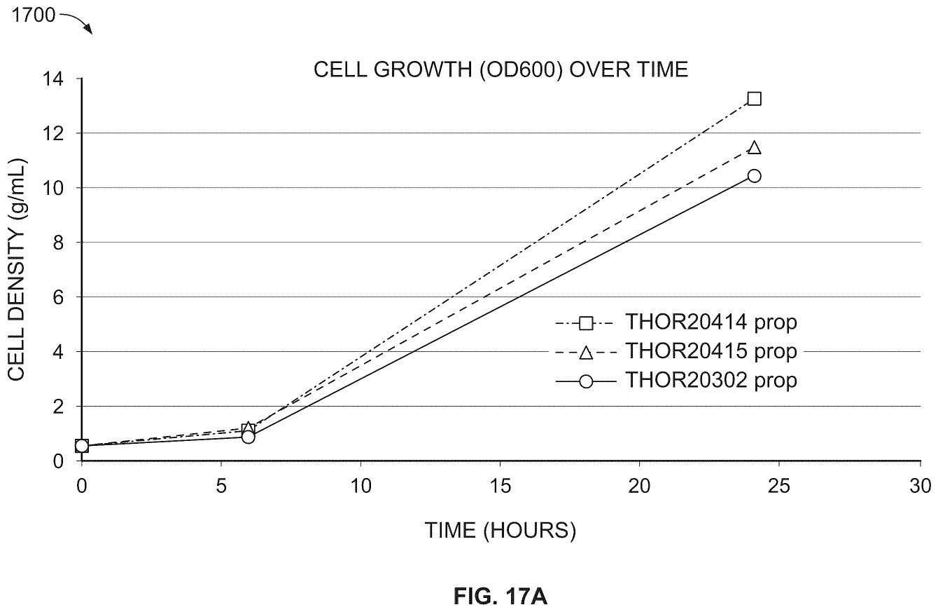

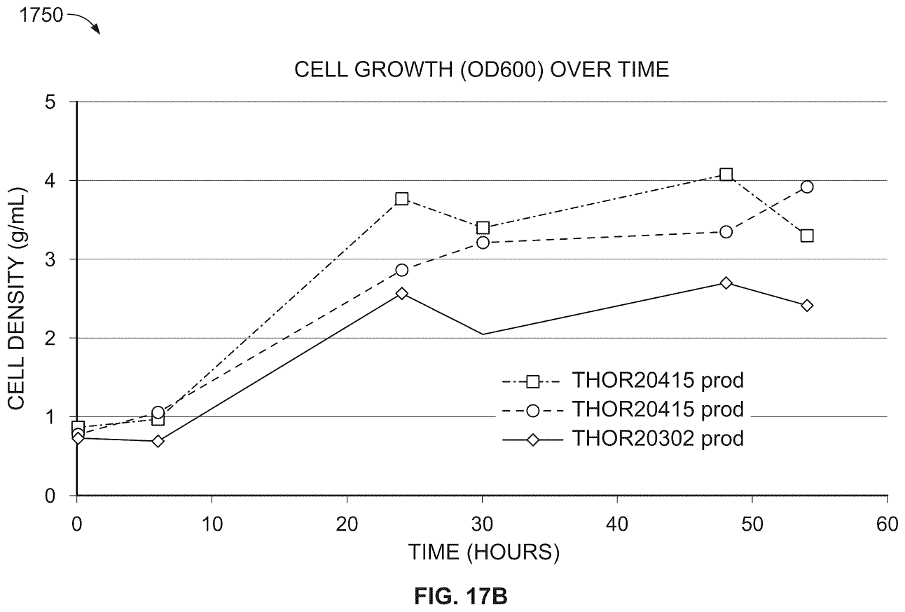

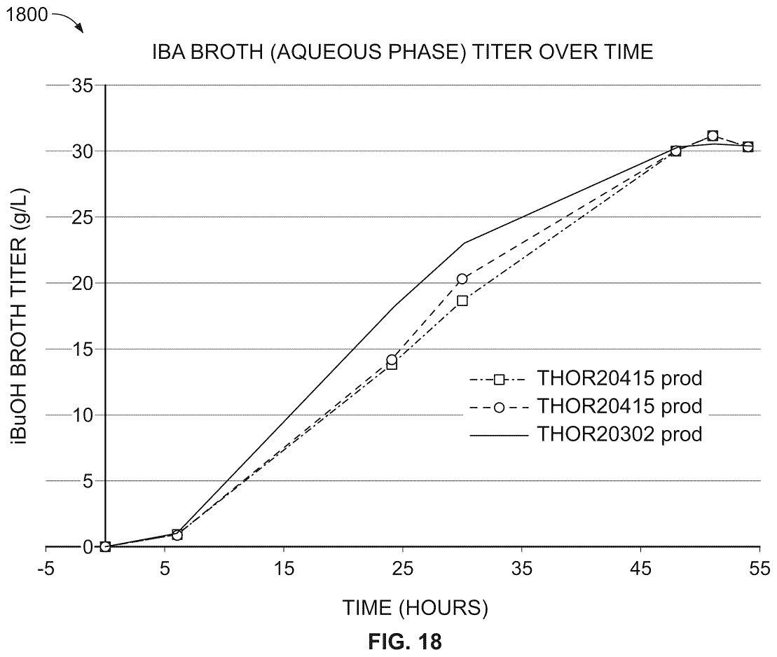

In seed fermentation experiments, three copies of bicistronic gene cassettes were integrated into a butanol pathway-containing yeast strain, and the effect of various promoters was assessed. FIGS. 17 and 18 demonstrate the effect of these promoter sequences in an integrated strain. The results shown in FIGS. 17 and 18 corroborate the promoter efficacy shown in the shake flask experiments.

Namely, FIGS. 17A and 17B display in graphs 1700 and 1750 the growth of recombinant yeast cells in benchtop fermentations with new promoters during propagation (FIG. 17A) and production (FIG. 17B) phases. THOR20414 and THOR20415 were recombinant strains possessing at least three instances of PSCURA3 and three instances of PScHSP26. The use of these promoters over the ENO1 promoter (THOR20302) led to increased growth during propagation and greater increase in growth during fermentation, as predicted.

FIG. 18 displays in a graph 1800 IBA titers during benchtop fermentation for integrated promoter expression cassettes. As shown in shake flask fermentation, the ENO1 promoter strains (THOR20302) were initially much faster in production than the PSCURA3 or PScHSP26 promoter strains (THOR20414 and 20415, genetically identical replicates). Production slowed for THOR20302 after 24 hours, whereas the new promoters increased production rates, particularly around 24-34 hours of fermentation. End point titers were comparable between all three strains.

Without being bound by theory, it is believed that promoters having lengths greater than approximately 200 base pairs exhibit acceptable fermentation stress responses, growth rates, and isobutanol production. The most preferred promoters, e.g., promoters that are most beneficial for cell growth and drive the highest titers, are discussed below.

Therefore, as shown in the graphs of FIGS. 4-6, the ENO1 promoter sequence exhibits high initial activity and high gene expression, both of which decrease toward the end of production. This is consistent with the known properties of the ENO1 promoter, which is activated by the presence of extracellular glucose. The HSP26, URA3, FIT3, and HXT6 promoters exhibit a rapid increase in activity between about 20 hours and about 28 hours after the start of fermentation. Recombinant yeast comprising these promoters exhibited higher initial growth rates, lower initial isobutanol titers, and a rapid increase in isobutanol titer between about 18 hours and about 24 hours after the start of fermentation. Recombinant yeast comprising the FIT3 and HXT6 promoters also exhibited improved growth rates at the beginning of fermentation and increased expression between about 16 hours and about 24 hours after the start of fermentation (FIGS. 12-16). Recombinant yeast comprising the HSP26 and URA3 promoters achieved similar or higher end point titers (FIGS. 8 and 9) as compared to recombinant yeast comprising the ENO1 (sugar-responsive) promoter. Promoters PSCURA3-541 (SEQ ID NO: 2), PSCURA3-351 (SEQ ID NO: 3), PSCURA3-227 (SEQ ID NO: 4), PScHSP26-495 (SEQ ID NO: 7), PScHSP26-699 (SEQ ID NO: 6), PScHSP26-356 (SEQ ID NO: 8), and PScHSP26-226 (SEQ ID NO: 9) exhibited the largest increase in activity between 20 and 28 hours and maintained this productivity the longest time of all the promoter sequences, as shown in FIGS. 10 and 11. As compared to recombinant yeast comprising the ENO1 (sugar-responsive) promoter, recombinant yeast comprising the FIT3 and HXT6 promoters achieved similar results of initially lower titers and comparable or higher end point titers. As discussed above, the data in FIG. 2 and FIGS. 4-6 involve the NADH-dependent ketol-acid reductoisomerase (KARI). Linked to other pathway enzymes, such as 2-ketoisovalerate decarboxylase (KIVD) or acetolactate synthase (ALS), the HSP26, URA3, FIT3, and HXT6 promoters may have affected isobutanol end point titers and production rates differently.

Following the analysis of promoter expression of Sel KARI from a plasmid containing different promoters, three gene cassettes were inserted into a suitable production strain expressing 3 Sel KARI enzymes under control of a URA3 promoter and 3. Sel KARI enzymes under control of a HSP26 promoter, to determine if the desired expression pattern remained consistent across gene expression methods. These strains were run as benchtop fermentation reactions, which tend to be more indicative of the results that may be obtained at industrial scale than shake flask experiments. The strains carrying the integrated copies of the URA3 and HSP26 promoters functioned well within expected parameters, exhibiting initial lower titers but improved growth, an increase in isobutanol production rates between 24 and 34 hours of fermentation, and a similarly high endpoint titer (see FIGS. 17 and 18). These results indicate the suitability of these promoter sequences to promote gene expression at the desired time points. Additional copies of Sel KARI under the control of the URA3 promoter or the HSP26 promoter were expected to increase the end point titer even more. The integration of other pathway enzymes, such as DHAD and KIVD, paired with the URA3 promoter or the HSP26 promoter was expected to further increase yields and cell growth during early stages of production.

Thus, the promoters disclosed herein differentially regulate the expression of genes encoding fermentation enzymes during the propagation and early production phases of the fermentative processes.

The foregoing detailed description has been given for clearness of understanding only and no unnecessary limitations should be understood therefrom as modifications will be obvious to those skilled in the art.

While described in connection with specific embodiments thereof, it will be understood that the principles described herein are capable of further modifications, and this application is intended to cover any variations, uses, or adaptations following, in general, the principles disclosed herein and including such departures from the present disclosure as coming within known or customary practice within the art to which the technology pertains and as may be applied to the essential features hereinbefore set forth and as follows in the scope of the appended claims.

The disclosures, including the claims, figures, and/or drawings, of each and every patent, patent application, and publication cited herein are hereby incorporated herein by reference in their entireties.

### Exemplary Aspects/Embodiments

Certain aspects, including embodiments/aspects of the present subject matter described above, may be beneficial alone or in combination with one or more other aspects recited hereinbelow. In addition, while the present subject matter has been disclosed with reference to certain aspects recited below and in the claims, numerous modifications, alterations, and changes to the described aspects/embodiments are possible without departing from the sphere and scope of the present disclosure. Accordingly, it is intended that the present disclosure is not limited to the described embodiments, aspects, and claims, but that it has the full scope defined by the language of this disclosure and equivalents thereof. While the present technology has been described with reference to the specific aspects/embodiments thereof, it should be understood by those skilled in the art that various changes may be made, and equivalents may be substituted without departing from the true spirit and scope of the disclosure. In addition, many modifications may be made to adapt a particular situation, material, composition of matter, process, and/or process step or steps, to the objective, spirit, and scope of the present disclosure. All such modifications are intended to be within the scope of the claims appended hereto.

Herein below are examples of aspects of the present technology.
A. A method of producing renewable alcohol comprising:
   i) contacting a recombinant host cell comprising a heterologous polynucleotide with a carbon substrate to produce a reaction mixture, the heterologous polynucleotide comprising:
      a) a promoter nucleic acid sequence, wherein the promoter nucleic acid sequence has at least 90% identity to SEQ ID NO: 2, SEQ ID NO: 7, SEQ ID NO: 12, or SEQ ID NO: 18; and
      b) a nucleic acid sequence encoding a biocatalyst peptide operably linked to the promoter nucleic acid sequence;
   ii) fermenting the reaction mixture under a first set of conditions and a second set of conditions to produce a renewable alcohol; and
   iii) optionally recovering the renewable alcohol;
   wherein the second set of conditions differs from the first set of conditions, the nucleic acid sequence encoding the alcohol biosynthetic pathway enzyme is differentially expressed under the first set of conditions than under the second set of conditions, and the recombinant host cell produces alcohol under at least one of the first set of conditions or the second set of conditions.
B. The method according to paragraph A, wherein optionally recovering the renewable alcohol comprises:
   i) combining the reaction mixture and the renewable alcohol with an organic solvent, wherein the renewable alcohol partitions into a non-aqueous organic solvent phase and the reaction mixture partitions into an aqueous phase; and
   ii) distilling the non-aqueous organic solvent phase to separate the renewable alcohol.
C. The method according to any one of paragraphs A-B, wherein the renewable alcohol is recovered before the concentration of the renewable alcohol reaches a level toxic to the recombinant host cell.
D. The method according to any one of paragraphs A-C, wherein the biocatalyst polypeptide is an alcohol biosynthetic pathway enzyme.
E. The method according to paragraph D, wherein the biocatalyst polypeptide is an alcohol biosynthetic pathway enzyme selected from the group consisting of acetolactate synthase (ALS), ketol-acid reductoisomerase (KARI), dihydroxyacid dehydratase (DHAD), 2-keto-acid decarboxylase (KIVD), alcohol dehydrogenase (ADH), and combinations thereof.
F. The method according to any one of paragraphs A-E, wherein the biocatalyst polypeptide comprises a sequence having at least 90% identity to SEQ ID NO: 22, SEQ ID NO: 23, SEQ ID NO: 24, SEQ ID NO: 25, or SEQ ID NO: 26.
G. The method according to any one of paragraphs A-F, wherein the renewable alcohol is butanol.
H. The method according to any one of paragraphs A-G, wherein the renewable alcohol is isobutanol.
I. The method according to any one of paragraphs A-H, wherein the recombinant host cell is a recombinant yeast cell.
J. The method according to paragraph I, wherein the yeast is selected from the group consisting of Saccharomyces, Kluyveromyces, Schizosaccharomyces, and combinations thereof.
K. The method according to any one of paragraphs A-J, wherein the first set of conditions and the second set of conditions differ in at least one of a source of the carbon substrate, a concentration of dissolved oxygen, a temperature, a pH, or a concentration of the renewable alcohol.
L. The method according to any one of paragraphs A-K, wherein the rate of renewable alcohol production is less under the first set of conditions than under the second set of conditions.
M. The method according to any one of paragraphs A-L, wherein the expression of the nucleic acid sequence encoding the biocatalyst peptide is higher in a production phase of fermentation than in a propagation phase of fermentation.
N. A method of producing butanol comprising:
   i) contacting a recombinant host cell comprising a heterologous polynucleotide with a carbon substrate to produce a reaction mixture, the heterologous polynucleotide comprising:
      a) a promoter nucleic acid sequence, wherein the promoter nucleic acid sequence has at least 90% identity to SEQ ID NO: 2, SEQ ID NO: 7, SEQ ID NO: 12, or SEQ ID NO: 18; and
      b) a nucleic acid sequence encoding an enzyme operably linked to the promoter nucleic acid sequence, wherein the enzyme is selected from the group consisting of acetolactate synthase (ALS), ketol-acid reductoisomerase (KARI), dihydroxyacid dehydratase (DHAD), 2-keto-acid decarboxylase (KIVD), alcohol dehydrogenase (ADH), and mixtures thereof;
   ii) fermenting the reaction mixture under a first set of conditions and a second set of conditions to produce butanol; and
   iii) optionally recovering the butanol,
   wherein the second set of conditions differs from the first set of conditions, the nucleic acid sequence encoding the enzyme is differentially expressed under the first set of conditions than under the second set of conditions, and the recombinant host cell produces butanol under at least one of the first set of conditions or the second set of conditions.
O. The method according to paragraph N, wherein the recombinant host cell is a recombinant yeast cell.
P. The method according to paragraph O, wherein the yeast is selected from the group consisting of Saccharomyces, Kluyveromyces, Schizosaccharomyces, and combinations thereof.
Q. The method according to any one of paragraphs N-P, wherein the first set of conditions and the second set of conditions differ in at least one of a source of the carbon substrate, a concentration of dissolved oxygen, a temperature, a pH, or a concentration of the butanol.
R. The method according to any one of paragraphs N-Q, wherein the rate of butanol production is less under the first set of conditions than under the second set of conditions.
S. The method according to any one of paragraphs N—R, wherein the expression of the nucleic acid sequence encoding the enzyme is higher in a production phase of fermentation than in a propagation phase of fermentation.
T. An isolated polynucleotide comprising:
   (a) a promoter nucleic acid sequence, wherein the promoter nucleic acid sequence has at least 90% identity to SEQ ID NO: 2, SEQ ID NO: 7, SEQ ID NO: 12, or SEQ ID NO: 18; and
   (b) a nucleic acid sequence encoding an alcohol biosynthetic pathway enzyme operably linked to the promoter nucleic acid sequence.
U. A recombinant host cell comprising the isolated polynucleotide of paragraph T.
V. The recombinant host cell according to paragraph U, wherein the recombinant host cell is a recombinant microbial cell.
W. The recombinant host cell according to paragraph U, wherein the recombinant host cell is a recombinant yeast cell.
X. The recombinant host cell according to paragraph W, wherein the yeast is selected from the group consisting of Saccharomyces, Kluyveromyces, Schizosaccharomyces, and combinations thereof.
Y. A composition for producing a renewable alcohol comprising the recombinant host cell according to any one of paragraphs U-X.
Z. A method of producing a renewable alcohol, the method comprising:
   i) mixing the composition of paragraph Y with a carbon substrate to produce a reaction mixture;
   ii) fermenting the reaction mixture to produce alcohol; and,
   iii) optionally recovering the renewable alcohol.
AA. A multiphase fermentation composition comprising:
   i) an aqueous phase comprising the reaction mixture of paragraph Z; and
   ii) a non-aqueous organic solvent phase comprising the renewable alcohol and an organic solvent.
BB. The multiphase fermentation composition of paragraph AA, wherein the organic solvent is selected from the group consisting of phenetole, octanol, heptanol, oleyl alcohol, and mixtures thereof.

What is claimed is:

1. A method of producing renewable alcohol comprising:
a) contacting a recombinant host cell comprising a heterologous polynucleotide with a carbon substrate to produce a reaction mixture, the heterologous polynucleotide comprising:
i) a promoter nucleic acid sequence, wherein the promoter nucleic acid sequence has at least 90% identity to SEQ ID NO: 2, SEQ ID NO: 7, SEQ ID NO: 12, or SEQ ID NO: 18; and
ii) a nucleic acid sequence encoding a biocatalyst peptide operably linked to the promoter nucleic acid sequence;
b) fermenting the reaction mixture under a first set of conditions and a second set of conditions to produce a renewable alcohol; and
c) optionally recovering the renewable alcohol,
wherein the second set of conditions differs from the first set of conditions, the nucleic acid sequence encoding the alcohol biosynthetic pathway enzyme is differentially expressed under the first set of conditions than under the second set of conditions, and the recombinant host cell produces alcohol under at least one of the first set of conditions or the second set of conditions.

2. The method according to claim 1, wherein recovering the renewable alcohol comprises:
a) combining the reaction mixture and the renewable alcohol with an organic solvent, wherein the renewable alcohol partitions into a non-aqueous organic solvent phase and the reaction mixture partitions into an aqueous phase; and
b) distilling the non-aqueous organic solvent phase to separate the renewable alcohol.

3. The method according to claim 2, wherein the renewable alcohol is recovered before the concentration of the renewable alcohol reaches a level toxic to the recombinant host cell.

4. The method according to claim 1, wherein the biocatalyst polypeptide is an alcohol biosynthetic pathway enzyme.

5. The method according to claim 4, wherein the alcohol biosynthetic pathway enzyme is selected from the group consisting of acetolactate synthase (ALS), ketol-acid reductoisomerase (KARI), dihydroxyacid dehydratase (DHAD), 2-keto-acid decarboxylase (KIVD), alcohol dehydrogenase (ADH), and combinations thereof.

6. The method according to claim 1, wherein the biocatalyst polypeptide comprises a sequence having at least 90% identity to SEQ ID NO: 22, SEQ ID NO: 23, SEQ ID NO: 24, SEQ ID NO: 25, or SEQ ID NO: 26.

7. The method according to claim 1, wherein the renewable alcohol is isobutanol.

8. The method according to claim 1, wherein the recombinant host cell is a recombinant yeast cell selected from the group consisting of recombinant Saccharomyces, recombinant Kluyveromyces, recombinant Schizosaccharomyces, and combinations thereof.

9. The method according to claim 1, wherein the first set of conditions and the second set of conditions differ in at least one of a source of the carbon substrate, a concentration of dissolved oxygen, a temperature, a pH, or a concentration of the renewable alcohol.

10. A method of producing butanol comprising:
a) contacting a recombinant host cell comprising a heterologous polynucleotide with a carbon substrate to produce a reaction mixture, the heterologous polynucleotide comprising:
i) a promoter nucleic acid sequence, wherein the promoter nucleic acid sequence has at least 90% identity to SEQ ID NO: 2, SEQ ID NO: 7, SEQ ID NO: 12, or SEQ ID NO: 18; and
ii) a nucleic acid sequence encoding an enzyme operably linked to the promoter nucleic acid sequence, wherein the enzyme is selected from the group consisting of acetolactate synthase (ALS), ketol-acid reductoisomerase (KARI), dihydroxyacid dehydratase (DHAD), 2-keto-acid decarboxylase (KIVD), alcohol dehydrogenase (ADH), and mixtures thereof;
b) fermenting the reaction mixture under a first set of conditions and a second set of conditions to produce butanol; and
c) optionally recovering the butanol;
wherein the second set of conditions differs from the first set of conditions, the nucleic acid sequence encoding the enzyme is differentially expressed under the first set of conditions than under the second set of conditions, and the recombinant host cell produces butanol under at least one of the first set of conditions or the second set of conditions.

11. The method according to claim 10, wherein the recombinant host cell is a recombinant yeast cell selected from the group consisting of recombinant Saccharomyces, recombinant Kluyveromyces, recombinant Schizosaccharomyces, and combinations thereof.

12. The method according to claim 10, wherein the first set of conditions and the second set of conditions differ in at least one of a source of the carbon substrate, a concentration of dissolved oxygen, a temperature, a pH, or a concentration of the butanol.

13. The method according to claim 10, wherein the rate of butanol production is less under the first set of conditions than under the second set of conditions.

14. The method according to claim 10, wherein the expression of the nucleic acid sequence encoding the enzyme is higher in a production phase of fermentation than in a propagation phase of fermentation.

15. An isolated polynucleotide comprising:
(a) a promoter nucleic acid sequence, wherein the promoter nucleic acid sequence has at least 90% identity to SEQ ID NO: 2, SEQ ID NO: 7, SEQ ID NO: 12, or SEQ ID NO: 18; and
(b) a nucleic acid sequence encoding an alcohol biosynthetic pathway enzyme operably linked to the promoter nucleic acid sequence.

16. A recombinant yeast cell comprising the isolated polynucleotide of claim 13.

17. A composition for producing a renewable alcohol comprising the recombinant yeast cell according to claim 14.

18. A method of producing a renewable alcohol, the method comprising:
a) mixing the composition of claim 15 with a carbon substrate to produce a reaction mixture;
b) fermenting the reaction mixture to produce a renewable alcohol; and
c) optionally recovering the alcohol.

19. A multiphase fermentation composition comprising:
a) an aqueous phase comprising the reaction mixture of claim 16; and
b) a non-aqueous organic solvent phase comprising the renewable alcohol and an organic solvent.

20. The multiphase fermentation composition of claim 17, wherein the organic solvent is selected from the group consisting of phenetole, octanol, heptanol, oleyl alcohol, and mixtures thereof.


SEQUENCE LISTING (36 sequences)

SEQ ID NO: 1 - DNA, 1000 bp, Saccharomyces cerevisiae
  source 1..1000: mol_type = genomic DNA
  regulatory 1..1000: regulatory_class = promoter
tcataaaattgataaggagaatccatacaagaaatcgatcgaattatcattgaaatccattcaactaacatcacacttgctggaagataataacgatctgcgtaaaaacgagatattcgctattattcaagctttggcacatcaatgcatcaatccgtgtaagcagataagtgaatttgcagtggtaacgctagagcagacgctcatcaataaaatcgaaattccaactaatgagatggaatcggtagaagaattaattgagggcggattactaccgttgctaaattcgagtgaaacacaggaagaccagaaaatcctcatttcatccatattaacaataatttcaaatgtttatttgcattatttgaaactagggaagacaagcaacgaaacgtttttgaaaattttgagtattttcaataaatttgtagaggactcagatattgaaaaaaagctacagcaattaatacttgataagaagagtattgagaagggcaacggttcatcatctcatggatctgcacatgaacaaacaccagagtcaaacgacgttgaaattgaggctactgcgccaattgatgacaatacagacgatgataacaaaccgaagttatctgatgtagaaaaggattaaagatgctaagagatagtgatgatatttcataaataatgtaattctatatatgttaattaccttttttgcgaggcatatttatggtgaaggataagttttgaccatcaaagaaggttaatgtggctgtggtttcagggtccataaagcttttcaattcatcttttttttttttgttcttttttttgattccggtttctttgaaatttttttgattcggtaatctccgagcagaaggaagaacgaaggaaggagcacagacttagattggtatatatacgcatatgtggtgttgaagaaacatgaaattgcccagtattcttaacccaactgcacagaacaaaaacctgcaggaaacgaagataaatc

SEQ ID NO: 2 - DNA, 541 bp, Saccharomyces cerevisiae
  source 1..541: mol_type = genomic DNA
  regulatory 1..541: regulatory_class = promoter
gcaattaatacttgataagaagagtattgagaagggcaacggttcatcatctcatggatctgcacatgaacaaacaccagagtcaaacgacgttgaaattgaggctactgcgccaattgatgacaatacagacgatgataacaaaccgaagttatctgatgtagaaaaggattaaagatgctaagagatagtgatgatatttcataaataatgtaattctatatatgttaattaccttttttgcgaggcatatttatggtgaaggataagttttgaccatcaaagaaggttaatgtggctgtggtttcagggtccataaagcttttcaattcatcttttttttttttgttcttttttttgattccggtttctttgaaatttttttgattcggtaatctccgagcagaaggaagaacgaaggaaggagcacagacttagattggtatatatacgcatatgtggtgttgaagaaacatgaaattgcccagtattcttaacccaactgcacagaacaaaaacctgcaggaaacgaagataaatc

SEQ ID NO: 3 - DNA, 351 bp, Saccharomyces cerevisiae
  source 1..351: mol_type = genomic DNA
  regulatory 1..351: regulatory_class = promoter
gtgatgatatttcataaataatgtaattctatatatgttaattaccttttttgcgaggcatatttatggtgaaggataagttttgaccatcaaagaaggttaatgtggctgtggtttcagggtccataaagcttttcaattcatcttttttttttttgttcttttttttgattccggtttctttgaaatttttttgattcggtaatctccgagcagaaggaagaacgaaggaaggagcacagacttagattggtatatatacgcatatgtggtgttgaagaaacatgaaattgcccagtattcttaacccaactgcacagaacaaaaacctgcaggaaacgaagataaatc

SEQ ID NO: 4 - DNA, 227 bp, Saccharomyces cerevisiae
  source 1..227: mol_type = genomic DNA
  regulatory 1..227: regulatory_class = promoter
cataaagcttttcaattcatcttttttttttttgttcttttttttgattccggtttctttgaaatttttttgattcggtaatctccgagcagaaggaagaacgaaggaaggagcacagacttagattggtatatatacgcatatgtggtgttgaagaaacatgaaattgcccagtattcttaacccaactgcacagaacaaaaacctgcaggaaacgaagataaatc

SEQ ID NO: 5 - DNA, 1000 bp, Saccharomyces cerevisiae
  source 1..1000: mol_type = genomic DNA
  regulatory 1..1000: regulatory_class = promoter
cgttggactttttttaatataacctaccataggacacactttgttgttgatgttggacaattcgttaattaagagtccctaaacggctctactagttccaacctcactttgttttttcatttttttatgttttttctagaaccttctttacgtgattctcgctcggaatccgtcaatagaatgttttcagtctccgtttcaatattctgcgcacatcaatcattttcttactacatacactaacattactcctagtttaatttaattgaatttttaactttcttttcttttcatttggcaatttggctccttgaaaacaagactatgggtctctctcataagcctcagggggggaccccaaaaaaataacgcggccatcttgcatgcaccgttgaacctgtagcttacagtaagccacaattctcttaccttcttggcaatgtggcacaaaataatctggttatgtgtcttcatttggtaatcactgggatgttactggggcagcagcaactccgtgtgtacccctaactccgtgtgtacccctaaagaaccttgcctgtcaaggtgcattgttggatcggaatagtaaccgtctttacatgaacatccacaaccaacgaaagtgctttttcaagcattgcttgatttctagaaagatcgatggttattccctcccccttatgcgtccaaaaatatagggtgctcgtaacagtaaggtattcgcacttagcgtgctcgcaacacaaaattaagtaatatgcgagttttagatgtccttgcggatctatgcacgttcttgagtggtatttcataacaacggttctttttcacccttattcctaaacatataaataggacctccattagttagagatctgtttttaatccattcacctttcattctactctcttatactaataaaaccaccgataaagatatatcagatctctattaaaacaggtatccaaaaaagcaaacaaacaaactaaacaaattaac

SEQ ID NO: 6 - DNA, 699 bp, Saccharomyces cerevisiae
  source 1..699: mol_type = genomic DNA
  regulatory 1..699: regulatory_class = promoter
tttggctccttgaaaacaagactatgggtctctctcataagcctcagggggggaccccaaaaaaataacgcggccatcttgcatgcaccgttgaacctgtagcttacagtaagccacaattctcttaccttcttggcaatgtggcacaaaataatctggttatgtgtcttcatttggtaatcactgggatgttactggggcagcagcaactccgtgtgtacccctaactccgtgtgtacccctaaagaaccttgcctgtcaaggtgcattgttggatcggaatagtaaccgtctttacatgaacatccacaaccaacgaaagtgctttttcaagcattgcttgatttctagaaagatcgatggttattccctcccccttatgcgtccaaaaatatagggtgctcgtaacagtaaggtattcgcacttagcgtgctcgcaacacaaaattaagtaatatgcgagttttagatgtccttgcggatctatgcacgttcttgagtggtatttcataacaacggttctttttcacccttattcctaaacatataaataggacctccattagttagagatctgtttttaatccattcacctttcattctactctcttatactaataaaaccaccgataaagatatatcagatctctattaaaacaggtatccaaaaaagcaaacaaacaaactaaacaaattaac

SEQ ID NO: 7 - DNA, 495 bp, Saccharomyces cerevisiae
  source 1..495: mol_type = genomic DNA
  regulatory 1..495: regulatory_class = promoter
ggatgttactggggcagcagcaactccgtgtgtacccctaaagaaccttgcctgtcaaggtgcattgttggatcggaatagtaaccgtctttacatgaacatccacaaccaacgaaagtgctttttcaagcattgcttgatttctagaaagatcgatggttattccctcccccttatgcgtccaaaaatatagggtgctcgtaacagtaaggtattcgcacttagcgtgctcgcaacacaaaattaagtaatatgcgagttttagatgtccttgcggatctatgcacgttcttgagtggtatttcataacaacggttctttttcacccttattcccaaacatataaataggacctccattagttagagatctgtttttaatccattcacctttcattctactctcttatactaataaaaccaccgataaagatatatcagatctctattaaaacaggtatccaaaaaagcaaacaaacaaactaaacaaattaac

SEQ ID NO: 8 - DNA, 356 bp, Saccharomyces cerevisiae
  source 1..356: mol_type = genomic DNA
  regulatory 1..356: regulatory_class = promoter
atttctagaaagatcgatggttattccctcccccttatgcgtccaaaaatatagggtgctcgtaacagtaaggtattcgcacttagcgtgctcgcaacacaaaattaagtaatatgcgagttttagatgtccttgcggatctatgcacgttcttgagtggtatttcataacaacggttctttttcacccttattcccaaacatataaataggacctccattagttagagatctgtttttaatccattcacctttcattctactctcttatactaataaaaccaccgataaagatatatcagatctctattaaaacaggtatccaaaaaagcaaacaaacaaactaaacaaattaac

SEQ ID NO: 9 - DNA, 226 bp, Saccharomyces cerevisiae
  source 1..226: mol_type = genomic DNA
  regulatory 1..226: regulatory_class = promoter
ccttgcggatctatgcacgttcttgagtggtatttcataacaacggttctttttcacccttattcccaaacatataaataggacctccattagttagagatctgtttttaatccattcacctttcattctactctcttatactaataaaaccaccgataaagatatatcagatctctattaaaacaggtatccaaaaaagcaaacaaacaaactaaacaaattaac

SEQ ID NO: 10 - DNA, 1000 bp, Saccharomyces cerevisiae
  source 1..1000: mol_type = genomic DNA
  regulatory 1..1000: regulatory_class = promoter
tccataaacatttcctttgtctatattatccaggggcccaaaataagcccatacccacctatctagagaggctttattgggagtgacgggatatcgtatgttttcctttacctcatcaagaggccggatgaaaagggatgcattggcaaaaattcgataatactcctcattgctgacgacctcatctttgtgataatcgtggcaatactgattaattttgctaaacgttttttcgaatatttttttacttttcttcctactgtcgagaacatccttcgcgcaatgtaaccaagaccctagtgctggttcataagtgcaaagagtagcgtgccgacccttcacatccgtgtcaaatttgtgtgattccaattctttagcacaagcatcaattgctatctggtcccattgcgttcttttcttagttgatgctggttttgctaaagaacctggtgccaaatacaccaacagcagcactaatctagcgaaaagcattcgaaatctgggtgaattaaattaactgcgacaaaattggtgatgtgacataccgaaaacgctacattacacatttgccaagtaaaaaattgacgctccttttttaatgatcgctaatgtaatgaaaagtaaaaatccaattaaacgaggaagcggtaaaatatgattgctgtgtagtaatgccacagtcttcttagtcctacctatgtcctagatttttttttgcaccctgtttgttccgcaccccgctatcccatattttttgatgtatgtgaaggaaagcaatctaagggctcaagagccgagttccgccattctaggaaagggtgcaaaaaaatggggttcaattatcgttcgagatttgttatttttttttttgtaaaagttgatttatggggtatttaagccaagaactatttgaagtattaggaggtaatatgaatgttcgaacagatacaaacaagtacttccactcgcttaaaaataactaataacaataatccctaaa

SEQ ID NO: 11 - DNA, 813 bp, Saccharomyces cerevisiae
  source 1..813: mol_type = genomic DNA
  regulatory 1..813: regulatory_class = promoter
ctttgtgataatcgtggcaatactgattaattttgctaaacgttttttcgaatatttttttacttttcttcctactgtcgagaacatccttcgcgcaatgtaaccaagaccctagtgctggttcataagtgcaaagagtagcgtgccgacccttcacatccgtgtcaaatttgtgtgattccaattctttagcacaagcatcaactgctatctggtcccattgcgttcttttcttagttgatgccggttttgctaaagaacctggtgccaaatataccaacagcagcactaatctagcgaaaagcattcgaaatctgggtgaattaaattaactgcgacaaaattggtgatgtgacatactgaaaacgctacattgcacatttgccaagtaaaaaattgacgctccttttttaatgatcgctaatgtaatgaaaagtaaaaatccaattaaacgaggaagcggtaaaatatgattgctgtgtagtaatgccacagtcttcttagtcctgcctatgtcctagattttttttagcaccctgtttgttccgcaccccgctatcccatattttttgatgtatgtgaaggaaagcaatctaagggctcaagagccgagttccgccattctaggaaagggtgcaaaaaaatggggttcaattatcgttcgagatttgttatttttttttgtaaaagttgatttatggggtatttaagccaagaactatttgaagtattaggaggtaatatgaatgttcgaacagatacaaacaagtacttccactcgcttaaaaataactaataacaataatccctaaa

SEQ ID NO: 12 - DNA, 650 bp, Saccharomyces cerevisiae
  source 1..650: mol_type = genomic DNA
  regulatory 650: regulatory_class = promoter
caaatttgtgtgattccaattctttagcacaagcatcaattgctatctggtcccattgcgttcttttcttagttgatgctggttttgctaaagaacctggtgccaaatacaccaacagcagcactaatctagcgaaaagcattcgaaatctgggtgaattaaattaactgcgacaaaattggtgatgtgacataccgaaaacgctacattacacatttgccaagtaaaaaattgacgctccttttttaatgatcgctaatgtaatgaaaagtaaaaatccaattaaacgaggaagcggtaaaatatgattgctgtgtagtaatgccacagtcttcttagtcctacctatgtcctagatttttttttgcaccctgtttgttccgcaccccgctatcccatattttttgatgtatgtgaaggaaagcaatctaagggctcaagagccgagttccgccattctaggaaagggtgcaaaaaaatggggttcaattatcgttcgagatttgttatttttttttttgtaaaagttgatttatggggtatttaagccaagaactatttgaagtattaggaggtaatatgaatgttcgaacagatacaaacaagtacttccactcgcttaaaaataactaataacaataatccctaaa

SEQ ID NO: 13 - DNA, 453 bp, Saccharomyces cerevisiae
  source 1..453: mol_type = genomic DNA
  regulatory 1..453: regulatory_class = promoter
aaaacgctacattacacatttgccaagtaaaaaattgacgctccttttttaatgatcgctaatgtaatgaaaagtaaaaatccaattaaacgaggaagcggtaaaatatgattgctgtgtagtaatgccacagtcttcttagtcctacctatgtcctagatttttttttgcaccctgtttgttccgcaccccgctatcccatattttttgatgtatgtgaaggaaagcaatctaagggctcaagagccgagttccgccattctaggaaagggtgcaaaaaaatggggttcaattatcgttcgagatttgttatttttttttttgtaaaagttgatttatggggtatttaagccaagaactatttgaagtattaggaggtaatatgaatgttcgaacagatacaaacaagtacttccactcgcttaaaaataactaataacaataatccctaaa

SEQ ID NO: 14 - DNA, 338 bp, Saccharomyces cerevisiae
  source 1..338: mol_type = genomic DNA
  regulatory 1..338: regulatory_class = promoter
tgtgtagtaatgccacagtcttcttagtcctacctatgtcctagatttttttttgcaccctgtttgttccgcaccccgctatcccatattttttgatgtatgtgaaggaaagcaatctaagggctcaagagccgagttccgccattctaggaaagggtgcaaaaaaatggggttcaattatcgttcgagatttgttatttttttttttgtaaaagttgatttatggggtatttaagccaagaactatttgaagtattaggaggtaatatgaatgttcgaacagatacaaacaagtacttccactcgcttaaaaataactaataacaataatccctaaa

SEQ ID NO: 15 - DNA, 254 bp, Saccharomyces cerevisiae
  source 1..254: mol_type = genomic DNA
  regulatory 1..254: regulatory_class = promoter
catattttttgatgtatgtgaaggaaagcaatctaagggctcaagagccgagttccgccattctaggaaagggtgcaaaaaaatggggttcaattatcgttcgagatttgttatttttttttttgtaaaagttgatttatggggtatttaagccaagaactatttgaagtattaggaggtaatatgaatgttcgaacagatacaaacaagtacttccactcgcttaaaaataactaataacaataatccctaaa

SEQ ID NO: 16 - DNA, 1000 bp, Saccharomyces cerevisiae
  source 1..1000: mol_type = genomic DNA
  regulatory 1..1000: regulatory_class = promoter
tggcatcaaatttgggaaaatttagcttactcagacaccaacacattttttcatgagtattgttggaaaatctttccatgcttatcagccggaatacggaacaacggctgctgtgatgccgcaaaggggcaaggaaaaaataagcaacacacaccgaaacaggaggaagaaattccgaacacaagcctaagaagacaccgcagttgttgcaacatgttcacatgttcatccccctgctactacttggaaattaatgtaggggtaattggagtgcgccggcccgtgctttcagaggaacaaaggaagaattgttaaaaaaaggcagtgacaacgagcacgggtgcaccatatggtgtgataagcaacctaggacaaaaagaagtgcgcagttcctgtgtgcggcgccaagacaaatgtttcatatgcttctccaaggggctacgctacctagcctcaccacccgatctctttttttcctgaacgcagaggggaccgtacgaaaaaaaatgttttttaggcaacggagattcgttttatccacgtttaccccacaaaaagtgcaggtacattgtggggccccggcatcgaaaaccagtttttttcctttaaacgctggaaaaaaaggagaaattattggaactttgcagagaatagtccgtaggcaaattgaaaatgttccttaaaaaatttcgtttcttactcattgagattattcagatgccctccgtgccttcattgaaaaaaatccaagagatgtctcggatctgtatgcagattttggcttgcagacaatggagagcaaatgggtatacaatatagaaagcacagaaacatataaaaagagctcgagaaaagacatatggtttgtaactatcttcttcttttttccaatttttctgttttaataataaaaaaacaagaacaaacaagctcaacttgtcttttctaagaacaaagaataaacacaaaaacaaaaagtttttttaattttaatcaaaaa

SEQ ID NO: 17 - DNA, 881 bp, Saccharomyces cerevisiae
  source 1..881: mol_type = genomic DNA
  regulatory 1..881: regulatory_class = promoter
cgcaaaggggcaaggaaaaaataagcaacacacaccgaaacaggaggaagaaattccgaacacaagcctaagaagacaccgcagttgttgcaacatgttcacatgttcatccccctgctactacttggaaattaatgtaggggtaattggagtgcgccggcccgtgctttcagaggaacaaaggaagaattgttaaaaaaaggcagtgacaacgagcacgggtgcaccatatggtgtgataagcaacctaggacaaaaagaagtgcgcagttcctgtgtgcggcgccaagacaaatgtttcatatgcttctccaaggggctacgctacctagcctcaccacccgatctctttttttcctgaacgcagaggggaccgtacgaaaaaaaatgttttttaggcaacggagattcgttttatccacgtttaccccacaaaaagtgcaggtacattgtggggccccggcatcgaaaaccagtttttttcctttaaacgctggaaaaaaaggagaaattattggaactttgcagagaatagtccgtaggcaaattgaaaatgttccttaaaaaatttcgtttcttactcattgagattattcagatgccctccgtgccttcattgaaaaaaatccaagagatgtctcggatctgtatgcagattttggcttgcagacaatggagagcaaatgggtatacaatatagaaagcacagaaacatataaaaagagctcgagaaaagacatatggtttgtaactatcttcttcttttttccaatttttctgttttaataataaaaaaacaagaacaaacaagctcaacttgtcttttctaagaacaaagaataaacacaaaaacaaaaagtttttttaattttaatcaaaaa

SEQ ID NO: 18 - DNA, 532 bp, Saccharomyces cerevisiae
  source 1..532: mol_type = genomic DNA
  regulatory 1..532: regulatory_class = promoter
tttttttcctgaacgcagaggggaccgtacgaaaaaaaatgttttttaggcaacggagattcgttttatccacgtttaccccacaaaaagtgcaggtacattgtggggccccggcatcgaaaaccagtttttttcctttaaacgctggaaaaaaaggagaaattattggaactttgcagagaatagtccgtaggcaaattgaaaatgttccttaaaaaatttcgtttcttactcattgagattattcagatgccctccgtgccttcattgaaaaaaatccaagagatgtctcggatctgtatgcagattttggcttgcagacaatggagagcaaatgggtatacaatatagaaagcacagaaacatataaaaagagctcgagaaaagacatatggtttgtaactatcttcttcttttttccaatttttctgttttaataataaaaaaacaagaacaaacaagctcaacttgtcttttctaagaacaaagaataaacacaaaaacaaaaagtttttttaattttaatcaaaaa

SEQ ID NO: 19 - DNA, 346 bp, Saccharomyces cerevisiae
  source 1..346: mol_type = genomic DNA
  regulatory 1..346: regulatory_class = promoter
tccgtaggcaaattgaaaatgttccttaaaaaatttcgtttcttactcattgagattattcagatgccctccgtgccttcattgaaaaaaatccaagagatgtctcggatctgtatgcagattttggcttgcagacaatggagagcaaatgggtatacaatatagaaagcacagaaacatataaaaagagctcgagaaaagacatatggtttgtaactatcttcttcttttttccaatttttctgttttaataataaaaaaacaagaacaaacaagctcaacttgtcttttctaagaacaaagaataaacacaaaaacaaaaagtttttttaattttaatcaaaaa

SEQ ID NO: 20 - DNA, 210 bp, Saccharomyces cerevisiae
  source 1..210: mol_type = genomic DNA
  regulatory 1..210: regulatory_class = promoter
aatggagagcaaatgggtatacaatatagaaagcacagaaacatataaaaagagctcgagaaaagacatatggtttgtaactatcttcttcttttttccaatttttctgttttaataataaaaaaacaagaacaaacaagctcaacttgtcttttctaagaacaaagaataaacacaaaaacaaaaagtttttttaattttaatcaaaaa

SEQ ID NO: 21 - DNA, 726 bp, Saccharomyces cerevisiae
  source 1..726: mol_type = genomic DNA
  regulatory 1..726: regulatory_class = promoter
acaacacaatggttagtagcaacctgaattcggtcattgatgcatgcatgtgccgtgaagcgggacaaccagaaaagtcgtctataaatgccggcacgtgcgatcatcgtggcggggttttaagagtgcatatcacaaattgtcgcattaccgcggaaccgccagatattcattacttgacgcaaaagcgtttgaaataatgacgaaaaagaaggaagaaaaaaaaagaaaaataccgcttctaggcgggttatctgctaatccgagcttccactaggatagcacccaaacacctgcatatttggacgacctttacttacaccaccaaaaaccactttcgcctctcccgcccctgataacgtccactaattgagcgattacctgagcggtcctcttttgtttgcagcatgagacttgcatactgcaaatcgtaagtagcaacgtctcaaggtcaaaactgtatggaaatcttgtcacctcacctaattctagctagcctaccctgcaagtcaagaggtctccgtgattcctagccacctcaaggtatgcctctccccggaaactgtggccttttctggcacacatgatctccacgatttcaacatataaatagcttttgataatggcaatattaatcaaatttattttacttctttcttgtaacatctctcttgtaattccttattccttctagctatttttcataaaaaaccaagcaactgctta

SEQ ID NO: 22 - DNA, 1716 bp, Bacillus subtilis
  source 1..1716: mol_type = genomic DNA
  CDS 1..1716: gene = ilvB; protein_id = 32; transl_table = 11; translation = MLTKATKEQKSLVKNRGAELVVDCLVEQGVTHVFGIPGAKIDAV FDALQDKGPEIIVARHEQNAAFMAQAVGRLTGKPGVVLVTSGPGASNLATGLLTANTE GDPVVALAGNVIRADRLKRTHQSLDNAALFQPITKYSVEVQDVKNIPEAVTNAFRIAS AGQAGAAFVSFPQDVVNEVTNTKNVRAVAAPKLGPAADDAISAAIAKIQTAKLPVVLV GMKGGRPEAIKAVRKLLKKVQLPFVETYQAAGTLSRDLEDQYFGRIGLFRNQPGDLLL EQADVVLTIGYDPIEYDPKFWNINGDRTIIHLDEIIADIDHAYQPDLELIGDIPSTIN HIEHDAVKVEFAEREQKILSDLKQYMHEGEQVPADWKSDRAHPLEIVKELRNAVDDHV TVTCDIGSHAIWMSRYFRSYEPLTLMISNGMQTLGVALPWAIGASLVKPGEKVVSVSG DGGFLFSAMELETAVRLKAPIVHIVWNDSTYDMVAFQQLKKYNRTSAVDFGNIDIVKY AESFGATGLRVESPDQLADVLRQGMNAEGPVIIDVPVDYSDNINLASDKLPKEFGELM KTKAL
atgttgactaaagctacaaaagagcagaaatcattggtgaaaaataggggtgcagaacttgttgtggactgtttggtagaacagggcgtaacacatgtttttggtatcccaggtgcaaaaatcgacgccgtgtttgatgcattacaagacaagggtccagaaattattgttgctagacatgagcaaaatgccgcatttatggcgcaagctgtaggtaggcttacaggtaaacctggtgttgtcctagttacgtctggcccaggagcctccaatttagcaactggtctattgacagctaatactgagggagatcctgtagttgcgttagccggtaatgtaattagagctgataggcttaagagaactcaccagtctctagacaacgctgctttattccaaccgatcaccaagtactcagtagaggtacaagacgtaaagaatatacctgaagctgtgacaaacgcatttcgtatagcttctgctggtcaggctggtgccgcgtttgtttcttttcctcaagacgttgtcaatgaagtgaccaatactaaaaacgttagagcggttgcagcccctaaactaggtccagccgcagacgacgcaattagcgctgcaattgctaaaattcagacggcgaaactaccagtagtccttgtcggtatgaagggcggaagaccagaagcaataaaagctgttcgtaagttattgaagaaagtccaattacctttcgttgagacttaccaagcagcaggtactttatctagagatttagaggatcagtattttggaaggataggtctatttagaaaccaaccaggagatttactattagaacaagctgatgttgtacttactatcggttatgatcctatagagtatgacccaaagttttggaacataaatggggatagaacaattatacatctagacgagataatcgccgacatcgatcacgcttatcaaccagatttagaactaatcggagatatcccgtcaacaatcaatcatattgaacatgatgctgtaaaggttgagttcgctgaacgtgagcagaaaatcttatctgatctaaagcaatatatgcatgagggtgaacaagttccagcagactggaaatctgaccgtgcacatcctttggaaatcgttaaggaactaagaaatgcggtcgatgatcatgtgactgttacatgtgatatcggttcacatgcaatttggatgtcacgttattttaggagctacgaaccattaactttaatgatatctaacgggatgcaaactctgggggttgcacttccttgggctattggcgctagtttagttaagcccggtgagaaggtggtatcggtatcaggtgatggtggctttctgttttcggctatggaattagaaactgcagtccgtttaaaagctcccattgtgcatattgtctggaatgattctacttacgacatggttgcttttcaacagttgaagaaatacaatagaacttcggctgtagactttggtaacatcgatattgtgaaatatgctgagtcttttggcgcaacaggcctgagggtggaaagtccagatcagttagctgatgtgttgagacaagggatgaatgccgagggaccggtaatcatagatgtgccagttgactactcagacaatattaatttggcttctgataaacttcctaaagagtttggcgagctaatgaagaccaaagccttataa

SEQ ID NO: 23 - DNA, 1035 bp, Slackia exigua
  source 1..1035: mol_type = genomic DNA
  CDS 1..1035: gene = ilvC; protein_id = 33; transl_table = 11; translation = MSVKTKEKEMAVTILYEQDVDPKVIQGLKVGIIGYGSQGHAHAL NLMDSGVDVRVGLREGDSDWKTAEEAGLKVTDIDTAAEEADVIMVLIPDEVQPKVYQE HIAAHLKAGNTLAFAHGFNIHYGYIVPPEDVNVIMCAPKGPGHIVRRQFTEGSGVPDL ACVQQDATGNAWDIVLSYCWGVGGARSGIIKATFAEETEEDLFGEQAVLCGGLVELVK AGFETLTEAGYPPELAYFECYHEMKMIVDLMYESGIHFMNYSISNTAEYGEYYAGPKV INEQSREAMKEILARIQDGSFAQEFVDDCNNGHKRLLEQREAINTHPIETTGARIRSM FSWIKKEDLE
atgtccgtcaagacaaaagaaaaggaaatggcagttacaattttatacgaacaagacgttgatccaaaggttatccaaggtttgaaggttggtatcataggttacggttctcaaggtcacgctcatgcattgaacttaatggattcaggtgttgatgtcagagttggtttgagagaaggtgactctgattggaagacagccgaagaagctggtttaaaggttacagatattgacactgccgcagaagaagcagatgttattatggtcttaattcctgatgaagttcaaccaaaggtttaccaagaacatattgcagctcatttgaaggctggtaataccttggcttttgcacacggtttcaatatccattacggttatatcgttcctccagaagacgttaatgtcatcatgtgtgctcctaaaggtccaggtcacatcgttagaagacaattcaccgaaggttcaggtgtccctgatttggcatgtgttcaacaagatgcaactggtaatgcatgggatattgttttatcctattgttggggtgttggtggtgcaagatcaggtatcataaaagctacattcgcagaagaaacagaagaagacttgtttggtgaacaagcagttttgtgtggtggtttagttgaattggtcaaagctggttttgaaaccttgactgaagctggttatccacctgaattggcctactttgaatgttatcacgaaatgaagatgatcgttgacttgatgtatgaatctggtattcattttatgaactactcaatctctaacactgcagaatacggtgaatactatgccggtcctaaagtcatcaatgaacaaagtagagaagcaatgaaagaaatcttggctagaatccaagacggttcatttgctcaagaatttgtcgatgactgcaataatggtcataagagattgttagaacaaagagaagctattaacactcatccaatcgaaactacaggtgctagaatcagatcaatgttttcttggataaagaaggaagatttggaatga

SEQ ID NO: 24 - DNA, 1713 bp, Lactococcus lactis
  source 1..1713: mol_type = genomic DNA
  CDS 1..1713: gene = ilvD; protein_id = 34; transl_table = 11; translation = MEFKYNGKTEAIELNKYSKTLTQDPTQPATQAMYYGIGFKDEDF KKAQVGIVSMDWDGNPCNMHLGTLGGKIKASVNQTEGLIGLQFHTIGVSDGIANGKLG MRYSLVSREVIADSIETNAGAEYYDAIVSVPGCDKNMPGSIIGMARLNRPSIMVYGGT IEHGEYKGEKLNIVSAFEALGQKITGNISEEDYHGCIKNAIPGAGACGGMYTANTLAS AIETLGMSLPYSASNPAVSPEKQEECDEIGAAIKNLLEKDIKPSDIMTKEAFENAITI VMVLGGSTNAVLHIIAMANAIGVEITQDDFQRISDVTPVLGDFKPSGKYMMEDLHKIG GVPAVLKYLLKEGKLHGDCLTVTGKTLAENVANALDLDFENQDIMHSLSDPIKATGHL QILYGNLAEGGSVAKISGKEGEFFKGTARVFDGEQHFIDGIESGRLHAGDVAVIRNIG PVGGPGMPEMLKPTSALIGAGLGKSCALITDGRFSGGTHGFVVGHIVPEAVEGGLIGL VEDEDIIEIDATNNTITLKVDDEEIARRRAAYVRPLPKATRGVLAKFAKLTRPASEGC VTDL
atggaatttaaatataatggtaaaacagaagccatcgaacttaacaaatactcaaaaacactaacccaagatcccactcaaccagcaactcaagcgatgtactacggtatcggttttaaagatgaagactttaaaaaagcccaagtcggcattgtcagtatggactgggacggaaatccctgtaatatgcatcttggaacacttggtgggaaaatcaaagcctctgtcaatcaaacagaaggattaattggtttacaatttcatacgatcggcgtatcggatgggattgcaaacggtaaacttggaatgagatatagcttggtaagccgtgaggtcattgctgacagtattgagaccaacgctggcgcagaatactacgatgcgattgtttctgtaccaggctgtgacaaaaatatgccaggctcgattattggaatggcgagactcaatcgaccttcaatcatggtttacggtggaacaatagaacacggtgaatacaaaggtgaaaaactaaacattgtttcagcttttgaagcattagggcaaaaaattacaggtaatatctccgaagaagattatcatggttgtatcaaaaatgcaattccaggagctggggcttgtggtggaatgtatacagcaaatacattagcgtcagcaattgaaacgcttggaatgagtttgccatattctgcatcaaatcctgcggtcagtccagaaaaacaagaagaatgtgatgaaatcggcgcagcaatcaaaaatttgcttgaaaaggatattaagccaagtgatattatgaccaaagaagcgtttgaaaacgcaattacgattgtcatggtgcttggtggctcgactaatgcggtattacatatcattgcaatggcaaatgccatcggtgttgaaattacgcaagacgacttccaaagaatttcagatgtaacacctgttcttggtgattttaagccaagcggaaagtacatgatggaagacttacacaaaattggtggtgtgcctgccgttctcaaatatttgctcaaagaaggtaaacttcacggagactgtttaacggttacaggtaaaactttagctgaaaatgttgccaatgcgcttgatttggactttgaaaatcaagacatcatgcattcgctttctgatccaatcaaagcaacaggacatttacaaattttatatggcaacctcgctgaaggtggttctgtcgcaaaaatttctggtaaagaaggcgaatttttcaaaggaacggcgcgagtctttgatggcgagcaacatttcattgatgggattgagtctggaaggttgcacgcaggcgatgttgctgtcatacgtaatattggtcctgtcggtggtccaggaatgcctgaaatgcttaaaccaacctctgctttgattggtgctggacttggcaaatcttgtgcgctgattacagatggaagattttcaggtgggacacacggatttgttgtaggtcatattgttcctgaagctgttgaaggtggattgatcggtttagttgaagatgaggacatcatcgaaattgatgctactaacaatacgataacattgaaagttgatgatgaggaaatcgcgcgtcgtcgtgcagcatacgtgcgtccgttacccaaagcaacacgtggtgtactcgcaaaatttgcaaaactgacacgacctgctagtgagggctgtgtgacagatttatga

SEQ ID NO: 25 - DNA, 1647 bp, Lactococcus lactis
  source 1..1647: mol_type = genomic DNA
  CDS 1..1647: gene = ilvE; protein_id = 35; transl_table = 11; translation = MYTVGDYLLDRLHELGIEEIFGVPGDYNLQFLDQIISRKDMKWV GNANELNASYMADGYARTKKAAAFLTTFGVGELSAVNGLAGSYAENLPVVEIVGSPTS KVQNEGKFVHHTLADGDFKHFMKMHEPVTAARTLLTAENATVEIDRVLSALLKERKPV YINLPVDVAAAKAEKPSLPLKKENSTSNTSDQEILNKIQESLKNAKKPIVITGHEIIS FGLEKTVSQFISKTKLPITTLNFGKSSVDEALPSFLGIYNGKLSEPNLKEFVESADFI LMLGVKLTDSSTGAFTHHLNENKMISLNIDEGKIFNESIQNFDFESLISSLLDLSEIE YKGKYIDKKQEDFVPSNALLSQDRLWQAVENLTQSNETIVAEQGTSFFGASSIFLKPK SHFIGQPLWGSIGYTFPAALGSQIADKESRHLLFIGDGSLQLTVQELGLAIREKINPI CFIINNDGYTVEREIHGPNQSYNDIPMWNYSKLPESFGATEERVVSKIVRTENEFVSV MKEAQADPNRMYWIELILAKEDAPKVLKKMGKLFAEQNKS
atgtatacagtgggtgattacttgctagaccgtttacatgaattaggcatagaagagatttttggagtaccaggtgattacaatttgcaattcttggatcagattatctcaagaaaggatatgaaatgggtcgggaatgcgaacgagttaaacgcttcctatatggcagatggttatgcgagaaccaaaaaggccgctgcttttcttacaactttcggtgtaggtgaactttcagccgttaatggattagccggatcttacgctgaaaacttaccagtcgttgaaattgttggttctcctacttctaaggtacaaaacgagggaaagtttgttcaccatactttagcggatggtgatttcaaacatttcatgaagatgcatgaacctgtcacagcagcgagaacacttttgaccgcagagaacgctactgttgaaatcgatagagtattaagtgctttgttaaaagagagaaagccagtgtatatcaacttgcctgttgatgtcgctgccgcaaaagcagaaaaaccatctttaccattgaaaaaggaaaactcaaccagtaacacatctgatcaagagattctaaacaaaatccaagagtcattgaaaaacgccaaaaagccaatcgttataacaggccatgaaatcatctcctttggtttagaaaagaccgtttcacaattcatctctaagacaaaattgccaatcactactttaaactttggcaaatcttctgtagatgaagctttaccatcttttctaggtatctacaatggtaaactttctgaaccaaacctaaaagagtttgtcgaatccgctgatttcatacttatgttgggtgttaagctaactgatagttcaactggtgcattcactcaccacttgaacgaaaacaagatgatatcattgaatatcgatgagggcaagatattcaacgaatcaattcaaaacttcgattttgaatcactaatttcttctctacttgatttgtcagaaatagaatacaaaggaaaatacatcgacaaaaagcaagaggatttcgtgccatcaaacgcattgttgtcacaagatagactttggcaagctgtggaaaacctaactcaatctaacgaaacaattgtggctgagcaaggcacatcattcttcggcgcatctagtattttcttgaagccaaagtcccacttcatcggtcaacctctttggggatctattgggtacaccttccctgccgcattgggatcacagatcgcagacaaggagtccagacatctactattcattggggacggatcattgcaacttaccgttcaggaattagggttggctataagagaaaagatcaatcctatttgttttatcatcaacaatgatggctatacagtagaacgtgagattcacggtccaaatcaaagttacaatgacatcccaatgtggaattactctaagttgcctgaatctttcggtgcaacagaagaaagagttgtttccaaaatagtcagaacagaaaatgagtttgtttctgtcatgaaggaagctcaagccgatcctaatagaatgtactggattgaactaatcttggccaaggaagatgccccaaaagtattgaaaaagatgggaaaacttttcgctgaacagaataagtcctga

SEQ ID NO: 26 - DNA, 1023 bp, Lactococcus lactis
  source 1..1023: mol_type = genomic DNA
  CDS 1..1023: gene = ADH1; protein_id = 36; transl_table = 11; translation = MKAAVVRHNPDGYADLVEKELRAIKPNEALLDMEYCGVCHTDLH VAAGDFGNKAGTVLGHEGIGIVKEIGADVSSLQVGDRVSVAWFFEGCGHCEYCVSGNE TFCREVKSAGYSVDGGMAEEAIVVADYAVKVPDGLDPIEASSITCAGVTTYKAIKVSG VKPGDWQVIFGAGGLGNLAIQYAKNVFGAKVIAVDINQDKLNLAKKIGADVTINSGDV NPVDEIKKITGGLGVQSAIVCAVARIAFEQAVASLKPMGKMVAVAVPNTEMTLSVPTV VFDGVEVAGSLVGTRLDLAEAFQFGAEGKVKPIVATRKLEEINDIIDEMKAGKIEGRM VIDFTK
atgaaagcagcagtagtaagacacaatccagatggttatgcggaccttgttgaaaaggaacttcgagcaatcaaacctaatgaagctttgcttgacatggagtattgtggagtctgtcataccgatttgcacgtagcagcaggtgattttggcaacaaagcagggactgttcttggtcatgaaggaattggaattgtcaaagaaattggagctgatgtaagctcgcttcaagttggtgatcgggtttcagtggcttggttctttgaaggatgtggtcactgtgaatactgtgtatctggtaatgaaactttttgtcgagaagttaaaagtgcaggatattcagttgatggcggaatggctgaagaagcaattgttgttgccgattatgctgtcaaagttcctgacggacttgacccaattgaagctagctcaattacttgtgctggagtaacaacttacaaagcaatcaaagtatcaggagtaaaacctggtgattggcaagtaatttttggtgctggaggacttggaaatttagcaattcaatatgctaaaaatgtttttggagcaaaagtaattgctgttgatattaatcaagataaattaaatttagctaaaaaaattggagctgatgtgactatcaattctggtgatgtaaatccagttgatgaaattaaaaaaataactggcggcttaggggtgcaaagtgcaatagtttgtgctgttgcaaggattgcttttgaacaagcggttgcttctttgaaacctatgggcaaaatggttgctgtggcagttcccaatactgagatgactttatcagttccaacagttgtttttgacggagtggaggttgcaggttcacttgtaggaacaagacttgacttggcagaagcttttcaatttggagcagaaggtaaggtaaaaccaattgttgcgacacgcaaactggaagaaatcaatgatattattgatgaaatgaaggcaggaaaaattgaaggccgaatggtcattgattttactaaataa

SEQ ID NO: 27 - protein, 571 aa, Bacillus subtilis
MLTKATKEQKSLVKNRGAELVVDCLVEQGVTHVFGIPGAKIDAVFDALQDKGPEIIVARHEQNAAFMAQAVGRLTGKPGVVLVTSGPGASNLATGLLTANTEGDPVVALAGNVIRADRLKRTHQSLDNAALFQPITKYSVEVQDVKNIPEAVTNAFRIASAGQAGAAFVSFPQDVVNEVTNTKNVRAVAAPKLGPAADDAISAAIAKIQTAKLPVVLVGMKGGRPEAIKAVRKLLKKVQLPFVETYQAAGTLSRDLEDQYFGRIGLFRNQPGDLLLEQADVVLTIGYDPIEYDPKFWNINGDRTIIHLDEIIADIDHAYQPDLELIGDIPSTINHIEHDAVKVEFAEREQKILSDLKQYMHEGEQVPADWKSDRAHPLEIVKELRNAVDDHVTVTCDIGSHAIWMSRYFRSYEPLTLMISNGMQTLGVALPWAIGASLVKPGEKVVSVSGDGGFLFSAMELETAVRLKAPIVHIVWNDSTYDMVAFQQLKKYNRTSAVDFGNIDIVKYAESFGATGLRVESPDQLADVLRQGMNAEGPVIIDVPVDYSDNINLASDKLPKEFGELMKTKAL

SEQ ID NO: 28 - protein, 344 aa, Slackia exigua
MSVKTKEKEMAVTILYEQDVDPKVIQGLKVGIIGYGSQGHAHALNLMDSGVDVRVGLREGDSDWKTAEEAGLKVTDIDTAAEEADVIMVLIPDEVQPKVYQEHIAAHLKAGNTLAFAHGFNIHYGYIVPPEDVNVIMCAPKGPGHIVRRQFTEGSGVPDLACVQQDATGNAWDIVLSYCWGVGGARSGIIKATFAEETEEDLFGEQAVLCGGLVELVKAGFETLTEAGYPPELAYFECYHEMKMIVDLMYESGIHFMNYSISNTAEYGEYYAGPKVINEQSREAMKEILARIQDGSFAQEFVDDCNNGHKRLLEQREAINTHPIETTGARIRSMFSWIKKEDLE

SEQ ID NO: 29 - protein, 570 aa, Lactococcus lactis
MEFKYNGKTEAIELNKYSKTLTQDPTQPATQAMYYGIGFKDEDFKKAQVGIVSMDWDGNPCNMHLGTLGGKIKASVNQTEGLIGLQFHTIGVSDGIANGKLGMRYSLVSREVIADSIETNAGAEYYDAIVSVPGCDKNMPGSIIGMARLNRPSIMVYGGTIEHGEYKGEKLNIVSAFEALGQKITGNISEEDYHGCIKNAIPGAGACGGMYTANTLASAIETLGMSLPYSASNPAVSPEKQEECDEIGAAIKNLLEKDIKPSDIMTKEAFENAITIVMVLGGSTNAVLHIIAMANAIGVEITQDDFQRISDVTPVLGDFKPSGKYMMEDLHKIGGVPAVLKYLLKEGKLHGDCLTVTGKTLAENVANALDLDFENQDIMHSLSDPIKATGHLQILYGNLAEGGSVAKISGKEGEFFKGTARVFDGEQHFIDGIESGRLHAGDVAVIRNIGPVGGPGMPEMLKPTSALIGAGLGKSCALITDGRFSGGTHGFVVGHIVPEAVEGGLIGLVEDEDIIEIDATNNTITLKVDDEEIARRRAAYVRPLPKATRGVLAKFAKLTRPASEGCVTDL

SEQ ID NO: 30 - protein, 548 aa, Lactococcus lactis
MYTVGDYLLDRLHELGIEEIFGVPGDYNLQFLDQIISRKDMKWVGNANELNASYMADGYARTKKAAAFLTTFGVGELSAVNGLAGSYAENLPVVEIVGSPTSKVQNEGKFVHHTLADGDFKHFMKMHEPVTAARTLLTAENATVEIDRVLSALLKERKPVYINLPVDVAAAKAEKPSLPLKKENSTSNTSDQEILNKIQESLKNAKKPIVITGHEIISFGLEKTVSQFISKTKLPITTLNFGKSSVDEALPSFLGIYNGKLSEPNLKEFVESADFILMLGVKLTDSSTGAFTHHLNENKMISLNIDEGKIFNESIQNFDFESLISSLLDLSEIEYKGKYIDKKQEDFVPSNALLSQDRLWQAVENLTQSNETIVAEQGTSFFGASSIFLKPKSHFIGQPLWGSIGYTFPAALGSQIADKESRHLLFIGDGSLQLTVQELGLAIREKINPICFIINNDGYTVEREIHGPNQSYNDIPMWNYSKLPESFGATEERVVSKIVRTENEFVSVMKEAQADPNRMYWIELILAKEDAPKVLKKMGKLFAEQNKS

SEQ ID NO: 31 - protein, 340 aa, Lactococcus lactis
MKAAVVRHNPDGYADLVEKELRAIKPNEALLDMEYCGVCHTDLHVAAGDFGNKAGTVLGHEGIGIVKEIGADVSSLQVGDRVSVAWFFEGCGHCEYCVSGNETFCREVKSAGYSVDGGMAEEAIVVADYAVKVPDGLDPIEASSITCAGVTTYKAIKVSGVKPGDWQVIFGAGGLGNLAIQYAKNVFGAKVIAVDINQDKLNLAKKIGADVTINSGDVNPVDEIKKITGGLGVQSAIVCAVARIAFEQAVASLKPMGKMVAVAVPNTEMTLSVPTVVFDGVEVAGSLVGTRLDLAEAFQFGAEGKVKPIVATRKLEEINDIIDEMKAGKIEGRMVIDFTK

SEQ ID NO: 32 - protein, 571 aa, Bacillus subtilis; identical to SEQ ID NO: 27

SEQ ID NO: 33 - protein, 344 aa, Slackia exigua; identical to SEQ ID NO: 28

SEQ ID NO: 34 - protein, 570 aa, Lactococcus lactis; identical to SEQ ID NO: 29

SEQ ID NO: 35 - protein, 548 aa, Lactococcus lactis; identical to SEQ ID NO: 30

SEQ ID NO: 36 - protein, 340 aa, Lactococcus lactis; identical to SEQ ID NO: 31